(19) United States
(12) Patent Application Publication
(10) Pub. No.: US 2006/0111355 A1
(43) Pub. Date: May 25, 2006

(54) Gonadotropin releasing hormone receptor antagonists

(75) Inventors: Lloyd Michael Garrick, Arvada, CO (US); Daniel Michael Green, Ambler, PA (US); James Winfield Jetter, Norristown, PA (US); Wenling Kao, Fremont, CA (US); Kenneth Lewis Kees, Glenmoore, PA (US); Jeffrey Claude Pelletier, Lafayette Hill, PA (US); John Francis Rogers JR., Bryn Mawr, PA (US)

Correspondence Address: WILMER CUTLER PICKERING HALE AND DORR LLP / WYETH, 60 STATE STREET, BOSTON, MA, 02109, US

(73) Assignee: WYETH, Madison, NJ (US)

(21) Appl. No.: 11/286,081

(22) Filed: Nov. 23, 2005

Related U.S. Application Data
(60) Provisional application No. 60/630,282, filed on Nov. 23, 2004.

(51) Int. Cl. A61K 31/506; A61K 31/496; A61K 31/501; A61K 31/498; C07D 403/14; C07D 413/14; C07D 417/14

(52) U.S. Cl. 514/248; 514/254.09; 514/249; 514/252.19; 514/252.11; 514/253.06; 514/254.05; 514/254.06; 514/253.05; 544/236; 544/295; 544/333; 544/357; 544/360; 544/363; 544/368; 544/373; 514/254.02

(57) ABSTRACT

The present invention relates to Gonadotropin Releasing Hormone (“GnRH”) (also known as Luteinizing Hormone Releasing Hormone) receptor antagonists.

This application claims the benefit of provisional application U.S. Ser. No. 60/630,282, filed Nov. 23, 2004, which is hereby incorporated by reference into the subject application in its entirety.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF INVENTION

The present invention relates to Gonadotropin Releasing Hormone (“GnRH”) (also known as Luteinizing Hormone Releasing Hormone) receptor antagonists, processes for preparing them and to pharmaceutical compositions containing them.

### BACKGROUND

GnRH is a decameric peptide released from the hypothalamus. In the anterior pituitary gland, GnRH activates the GnRH receptor. Activation of the GnRH receptor triggers the release of follicle stimulating hormone (FSH) and luteinizing hormone (LH). FSH and LH stimulate the biosynthesis and release of sex steroids in the gonads of both genders.

Typically, this is desirable, but certain sex hormone dependent pathological conditions exist where it would be beneficial to prevent activation of the GnRH receptor. For example, inhibition of the GnRH receptor can lead to a large drop in sex steroid production, which in turn can alleviate sex hormone dependent pathological conditions such as prostate cancer, endometriosis, uterine fibroids, uterine cancer, breast cancer, ovarian cancer, testicular cancer, or primary hirsutism. Moreover, there are other situations where it would be beneficial to prevent activation of the GnRH receptor, such as during some points of the in vitro fertilization process, such as to prevent LH surge.

All currently marketed GnRH therapeutics are peptides that exhibit receptor antagonism in one of two ways. The first is through GnRH receptor superagonism. The GnRH receptor, when stimulated in bursts, causes normal release of the gonadotropins, FSH and LH. Under constant stimulation, the receptor becomes desensitized and the overall effect is GnRH receptor inhibition. The superagonism process is somewhat undesirable, as inhibition via this process can take up to two weeks to arise in human patients. During this delay there is often an increase in disease symptoms due to the initial hormone stimulation phase. This phenomenon is referred to as flare.

The second method for receptor inhibition is through direct antagonism of the GnRH receptor with peptide antagonists. This causes an immediate drop in plasma LH levels. However, as mentioned above, current pharmaceuticals that cause blockade of the GnRH receptor are all peptides. As such they are not orally bioavailable and must be administered via parenteral means such as intravenous, subcutaneous or intramuscular injection. Thus, an orally effective GnRH antagonist would be of significant benefit.

Therefore, based upon the foregoing, it is clear that GnRH receptor antagonists are useful, and development of new GnRH receptor antagonists is highly desirable.

### SUMMARY

The present invention relates to compounds, and methods of use for compounds, of the formula I: 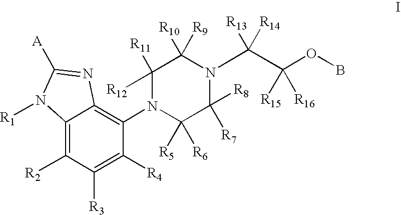
or a pharmaceutically acceptable salt thereof, wherein:

A is cycloalkyl, aryl, heteroaryl, or diaryl substituted alkyl, each optionally substituted;

B is aryl or heteroaryl, each optionally substituted;

R₁ is H, the tautomeric form, or optionally substituted alkyl;

R₂, R₃, and R₄ are, independently, H, optionally substituted alkyl, halogen, or OR₁; and

R₅, R₆, R₇, R₈, R₉, R₁₀, R₁₁, R₁₂, R₁₃, R₁₄, R₁₅, and R₁₆, are, independently, H, alkyl, alkenyl, or alkynyl, each alkyl, alkenyl, or alkynyl being optionally substituted.

### DETAILED DESCRIPTION

The present invention relates to compounds, and methods of use for compounds, of Formula I: 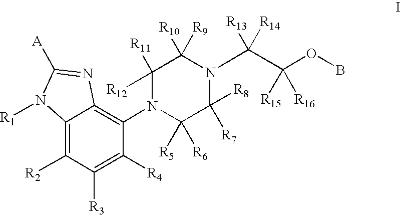
or a pharmaceutically acceptable salt thereof, wherein:

A is cycloalkyl, aryl, heteroaryl, or diaryl substituted alkyl, each optionally substituted;

B is aryl or heteroaryl, each optionally substituted;

R₁ is H, the tautomeric form, or optionally substituted alkyl;

R₂, R₃, and R₄ are, independently, H, optionally substituted alkyl, halogen, or OR₁; and

R₅, R₆, R₇, R₈, R₉, R₁₀, R₁₁, R₁₂, R₁₃, R₁₄, R₁₅, and R₁₆, are, independently, H, alkyl, alkenyl, or alkynyl, each alkyl, alkenyl, or alkynyl being optionally substituted.

For clarity of presentation, the use of “optionally substituted” has, in some instances, been avoided. However, it is understood that unless stated otherwise, each alkyl, alkenyl, alkynyl, cycloalkyl, cycloheteroalkyl, aryl, or heteroaryl is contemplated as being optionally substituted. This paragraph is intended to make clear that when the description and claims refer to a moiety, it encompasses both substituted and unsubstituted forms of said moiety.

In some embodiments, B is: 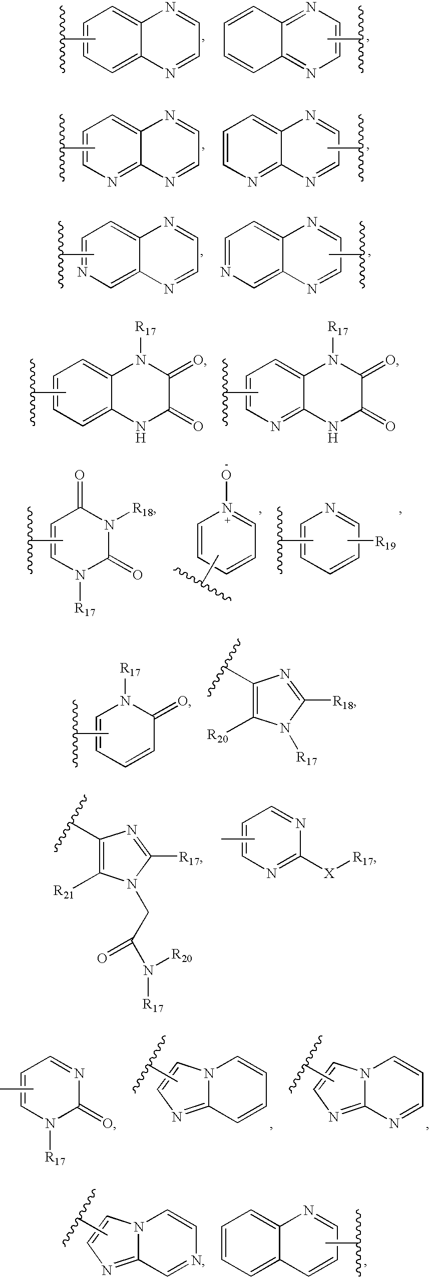 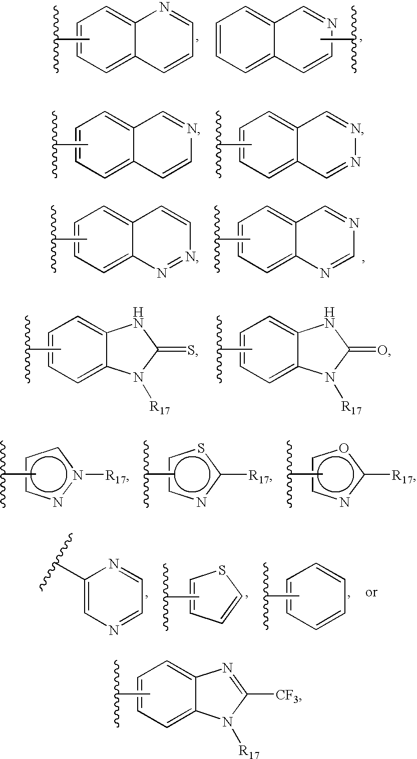
each B also having up to three R₂₀ substituents attached to the ring of B containing at least one N; wherein:

R₁₇ is hydrogen, alkyl, alkenyl, alkynyl, aryl, heteroaryl, arylalkyl, heteroarylalkyl, R₂₂XR₂₃, COXR₂₂, or XR₂₂, wherein X is O, NR₂₃, S, SO, or SO₂;

R₁₈ is hydrogen, alkyl, alkenyl, alkynyl, CO₂R₂₂, or CONR₂₂R₂₃;

R₁₉ is hydrogen, CO₂R₂₂, CONR₂₂R₂₃, S, SR₂₂, SO₂, SO₂R₂₂, or SO₃;

R₂₀ and R₂₁ are, independently, H, alkyl, alkenyl, or alkynyl; and

R₂₂ and R₂₃ are, independently, H or alkyl, alternatively R₂₂ and R₂₃, taken together with the atoms to which they are attached, form a 3-7 membered heterocycle, having 1-3 heteroatoms selected from N, O, and S.

In one embodiment, B is of Formula II: 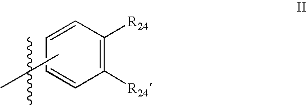
wherein:

R₂₄ and R₂₄′ are, independently, H, optionally substituted alkyl, halogen, NO₂, NHR₂₅, CONHR₂₅, OCONHR₂₅, NHCON(R₂₅)₂, NHCONHCOR₂₅, NHCOR₂₅, NHCO₂R₂₅, NHSO₂R₂₅, OH;

alternatively R₂₄ and R₂₄′, taken together with the atoms to which they are attached, form an optionally substituted 3-7 membered heterocycle, having 1-3 heteroatoms selected from N, O, and S; and

R₂₅ is, independently, H, CF₃, O-alkyl, alkyl, alkenyl, cycloalkyl, heterocycloalkyl, aryl, heteroaryl, arylalkyl, heteroarylalkyl, or CHNHCONH-alkyl, each optionally substituted. In some embodiments, the 3-7 membered heterocycle includes pyrrolidine, piperidine, hexamethyleneimine, piperazine, homopiperazine, aziridine, and azetidine.

In one embodiment of Formula II, R₂₄ and R₂₄′ are independently, NHR₂₅, CONHR₂₅, OCONHR₂₅, NHCONHR₂₅, NHCONHCOR₂₅, NHCOR₂₅, NHCO₂R₂₅, NHSO₂R₂₅; and

R₂₅ is aryl or heterocycloalkyl, optionally substituted with one or more, e.g., 1, 2 or 3 the same or different, of alkyl, halogen, CF₃, O-alkyl, S-alkyl, CO₂alkyl, COalkyl, COH, NO₂ or OH.

In one embodiment of Formula II, R₂₄ and R₂₄′ are independently, NHR₂₅, CONHR₂₅, OCONHR₂₅, NHCONHR₂₅, NHCONHCOR₂₅, NHCOR₂₅, NHCO₂R₂₅, NHSO₂R₂₅; and

R₂₅ is alkyl, optionally substituted with one or more, e.g., 1, 2 or 3 the same or different, of halogen, CF₃, cycloalkyl or OH.

In a further embodiment of Formula II, B is of Formula III: 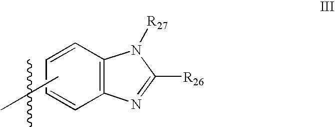
or a tautomeric form thereof, wherein:

R₂₆ is alkyl, S, SR₂₇, CF₃, NH, or NHR₂₇;

R₂₇ is, independently, H, alkyl, CN, CO₂R₂₈, or C(═O)R₂₈; and

R₂₈ is alkyl.

In some embodiments, A or B is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃₁ and R₃₂, taken together with the atoms to which they are attached, form a 3-7 membered heterocycle, having 1-3 heteroatoms selected from N, O, and S.

Substituents on B may themselves be substituted, for example, referring to Formula II, in some embodiments R₂₄ or R₂₄ is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃, and R₃₂ are, independently, H or alkyl, alternatively R₂₉ and R₃₀ or R₃₁ and R₃₂, taken together with the atoms to which they are attached, form a 3-7 membered heterocycle, having 1-3 heteroatoms selected from N, O, and S.

Likewise, referring to Formula III, in some embodiments R₂₆ or R₂₇ is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃, and R₃₂, taken together with the atoms to which they are attached, form a 3-7 membered heterocycle, having 1-3 heteroatoms selected from N, O, and S.

In some embodiments of the present invention, A is phenyl, naphthyl, thiophenyl, or pyridyl. In some embodiments, A is phenyl, 2-thiophenyl, 3-thiophenyl, 2-pyridyl, 3-pyridyl, or 4-pyridyl. It is understood that reference to these A moieties includes substitutions as described above. For example, in some embodiments, A is substituted with at least one, e.g. 1, 2 or 3 the same or different of alkyl, alkenyl, alkynyl, alkoxy, cycloalkyl, heteroalkyl, heterocycloalkyl, aryl, arylalkyl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃, and R₃₂, taken together with the atoms to which they are attached, form a 3-7 membered heterocycle, having 1-3 heteroatoms selected from N, O, and S. Any substituent group on A may be further substituted.

In one embodiment, A is phenyl, 2-pyridyl, 3-pyridyl, 4-pyridyl, 2-naphthyl, 3-naphthyl, 2-thiophenyl, 3-thiophenyl, cyclohexyl, 2,2-diphenylethyl, diphenylmethyl or 2-benzothiophenyl, each optionally substituted.

In another embodiment, A is optionally substituted with one or more of —CN, —OCH₃, —OCH₂CH₃, —O(CH₂)₂CH₃, —O(CH₂)₃CH₃, —O(CH₂)₄CH₃, —O(CH₂)₅CH₃, —O(CH₂)₆CH₃, —F, —Br, —Cl, —I, methyl, ethyl, n-propyl, isopropyl, n-butyl, isobutyl, t-butyl, pentyl, hexyl, heptyl, —CF₃, —OH, —OCF₃, —SCF₃, —NH₂, —NHCH₃, —NHCH₂CH₃, —NH(CH₂)₂CH₃, —NH(CH₂)₃CH₃, —NH(CH₂)₄CH₃, —N(CH₃)₂, —N(CH₂CH₃)₂, —N[(CH₂)₂CH₃]₂, —N[(CH₂)₃CH₃]₂, —N[(CH₂)₃CH₃]₂, —N[(CH₂)₅CH₃]₂, —O-phenyl-OH, —NHC(O)—CH₃, pyrrole, —NO₂, —SH, —SCH₃, —SCH₂CH₃, —CH═CH₂, —C(O)-phenyl, —SO₂CH₃, —SO₂NH₂, benzyl, benzyl substituted with —OH, or —C(O)NH₂.

In one embodiment, B is benzimidazole or phenyl, each optionally substituted.

In one embodiment, B is

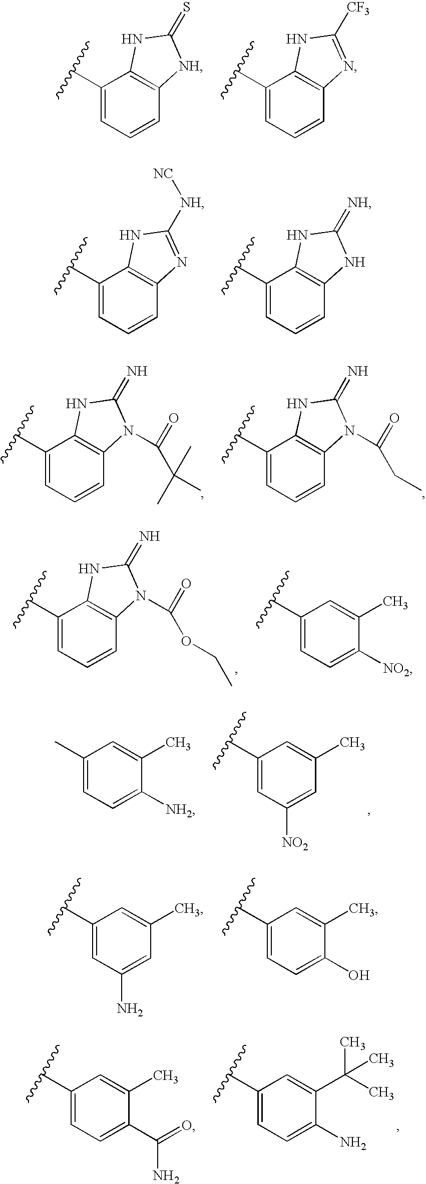

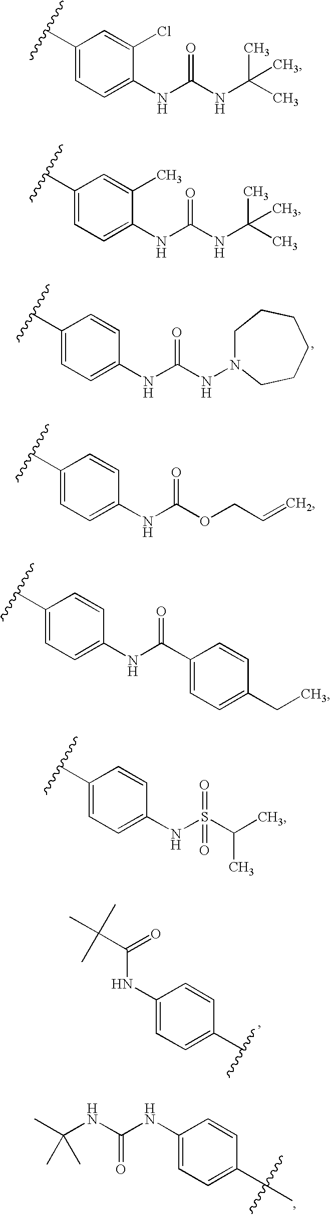

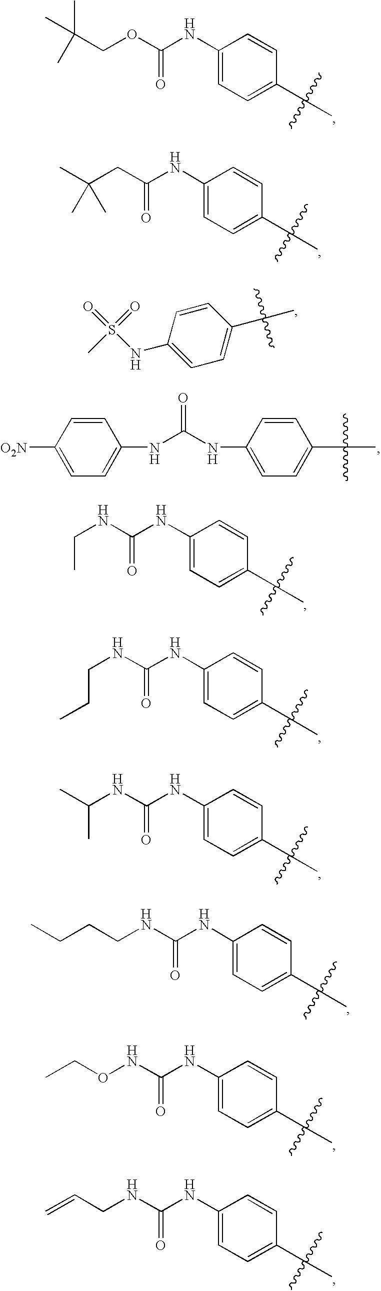

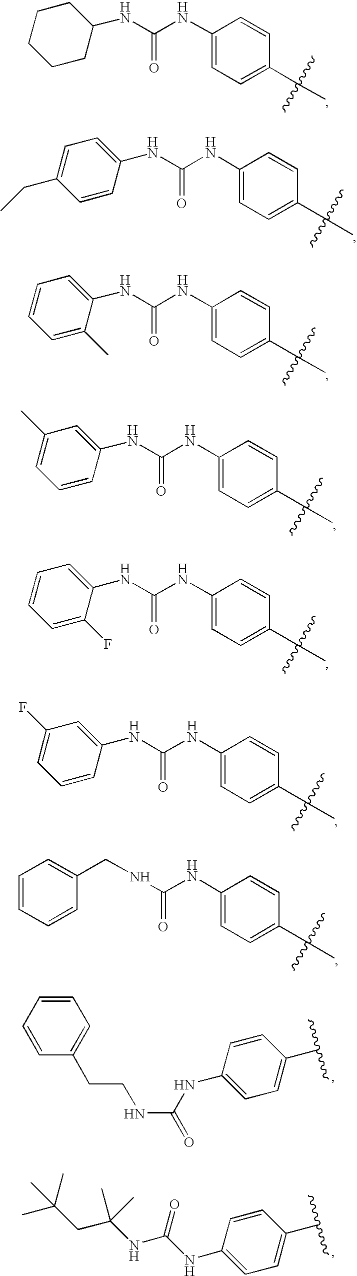

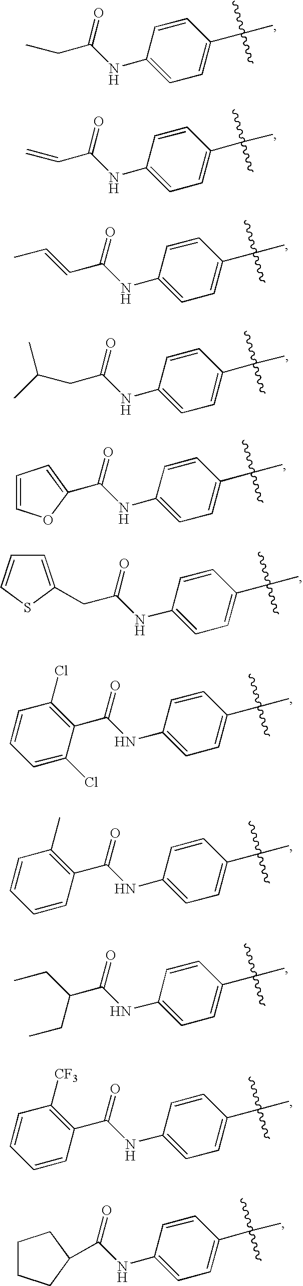

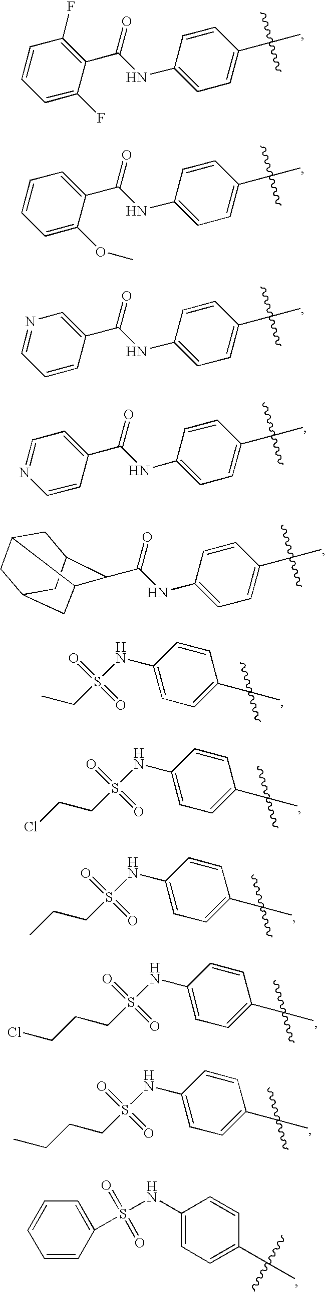

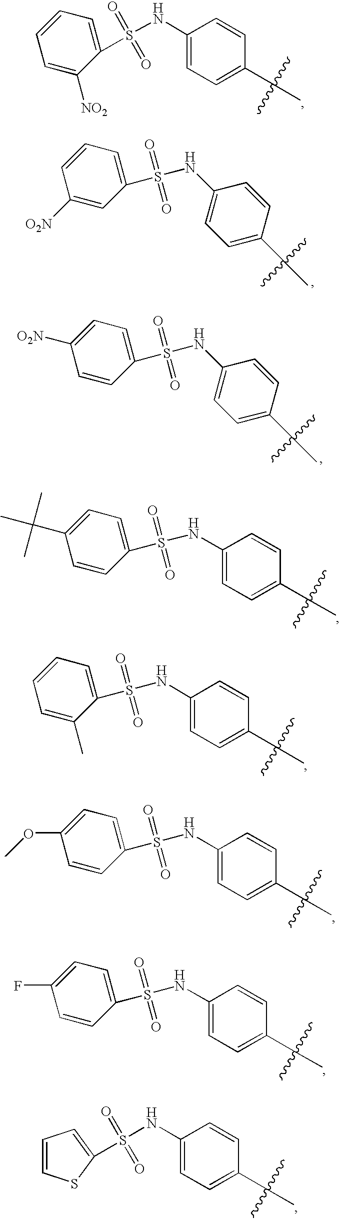

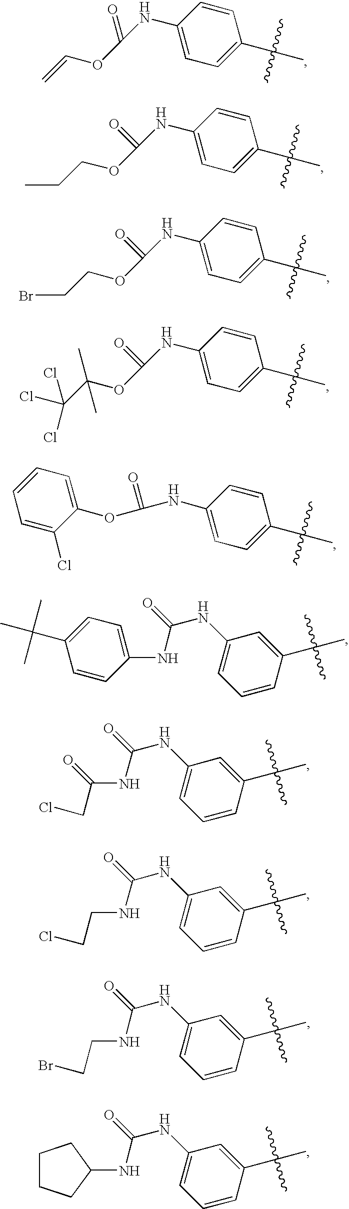

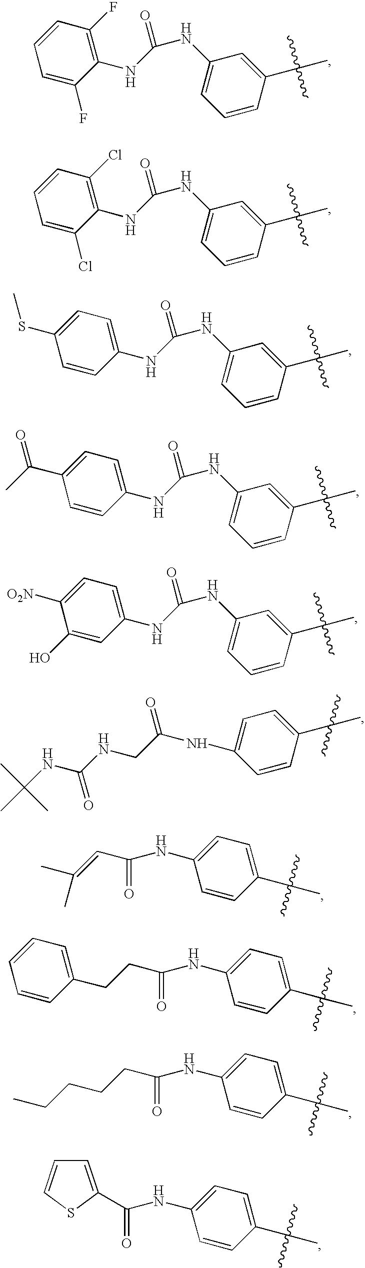

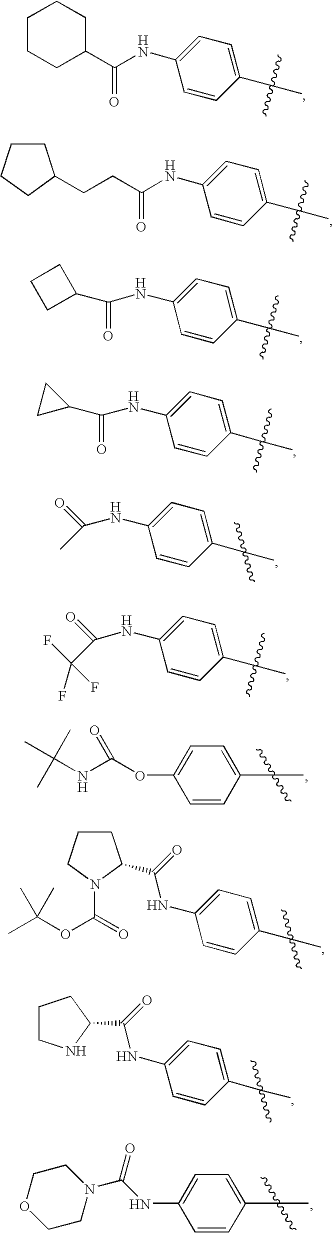

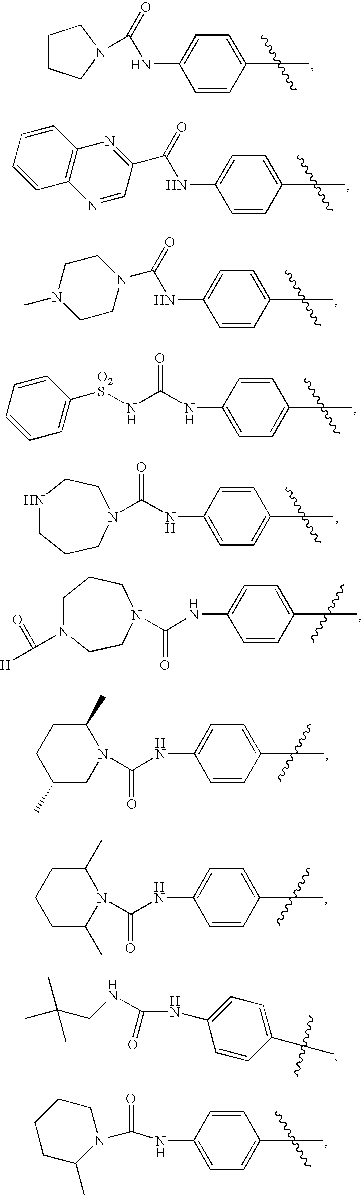

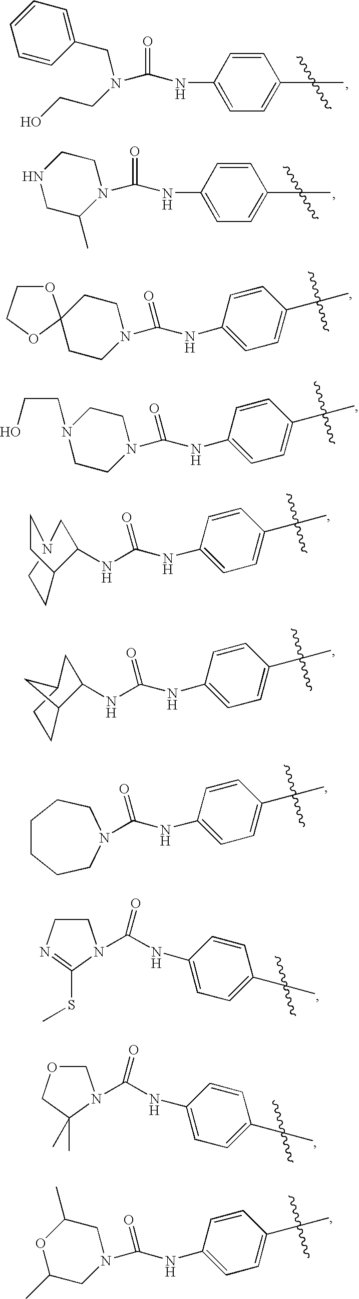

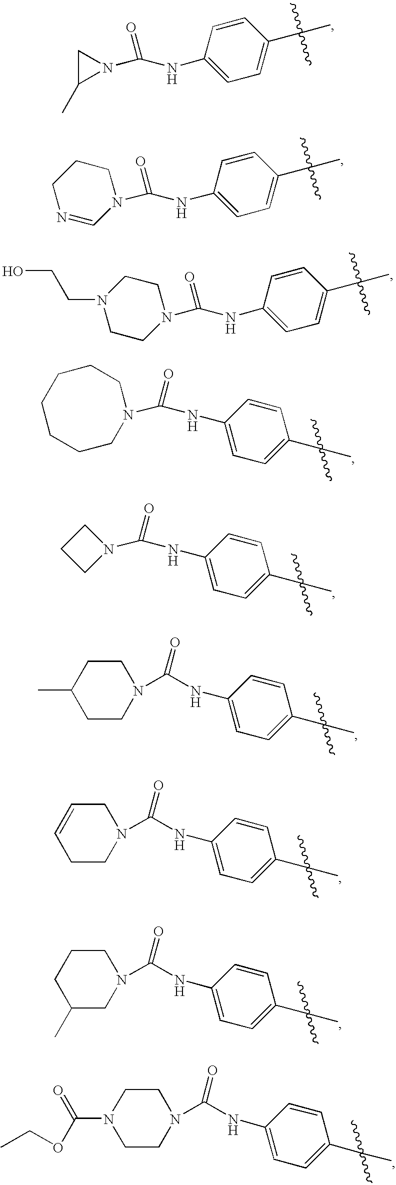

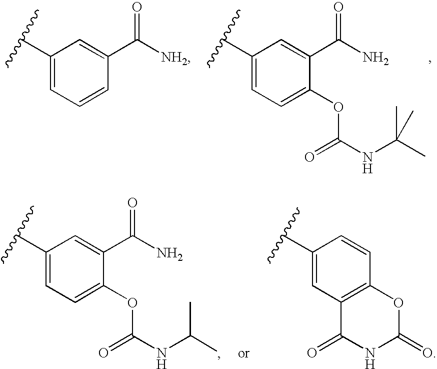

In some embodiments, A is alkyl substituted phenyl. In one embodiment, A is ethyl substituted phenyl, 4-t-butylphenyl, 4-methanesulfonylphenyl, 4-N,N-diethylaminophenyl and B is 4-[2-thiobenzimidazolone], 4-[2-(trifluoromethyl)benzimidazole] or N-t-butylcarbamoyl-4-aminophenyl.

In one embodiment, compounds of Formula I are 7-(2-{4-[2-(4-ethylphenyl)-1H-benzimidazol-4-yl]piperazin-1-yl}ethoxy)-1H-benzimidazol-2-ylcyanamide; ethyl 4-(2-{4-[2-(4-tert-butylphenyl)-1H-benzimidazol-4-yl]piperazin-1-yl}ethoxy)-2-imino-2,3-dihydro-1H-benzimidazole-1-carboxylate; 4-(2-{4-[2-(4-tert-butylphenyl)-1H-benzimidazol-4-yl]piperazin-1-yl}ethoxy)-1-propionyl-1,3-dihydro-2H-benzimidazol-2-imine; 4-(2-{4-[2-(4-tert-butylphenyl)-1H-benzimidazol-4-yl]piperazin-1-yl}ethoxy)-1-(2,2-dimethylpropanoyl)-1,3-dihydro-2H-benzimidazol-2-imine; 3-(4-{4-[4-(2-{[2-(trifluoromethyl)-1H-benzimidazol-4-yl]oxy}ethyl)piperazin-1-yl]-1H-benzimidazol-2-yl}benzyl)phenol; 2-(aminocarbonyl)-4-(2-{4-[2-(4-tert-butylphenyl)-1H-benzimidazol-4-yl]piperazin-1-yl}ethoxy)phenyl isopropylcarbamate; 2-(aminocarbonyl)-4-(2-{4-[2-(4-ethylphenyl)-1H-benzimidazol-4-yl]piperazin-1-yl}ethoxy)phenyl isopropylcarbamate; 6-(2-{4-[2-(4-tert-butylphenyl)-1H-benzimidazol-4-yl]piperazin-1-yl}ethoxy)-2H-1,3-benzoxazine-2,4(3H)-dione; 4-(2-{4-[2-(4-ethylphenyl)-1H-benzimidazol-4-yl]piperazin-1-yl}ethoxy)phenol; N-benzyl-N′-[4-(2-{4-[2-(4-tert-butylphenyl)-1H-benzimidazol-4-yl]piperazin-1-yl}ethoxy)phenyl]-N-(2-hydroxyethyl)urea; N-[4-(2-{4-[2-(4-tert-butylphenyl)-1H-benzimidazol-4-yl]piperazin-1-yl}ethoxy)phenyl]-2-methylpiperazine-1-carboxamide; N-[4-(2-{4-[2-(4-tert-butylphenyl)-1H-benzimidazol-4-yl]piperazin-1-yl}ethoxy)phenyl]-N′-neopentylurea; N-[4-(2-{4-[2-(4-tert-butylphenyl)-1H-benzimidazol-4-yl]piperazin-1-yl}ethoxy)phenyl]-2,6-dimethylpiperidine-1-carboxamide; (2S,5S)-N-[4-(2-{4-[2-(4-tert-butylphenyl)-1H-benzimidazol-4-yl]piperazin-1-yl}ethoxy)phenyl]-2,5-dimethylpiperidine-1-carboxamide; 2-(aminocarbonyl)-4-(2-{4-[2-(4-ethylphenyl)-1H-benzimidazol-4-yl]piperazin-1-yl}ethoxy)phenyl tert-butylcarbamate; N-[4-(2-{4-[2-(4-tert-butylphenyl)-1H-benzimidazol-4-yl]piperazin-1-yl}ethoxy)phenyl]-4-formyl-1,4-diazepane-1-carboxamide; N-[4-(2-{4-[2-(4-tert-butylphenyl)-1H-benzimidazol-4-yl]piperazin-1-yl}ethoxy)phenyl]-1,4-diazepane-1-carboxamide; N-({[4-(2-{4-[2-(4-tert-butylphenyl)-1H-benzimidazol-4-yl]piperazin-1-yl}ethoxy)phenyl]amino}carbonyl)benzenesulfonamide; N-[4-(2-{4-[2-(4-tert-butylphenyl)-1H-benzimidazol-4-yl]piperazin-1-yl}ethoxy)phenyl]-4-methylpiperazine-1-carboxamide; 3-(2-{4-[2-(4-ethylphenyl)-1H-benzimidazol-4-yl]piperazin-1-yl}ethoxy)benzamide; 2-(4,5,6,7-tetrahydro-1-benzothien-3-yl)-4-[4-(2-{[2-(trifluoromethyl)-1H-benzimidazol-4-yl]oxy}ethyl)piperazin-1-yl]-1H-benzimidazole; 2-(5-isopropylthien-2-yl)-4-[4-(2-{[2-(trifluoromethyl)-1H-benzimidazol-4-yl]oxy}ethyl)piperazin-1-yl]-1H-benzimidazole; 4-(2-{4-[2-(4-tert-butylphenyl)-1H-benzimidazol-4-yl]piperazin-1-yl}ethoxy)-1,3-dihydro-2H-benzimidazol-2-imine; N-[4-(2-{4-[2-(4-tert-butylphenyl)-1H-benzimidazol-4-yl]piperazin-1-yl}ethoxy)phenyl]quinoxaline-2-carboxamide; N-[4-(2-{4-[2-(4-tert-butylphenyl)-1H-benzimidazol-4-yl]piperazin-1-yl}ethoxy)phenyl]thiophene-2-carboxamide; N-[4-(2-{4-[2-(4-tert-butylphenyl)-1H-benzimidazol-4-yl]piperazin-1-yl}ethoxy)phenyl]pyrrolidine-1-carboxamide; N-[4-(2-{4-[2-(4-tert-butylphenyl)-1H-benzimidazol-4-yl]piperazin-1-yl}ethoxy)phenyl]morpholine-4-carboxamide; 4-(2-{4-[2-(4-ethylphenyl)-1H-benzimidazol-4-yl]piperazin-1-yl}ethoxy)benzamide; N-[4-(2-{4-[2-(4-ethylphenyl)-1H-benzimidazol-4-yl]piperazin-1-yl}ethoxy)phenyl]-L-prolinamide; tert-butyl (2S)-2-({[4-(2-{4-[2-(4-ethylphenyl)-1H-benzimidazol-4-yl]piperazin-1-yl}ethoxy)phenyl]amino}carbonyl)pyrrolidine-1-carboxylate; 4-(2-{4-[2-(4-ethylphenyl)-1H-benzimidazol-4-yl]piperazin-1-yl}ethoxy)phenyl tert-butylcarbamate; 2-(5-tert-butylthien-3-yl)-4-[4-(2-{[2-(trifluoromethyl)-1H-benzimidazol-4-yl]oxy}ethyl)piperazin-1-yl]-1H-benzimidazole; 2-(5-ethylthien-3-yl)-4-[4-(2-{[2-(trifluoromethyl)-1H-benzimidazol-4-yl]oxy}ethyl)piperazin-1-yl]-1H-benzimidazole; N2-[(tert-butylamino)carbonyl]-N1-[4-(2-{4-[2-(4-ethylphenyl)-1H-benzimidazol-4-yl]piperazin-1-yl}ethoxy)phenyl]glycinamide; 5-(2-{4-[2-(4-ethylphenyl)-1H-benzimidazol-4-yl]piperazin-1-yl}ethoxy)-2-nitrophenol; 4-(2-{4-[2-(4-tert-butylphenyl)-1H-benzimidazol-4-yl]piperazin-1-yl}ethoxy)aniline; N-(4-tert-butylphenyl)-N′-[4-(2-{4-[2-(4-ethylphenyl)-1H-benzimidazol-4-yl]piperazin-1-yl}ethoxy)phenyl]urea; 5-(2-{4-[2-(4-ethylphenyl)-1H-benzimidazol-4-yl]piperazin-1-yl}ethoxy)-2-hydroxybenzamide; 2-(4-benzylphenyl)-4-[4-(2-{[2-(trifluoromethyl)-1H-benzimidazol-4-yl]oxy}ethyl)piperazin-1-yl]-1H-benzimidazole; 2-(5-tert-butylthien-2-yl)-4-[4-(2-{[2-(trifluoromethyl)-1H-benzimidazol-4-yl]oxy}ethyl)piperazin-1-yl]-1H-benzimidazole; N-(tert-butyl)-N′-[4-(2-{4-[2-(4-tert-butylphenyl)-1H-benzimidazol-4-yl]piperazin-1-yl}ethoxy)phenyl]urea; N-(tert-butyl)-N′-[3-(2-{4-[2-(4-ethylphenyl)-1H-benzimidazol-4-yl]piperazin-1-yl}ethoxy)phenyl]urea; 3-(2-{4-[2-(4-ethylphenyl)-1H-benzimidazol-4-yl]piperazin-1-yl}ethoxy)aniline; 2-(4-ethylphenyl)-4-{4-[2-(4-nitrophenoxy)ethyl]piperazin-1-yl}-1H-benzimidazole; 2-(4-ethylphenyl)-4-{4-[2-(3-nitrophenoxy)ethyl]piperazin-1-yl}-1H-benzimidazole; N-[4-(2-{4-[2-(4-ethylphenyl)-1H-benzimidazol-4-yl]piperazin-1-yl}ethoxy)phenyl]-2,2-dimethylpropanamide; N-[4-(2-{4-[2-(4-Ethyl-phenyl)-1H-benzoimidazol-4-yl]-piperazin-1-yl}-ethoxy)-phenyl]-2,2-dimethyl-propionamide; N-[4-(2-{4-[2-(4-ethylphenyl)-1H-benzimidazol-4-yl]piperazin-1-yl}ethoxy)phenyl]methanesulfonamide; N-[4-(2-{4-[2-(4-ethylphenyl)-1H-benzimidazol-4-yl]piperazin-1-yl}ethoxy)phenyl]-3,3-dimethylbutanamide; tert-butyl 4-(2-{4-[2-(4-ethylphenyl)-1H-benzimidazol-4-yl]piperazin-1-yl}ethoxy)phenylcarbamate; 4-ethyl-N-[4-(2-{4-[2-(4-ethylphenyl)-1H-benzimidazol-4-yl]piperazin-1-yl}ethoxy)phenyl]benzamide; neopentyl 4-(2-{4-[2-(4-ethylphenyl)-1H-benzimidazol-4-yl]piperazin-1-yl}ethoxy)phenylcarbamate; [4-(2-{4-[2-(4-Ethyl-phenyl)-1H-benzoimidazol-4-yl]-piperazin-1-yl}-ethoxy)-phenyl]-carbamic acid 2,2-dimethyl-propyl ester; 4-(2-{4-[2-(4-ethylphenyl)-1H-benzimidazol-4-yl]piperazin-1-yl}ethoxy)aniline; N-(tert-butyl)-N′-[4-(2-{4-[2-(4-ethylphenyl)-1H-benzimidazol-4-yl]piperazin-1-yl}ethoxy)phenyl]urea; 4-{2-[4-(2-phenyl-1H-benzimidazol-7-yl)piperazin-1-yl]ethoxy}-1,3-dihydro-2H-benzimidazole-2-thione; ethyl 4-({[4-(2-{4-[2-(4-tert-butylphenyl)-1H-benzimidazol-4-yl]piperazin-1-yl}ethoxy)phenyl]amino}carbonyl)piperazine-1-carboxylate; N-[4-(2-{4-[2-(4-tert-butylphenyl)-1H-benzimidazol-4-yl]piperazin-1-yl}ethoxy)phenyl]-3-methylpiperidine-1-carboxamide; 3,6-Dihydro-2H-pyridine-1-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-3,6-dihydropyridine-1 (2H)-carboxamide; N-[4-(2-{4-[2-(4-tert-butylphenyl)-1H-benzimidazol-4-yl]piperazin-1-yl}ethoxy)phenyl]-4-methylpiperidine-1-carboxamide; N-[4-(2-{4-[2-(4-tert-butylphenyl)-1H-benzimidazol-4-yl]piperazin-1-yl}ethoxy)phenyl]azetidine-1-carboxamide; N-[4-(2-{4-[2-(4-tert-butylphenyl)-1H-benzimidazol-4-yl]piperazin-1-yl}ethoxy)phenyl]azocane-1-carboxamide; N-[4-(2-{4-[2-(4-tert-butylphenyl)-1H-benzimidazol-4-yl]piperazin-1-yl}ethoxy)phenyl]-4-(2-hydroxyethyl)piperazine-1-carboxamide; N-[4-(2-{4-[2-(4-tert-butylphenyl)-1H-benzimidazol-4-yl]piperazin-1-yl}ethoxy)phenyl]-5,6-dihydropyrimidine-1 (4H)-carboxamide; N-[4-(2-{4-[2-(4-tert-butylphenyl)-1H-benzimidazol-4-yl]piperazin-1-yl}ethoxy)phenyl]-2-methylaziridine-1-carboxamide; 2,6-Dimethyl-morpholine-4-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-2,6-dimethylmorpholine-4-carboxamide; N-[4-(2-{4-[2-(4-tert-butylphenyl)-1H-benzimidazol-4-yl]piperazin-1-yl}ethoxy)phenyl]-4,4-dimethyl-1,3-oxazolidine-3-carboxamide; N-[4-(2-{4-[2-(4-tert-butylphenyl)-1H-benzimidazol-4-yl]piperazin-1-yl}ethoxy)phenyl]-2-(methylthio)-4,5-dihydro-1H-imidazole-1-carboxamide; N-[4-(2-{4-[2-(4-tert-butylphenyl)-1H-benzimidazol-4-yl]piperazin-1-yl}ethoxy)phenyl]azepane-1-carboxamide; N-[(1R,2S,4S)-bicyclo[2.2.1]hept-2-yl]-N′-[4-(2-{4-[2-(4-tert-butylphenyl)-1H-benzimidazol-4-yl]piperazin-1-yl}ethoxy)phenyl]urea; N-1-azabicyclo[2.2.2]oct-3-yl-N′-[4-(2-{4-[2-(4-tert-butylphenyl)-1H-benzimidazol-4-yl]piperazin-1-yl}ethoxy)phenyl]urea; N-[4-(2-{4-[2-(4-tert-butylphenyl)-1H-benzimidazol-4-yl]piperazin-1-yl}ethoxy)phenyl]-2-methylpiperidine-1-carboxamide; N-[4-(2-{4-[2-(4-tert-butylphenyl)-1H-benzimidazol-4-yl]piperazin-1-yl}ethoxy)phenyl]-N′-[4-(2-hydroxyethyl)piperazin-1-yl]urea; 1,4-Dioxa-8-aza-spiro[4.5]decane-8-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-1,4-dioxa-8-azaspiro[4.5]decane-8-carboxamide; N-azepan-1-yl-N′-[4-(2-{4-[2-(4-tert-butylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-2,2,2-trifluoroacetamide; N-[4-(2-{4-[2-(4-ethylphenyl)-1H-benzimidazol-4-yl]piperazin-1-yl}ethoxy)phenyl]acetamide; N-[4-(2-{4-[2-(4-ethylphenyl)-1H-benzimidazol-4-yl]piperazin-1-yl}ethoxy)phenyl]cyclopropanecarboxamide; N-[4-(2-{4-[2-(4-ethylphenyl)-1H-benzimidazol-4-yl]piperazin-1-yl}ethoxy)phenyl]cyclobutanecarboxamide; 3-cyclopentyl-N-[4-(2-{4-[2-(4-ethylphenyl)-1H-benzimidazol-4-yl]piperazin-1-yl}ethoxy)phenyl]propanamide; N-[4-(2-{4-[2-(4-ethylphenyl)-1H-benzimidazol-4-yl]piperazin-1-yl}ethoxy)phenyl]cyclohexanecarboxamide; N-[4-(2-{4-[2-(4-ethylphenyl)-1H-benzimidazol-4-yl]piperazin-1-yl}ethoxy)phenyl]thiophene-2-carboxamide; N-[4-(2-{4-[2-(4-ethylphenyl)-1H-benzimidazol-4-yl]piperazin-1-yl}ethoxy)phenyl]hexanamide; N-[4-(2-{4-[2-(4-ethylphenyl)-1H-benzimidazol-4-yl]piperazin-1-yl}ethoxy)phenyl]-3-phenylpropanamide; N-[4-(2-{4-[2-(4-ethylphenyl)-1H-benzimidazol-4-yl]piperazin-1-yl}ethoxy)phenyl]-3-methylbut-2-enamide; N-(4-acetylphenyl)-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-[4-(methylthio)phenyl]urea; N-(2,6-dichlorophenyl)-N′-[4-(2-{4-[2-(4-ethylphenyl)-1H-benzimidazol-4-yl]piperazin-1-yl}ethoxy)phenyl]urea; N-(2,6-difluorophenyl)-N′-[4-(2-{4-[2-(4-ethylphenyl)-1H-benzimidazol-4-yl]piperazin-1-yl}ethoxy)phenyl]urea; N-cyclopentyl-N′-[4-(2-{4-[2-(4-ethylphenyl)-1H-benzimidazol-4-yl]piperazin-1-yl}ethoxy)phenyl]urea; N-(2-bromoethyl)-N′-[4-(2-{4-[2-(4-ethylphenyl)-1H-benzimidazol-4-yl]piperazin-1-yl}ethoxy)phenyl]urea; N-(2-chloroethyl)-N′-[4-(2-{4-[2-(4-ethylphenyl)-1H-benzimidazol-4-yl]piperazin-1-yl}ethoxy)phenyl]urea; 2-chloro-N-({[4-(2-{4-[2-(4-ethylphenyl)-1H-benzimidazol-4-yl]piperazin-1-yl}ethoxy)phenyl]amino}carbonyl)acetamide; N-(tert-butyl)-N′-[4-(2-{4-[2-(4-ethylphenyl)-1H-benzimidazol-4-yl]piperazin-1-yl}ethoxy)-2-fluorophenyl]urea; N-(tert-butyl)-N′-[4-(2-{4-[2-(4-ethylphenyl)-1H-benzimidazol-4-yl]piperazin-1-yl}ethoxy)-2-methylphenyl]urea; N-(tert-butyl)-N′-[2-chloro-4-(2-{4-[2-(4-ethylphenyl)-1H-benzimidazol-4-yl]piperazin-1-yl}ethoxy)phenyl]urea; [4-(2-{4-[2-(4-ethylphenyl)-1H-benzimidazol-4-yl]piperazin-1-yl}ethoxy)-2-methylphenyl]amine; [4-(2-{4-[2-(4-ethylphenyl)-1H-benzimidazol-4-yl]piperazin-1-yl}ethoxy)-2-fluorophenyl]amine; [2-chloro-4-(2-{4-[2-(4-ethylphenyl)-1H-benzimidazol-4-yl]piperazin-1-yl}ethoxy)phenyl]amine; 2-(4-ethylphenyl)-4-{4-[2-(3-fluoro-4-nitrophenoxy)ethyl]piperazin-1-yl}-1H-benzimidazole; 2-(4-ethylphenyl)-4-{4-[2-(3-methyl-4-nitrophenoxy)ethyl]piperazin-1-yl}-1H-benzimidazole; 2-chlorophenyl [4-(2-{4-[2-(4-ethylphenyl)-1H-benzimidazol-4-yl]piperazin-1-yl}ethoxy)phenyl]carbamate; 2,2,2-trichloro-1,1-dimethylethyl [4-(2-{4-[2-(4-ethylphenyl)-1H-benzimidazol-4-yl]piperazin-1-yl}ethoxy)phenyl]carbamate; 2-bromoethyl [4-(2-{4-[2-(4-ethylphenyl)-1H-benzimidazol-4-yl]piperazin-1-yl}ethoxy)phenyl]carbamate; propyl [4-(2-{4-[2-(4-ethylphenyl)-1H-benzimidazol-4-yl]piperazin-1-yl}ethoxy)phenyl]carbamate; vinyl [4-(2-{4-[2-(4-ethylphenyl)-1H-benzimidazol-4-yl]piperazin-1-yl}ethoxy)phenyl]carbamate; allyl [4-(2-{4-[2-(4-ethylphenyl)-1H-benzimidazol-4-yl]piperazin-1-yl}ethoxy)phenyl]carbamate; N-[4-(2-{4-[2-(4-ethylphenyl)-1H-benzimidazol-4-yl]piperazin-1-yl}ethoxy)phenyl]adamantane-1-carboxamide; N-[4-(2-{4-[2-(4-ethylphenyl)-1H-benzimidazol-4-yl]piperazin-1-yl}ethoxy)phenyl]isonicotinamide; N-[4-(2-{4-[2-(4-ethylphenyl)-1H-benzimidazol-4-yl]piperazin-1-yl}ethoxy)phenyl]nicotinamide; N-[4-(2-{4-[2-(4-ethylphenyl)-1H-benzimidazol-4-yl]piperazin-1-yl}ethoxy)phenyl]-2-methoxybenzamide; N-[4-(2-{4-[2-(4-ethylphenyl)-1H-benzimidazol-4-yl]piperazin-1-yl}ethoxy)phenyl]-2,6-difluorobenzamide; N-[4-(2-{4-[2-(4-ethylphenyl)-1H-benzimidazol-4-yl]piperazin-1-yl}ethoxy)phenyl]cyclopentanecarboxamide; N-[4-(2-{4-[2-(4-ethylphenyl)-1H-benzimidazol-4-yl]piperazin-1-yl}ethoxy)phenyl]-2-(trifluoromethyl)benzamide; 2-ethyl-N-[4-(2-{4-[2-(4-ethylphenyl)-1H-benzimidazol-4-yl]piperazin-1-yl}ethoxy)phenyl]butanamide; N-[4-(2-{4-[2-(4-ethylphenyl)-1H-benzimidazol-4-yl]piperazin-1-yl}ethoxy)phenyl]-2-methylbenzamide; 2,6-dichloro-N-[4-(2-{4-[2-(4-ethylphenyl)-1H-benzimidazol-4-yl]piperazin-1-yl}ethoxy)phenyl]benzamide; N-[4-(2-{4-[2-(4-ethylphenyl)-1H-benzimidazol-4-yl]piperazin-1-yl}ethoxy)phenyl]-2-(2-thienyl)acetamide; N-[4-(2-{4-[2-(4-ethylphenyl)-1H-benzimidazol-4-yl]piperazin-1-yl}ethoxy)phenyl]-2-furamide; N-[4-(2-{4-[2-(4-ethylphenyl)-1H-benzimidazo-4-yl]piperazin-1-yl}ethoxy)phenyl]-3-methylbutanamide; (2E)-N-[4-(2-{4-[2-(4-ethylphenyl)-1H-benzimidazol-4-yl]piperazin-1-yl}ethoxy)phenyl]but-2-enamide; N-[4-(2-{4-[2-(4-ethylphenyl)-1H-benzimidazol-4-yl]piperazin-1-yl}ethoxy)phenyl]acrylamide; N-[4-(2-{4-[2-(4-ethylphenyl)-1H-benzimidazol-4-yl]piperazin-1-yl}ethoxy)phenyl]propanamide; N-[4-(2-{4-[2-(4-ethylphenyl)-1H-benzimidazol-4-yl]piperazin-1-yl}ethoxy)phenyl]thiophene-2-sulfonamide; N-[4-(2-{4-[2-(4-ethylphenyl)-1H-benzimidazol-4-yl]piperazin-1-yl}ethoxy)phenyl]-4-fluorobenzenesulfonamide; N-[4-(2-{4-[2-(4-ethylphenyl)-1H-benzimidazol-4-yl]piperazin-1-yl}ethoxy) phenyl]-4-methoxybenzenesulfonamide; N-[4-(2-{4-[2-(4-ethylphenyl)-1H-benzimidazol-4-yl]piperazin-1-yl}ethoxy)phenyl]-2-methylbenzenesulfonamide; 4-tert-butyl-N-[4-(2-{4-[2-(4-ethylphenyl)-1H-benzimidazol-4-yl]piperazin-1-yl}ethoxy)phenyl]benzenesulfonamide; N-[4-(2-{4-[2-(4-ethylphenyl)-1H-benzimidazol-4-yl]piperazin-1-yl}ethoxy)phenyl]-4-nitrobenzenesulfonamide; N-[4-(2-{4-[2-(4-ethylphenyl)-1H-benzimidazol-4-yl]piperazin-1-yl}ethoxy)phenyl]-3-nitrobenzenesulfonamide; N-[4-(2-{4-[2-(4-ethylphenyl)-1H-benzimidazol-4-yl]piperazin-1-yl}ethoxy)phenyl]-2-nitrobenzenesulfonamide; N-[4-(2-{4-[2-(4-ethylphenyl)-1H-benzimidazol-4-yl]piperazin-1-yl}ethoxy)phenyl]benzenesulfonamide; N-[4-(2-{4-[2-(4-ethylphenyl)-1H-benzimidazol-4-yl]piperazin-1-yl}ethoxy)phenyl]butane-1-sulfonamide; 3-chloro-N-[4-(2-{4-[2-(4-ethylphenyl)-1H-benzimidazol-4-yl]piperazin-1-yl}ethoxy)phenyl]propane-1-sulfonamide; N-[4-(2-{4-[2-(4-ethylphenyl)-1H-benzimidazol-4-yl]piperazin-1-yl}ethoxy)phenyl]propane-2-sulfonamide; N-[4-(2-{4-[2-(4-ethylphenyl)-1H-benzimidazol-4-yl]piperazin-1-yl}ethoxy)phenyl]propane-1-sulfonamide; 2-chloro-N-[4-(2-{4-[2-(4-ethylphenyl)-1H-benzimidazol-4-yl]piperazin-1-yl}ethoxy)phenyl]ethanesulfonamide; N-[4-(2-{4-[2-(4-ethylphenyl)-1H-benzimidazol-4-yl]piperazin-1-yl}ethoxy)phenyl]ethanesulfonamide; N-[4-(2-{4-[2-(4-ethylphenyl)-1H-benzimidazol-4-yl]piperazin-1-yl}ethoxy)phenyl]-N′-(1,1,3,3-tetramethylbutyl)urea; N-[4-(2-{4-[2-(4-ethylphenyl)-1H-benzimidazol-4-yl]piperazin-1-yl}ethoxy)phenyl]-N′-(4-nitrophenyl)urea; N-[4-(2-{4-[2-(4-ethylphenyl)-1H-benzimidazol-4-yl]piperazin-1-yl}ethoxy)phenyl]-N′-(2-phenylethyl)urea; N-benzyl-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-(3-fluorophenyl)urea; N-[4-(2-{4-[2-(4-ethylphenyl)-1H-benzimidazol-4-yl]piperazin-1-yl}ethoxy)phenyl]-N′-(2-fluorophenyl)urea; N-[4-(2-{4-[2-(4-ethylphenyl)-1H-benzimidazol-4-yl]piperazin-1-yl}ethoxy)phenyl]-N′-(3-methylphenyl)urea; N-[4-(2-{4-[2-(4-ethylphenyl)-1H-benzimidazol-4-yl]piperazin-1-yl}ethoxy)phenyl]-N′-(2-methylphenyl)urea; N-(4-ethylphenyl)-N′-[4-(2-{4-[2-(4-ethylphenyl)-1H-benzimidazol-4-yl]piperazin-1-yl}ethoxy)phenyl]urea; N-cyclohexyl-N′-[4-(2-{4-[2-(4-ethylphenyl)-1H-benzimidazol-4-yl]piperazin-1-yl}ethoxy)phenyl]urea; N-allyl-N′-[4-(2-{4-[2-(4-ethylphenyl)-1H-benzimidazol-4-yl]piperazin-1-yl}ethoxy)phenyl]urea; ethyl ({[4-(2-{4-[2-(4-ethylphenyl)-1H-benzimidazol-4-yl]piperazin-1-yl}ethoxy)phenyl]amino}carbonyl)carbamate; N-butyl-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-isopropylurea; N-[4-(2-{4-[2-(4-ethylphenyl)-1H-benzimidazol-4-yl]piperazin-1-yl}ethoxy)phenyl]-N′-propylurea; N-ethyl-N′-[4-(2-{4-[2-(4-ethylphenyl)-1H-benzimidazol-4-yl]piperazin-1-yl}ethoxy)phenyl]urea; (3-{4-[4-(2-{[2-(trifluoromethyl)-1H-benzimidazol-4-yl]oxy}ethyl)piperazin-1-yl]-1H-benzimidazol-2-yl}phenyl)amine; 2-pyridin-4-yl-4-[4-(2-{[2-(trifluoromethyl)-1H-benzimidazol-4-yl]oxy}ethyl)piperazin-1-yl]-1H-benzimidazole; 2-(2,4-dimethoxyphenyl)-4-[4-(2-{[2-(trifluoromethyl)-1H-benzimidazol-4-yl]oxy}ethyl)piperazin-1-yl]-1H-benzimidazole; 2-(2,4-dichlorophenyl)-4-[4-(2-{[2-(trifluoromethyl)-1H-benzimidazol-4-yl]oxy}ethyl)piperazin-1-yl]-1H-benzimidazole; 2-methoxy-5-{4-[4-(2-{[2-(trifluoromethyl)-1H-benzimidazol-4-yl]oxy}ethyl)piperazin-1-yl]-1H-benzimidazol-2-yl}phenol; 2-(2,4-dimethylphenyl)-4-[4-(2-{[2-(trifluoromethyl)-1H-benzimidazol-4-yl]oxy}ethyl)piperazin-1-yl]-1H-benzimidazole; 2-methyl-5-{4-[4-(2-{[2-(trifluoromethyl)-1H-benzimidazol-4-yl]oxy}ethyl)piperazin-1-yl]-1H-benzimidazol-2-yl}phenol; 2-(trifluoromethyl)-4-{2-[4-(2-{4-[(trifluoromethyl)thio]phenyl}-1H-benzimidazol-4-yl)piperazin-1-yl]ethoxy}-1H-benzimidazole; 2-(4-fluorophenyl)-4-[4-(2-{[2-(trifluoromethyl)-1H-benzimidazol-4-yl]oxy}ethyl)piperazin-1-yl]-1H-benzimidazole; (4-{4-[4-(2-{[2-(trifluoromethyl)-1H-benzimidazol-4-yl]oxy}ethyl)piperazin-1-yl]-1H-benzimidazol-2-yl}phenyl)amine; 2-[4-(trifluoromethoxy)phenyl]-4-[4-(2-{[2-(trifluoromethyl)-1H-benzimidazol-4-yl]oxy}ethyl)piperazin-1-yl]-1H-benzimidazole; 2-(4-cyclohexylphenyl)-4-[4-(2-{[2-(trifluoromethyl)-1H-benzimidazol-4-yl]oxy}ethyl)piperazin-1-yl]-1H-benzimidazole; 2-[4-(methylthio)phenyl]-4-[4-(2-{[2-(trifluoromethyl)-1H-benzimidazol-4-yl]oxy}ethyl)piperazin-1-yl]-1H-benzimidazole; 2-[4-(benzyloxy)phenyl]-4-[4-(2-{[2-(trifluoromethyl)-1H-benzimidazol-4-yl]oxy}ethyl)piperazin-1-yl]-1H-benzimidazole; 2-(4-iodophenyl)-4-[4-(2-{[2-(trifluoromethyl)-1H-benzimidazol-4-yl]oxy}ethyl)piperazin-1-yl]-1H-benzimidazole; 4-{4-[4-(2-{[2-(trifluoromethyl)-1H-benzimidazol-4-yl]oxy}ethyl)piperazin-1-yl]-1H-benzimidazol-2-yl}benzenesulfonamide; 2-(4-propoxyphenyl)-4-[4-(2-{[2-(trifluoromethyl)-1H-benzimidazol-4-yl]oxy}ethyl)piperazin-1-yl]-1H-benzimidazole; 2-[4-(hexyloxy)phenyl]-4-[4-(2-{[2-(trifluoromethyl)-1H-benzimidazol-4-yl]oxy}ethyl)piperazin-1-yl]-1H-benzimidazole; 2-(4-propylphenyl)-4-[4-(2-{[2-(trifluoromethyl)-1H-benzimidazol-4-yl]oxy}ethyl)piperazin-1-yl]-1H-benzimidazole; 2-[4-(methylsulfonyl)phenyl]-4-[4-(2-{[2-(trifluoromethyl)-1H-benzimidazol-4-yl]oxy}ethyl)piperazin-1-yl]-1H-benzimidazole; 2-(4-hexylphenyl)-4-[4-(2-{[2-(trifluoromethyl)-1H-benzimidazol-4-yl]oxy}ethyl)piperazin-1-yl]-1H-benzimidazole; 2-[4-(heptyloxy)phenyl]-4-[4-(2-{[2-(trifluoromethyl)-1H-benzimidazol-4-yl]oxy}ethyl)piperazin-1-yl]-1H-benzimidazole; N-butyl-4-{4-[4-(2-{[2-(trifluoromethyl)-1H-benzimidazol-4-yl]oxy}ethyl)piperazin-1-yl]-1H-benzimidazol-2-yl}aniline; Phenyl-[4-(4-{4-[2-(2-trifluoromethyl-1H-benzoimidazol-4-yloxy)-ethyl]-piperazin-1-yl}-1H-benzoimidazol-2-yl)-phenyl]-methanone; phenyl(4-{4-[4-(2-{[2-(trifluoromethyl)-1H-benzimidazol-4-yl]oxy}ethyl)piperazin-1-yl]-1H-benzimidazol-2-yl}phenyl)methanone; 2-(trifluoromethyl)-4-(2-{4-[2-(4-vinylphenyl)-1H-benzimidazol-4-yl]piperazin-1-yl}ethoxy)-1H-benzimidazole; 2-(4-pentylphenyl)-4-[4-(2-{[2-(trifluoromethyl)-1H-benzimidazol-4-yl]oxy}ethyl)piperazin-1-yl]-1H-benzimidazole; 2-(3-thienyl)-4-[4-(2-{[2-(trifluoromethyl)-1H-benzimidazol-4-yl]oxy}ethyl)piperazin-1-yl]-1H-benzimidazole; 2-(4-butylphenyl)-4-[4-(2-{[2-(trifluoromethyl)-1H-benzimidazol-4-yl]oxy}ethyl)piperazin-1-yl]-1H-benzimidazole; 4-(4-{4-[4-(2-{[2-(trifluoromethyl)-1H-benzimidazol-4-yl]oxy}ethyl)piperazin-1-yl]-1H-benzimidazol-2-yl}phenoxy)phenol; 2-[5-(methylthio)-2-thienyl]-4-[4-(2-{[2-(trifluoromethyl)-1H-benzimidazol-4-yl]oxy}ethyl)piperazin-1-yl]-1H-benzimidazole; 2-(4-phenoxyphenyl)-4-[4-(2-{[2-(trifluoromethyl)-1H-benzimidazol-4-yl]oxy}ethyl)piperazin-1-yl]-1H-benzimidazole; 2-cyclohexyl-4-[4-(2-{[2-(trifluoromethyl)-1H-benzimidazol-4-yl]oxy}ethyl)piperazin-1-yl]-1H-benzimidazole; 2-(5-nitro-2-thienyl)-4-[4-(2-{[2-(trifluoromethyl)-1H-benzimidazol-4-yl]oxy}ethyl)piperazin-1-yl]-1H-benzimidazole; 2-(4-butoxyphenyl)-4-[4-(2-{[2-(trifluoromethyl)-1H-benzimidazol-4-yl]oxy}ethyl)piperazin-1-yl]-1H-benzimidazole; 2-(4-nitrophenyl)-4-[4-(2-{[2-(trifluoromethyl)-1H-benzimidazol-4-yl]oxy}ethyl)piperazin-1-yl]-1H-benzimidazole; 2-(4-tert-butylphenyl)-4-[4-(2-{[2-(trifluoromethyl)-1H-benzimidazol-4-yl]oxy}ethyl)piperazin-1-yl]-1H-benzimidazole; 2-(4-tert-butylphenyl)-4-[4-(2-{[2-(trifluoromethyl)-1H-benzimidazol-4-yl]oxy}ethyl)piperazin-1-yl]-1H-benzimidazole; 2-[5-(4-fluorophenyl)-2-thienyl]-4-[4-(2-{[2-(trifluoromethyl)-1H-benzimidazol-4-yl]oxy}ethyl)piperazin-1-yl]-1H-benzimidazole; 2-[5-(4-methoxyphenyl)-2-thienyl]-4-[4-(2-{[2-(trifluoromethyl)-1H-benzimidazol-4-yl]oxy}ethyl)piperazin-1-yl]-1H-benzimidazole; 2-(4-ethoxyphenyl)-4-[4-(2-{[2-(trifluoromethyl)-1H-benzimidazol-4-yl]oxy}ethyl)piperazin-1-yl]-1H-benzimidazole; 2-(4-methoxyphenyl)-4-[4-(2-{[2-(trifluoromethyl)-1H-benzimidazol-4-yl]oxy}ethyl)piperazin-1-yl]-1H-benzimidazole; 2-[4-(1H-pyrrol-1-yl)phenyl]-4-[4-(2-{[2-(trifluoromethyl)-1H-benzimidazol-4-yl]oxy}ethyl)piperazin-1-yl]-1H-benzimidazole; N,N-diethyl-4-{4-[4-(2-{[2-(trifluoromethyl)-1H-benzimidazol-4-yl]oxy}ethyl)piperazin-1-yl]-1H-benzimidazol-2-yl}aniline; 2-{5-[1-methyl-3-(trifluoromethyl)-1H-pyrazol-5-yl]-2-thienyl}-4-[4-(2-{[2-(trifluoromethyl)-1H-benzimidazol-4-yl]oxy}ethyl)piperazin-1-yl]-1H-benzimidazole; 4-{4-[4-(2-{[2-(trifluoromethyl)-1H-benzimidazol-4-yl]oxy}ethyl)piperazin-1-yl]-1H-benzimidazol-2-yl}benzonitrile; N-methyl-4-{4-[4-(2-{[2-(trifluoromethyl)-1H-benzimidazol-4-yl]oxy}ethyl)piperazin-1-yl]-1H-benzimidazol-2-yl}aniline; 2-(5-methyl-2-thienyl)-4-[4-(2-{[2-(trifluoromethyl)-1H-benzimidazol-4-yl]oxy}ethyl)piperazin-1-yl]-1H-benzimidazole; 2-(4-bromophenyl)-4-[4-(2-{[2-(trifluoromethyl)-1H-benzimidazol-4-yl]oxy}ethyl)piperazin-1-yl]-1H-benzimidazole; 2-biphenyl-4-yl-4-[4-(2-{[2-(trifluoromethyl)-1H-benzimidazol-4-yl]oxy}ethyl)piperazin-1-yl]-1H-benzimidazole; 2-(5-pyridin-2-yl-2-thienyl)-4-[4-(2-{[2-(trifluoromethyl)-1H-benzimidazol-4-yl]oxy}ethyl)piperazin-1-yl]-1H-benzimidazole; 2-[4-(pentyloxy)phenyl]-4-[4-(2-{[2-(trifluoromethyl)-1H-benzimidazol-4-yl]oxy}ethyl)piperazin-1-yl]-1H-benzimidazole; 2-(4-ethylphenyl)-4-[4-(2-{[2-(trifluoromethyl)-1H-benzimidazol-4-yl]oxy}ethyl)piperazin-1-yl]-1H-benzimidazole; 2-(5-bromo-2-thienyl)-4-[4-(2-{[2-(trifluoromethyl)-1H-benzimidazol-4-yl]oxy}ethyl)piperazin-1-yl]-1H-benzimidazole; 2-(4-isopropylphenyl)-4-[4-(2-{[2-(trifluoromethyl)-1H-benzimidazol-4-yl]oxy}ethyl)piperazin-1-yl]-1H-benzimidazole; N-(4-{4-[4-(2-{[2-(trifluoromethyl)-1H-benzimidazol-4-yl]oxy}ethyl)piperazin-1-yl]-1H-benzimidazol-2-yl}phenyl)acetamide; 2-(4-methylphenyl)-4-[4-(2-{[2-(trifluoromethyl)-1H-benzimidazol-4-yl]oxy}ethyl)piperazin-1-yl]-1H-benzimidazole; 2-(5-chloro-2-thienyl)-4-[4-(2-{[2-(trifluoromethyl)-1H-benzimidazol-4-yl]oxy}ethyl)piperazin-1-yl]-1H-benzimidazole; 2-(4-chlorophenyl)-4-[4-(2-{[2-(trifluoromethyl)-1H-benzimidazol-4-yl]oxy}ethyl)piperazin-1-yl]-1H-benzimidazole; 3-[4-(4-{4-[2-(2-Trifluoromethyl-1H-benzoimidazol-4-yloxy)-ethyl]-piperazin-1-yl)}-1H-benzoimidazol-2-yl)-phenoxy]-phenol; 3-(4-{4-[4-(2-{[2-(trifluoromethyl)-1H-benzimidazol-4-yl]oxy}ethyl)piperazin-1-yl]-1H-benzimidazol-2-yl}phenoxy)phenol; 2-(2-thienyl)-4-[4-(2-{[2-(trifluoromethyl)-1H-benzimidazol-4-yl]oxy}ethyl)piperazin-1-yl]-1H-benzimidazole; N,N-dimethyl-4-{4-[4-(2-{[2-(trifluoromethyl)-1H-benzimidazol-4-yl]oxy}ethyl)piperazin-1-yl]-1H-benzimidazol-2-yl}aniline; 4-(2-{4-[2-(3-thienyl)-1H-benzimidazol-4-yl]piperazin-1-yl}ethoxy)-1,3-dihydro-2H-benzimidazole-2-thione; 4-(2-{4-[2-(1-naphthyl)-1H-benzimidazol-4-yl]piperazin-1-yl}ethoxy)-1,3-dihydro-2H-benzimidazole-2-thione; 4-(2-{4-[2-(2-naphthyl)-1H-benzimidazol-4-yl]piperazin-1-yl}ethoxy)-1,3-dihydro-2H-benzimidazole-2-thione; 4-(2-{4-[2-(3-aminophenyl)-1H-benzimidazol-4-yl]piperazin-1-yl}ethoxy)-1,3-dihydro-2H-benzimidazole-2-thione; 4-(2-{4-[2-(3-hydroxy-4-methoxyphenyl)-1H-benzimidazol-4-yl]piperazin-1-yl}ethoxy)-1,3-dihydro-2H-benzimidazole-2-thione; 4-(2-{4-[2-(3-hydroxy-4-methylphenyl)-1H-benzimidazol-4-yl]piperazin-1-yl}ethoxy)-1,3-dihydro-2H-benzimidazole-2-thione; 4-[2-(4-{2-[4-(trifluoromethyl)phenyl]-1H-benzimidazol-4-yl}piperazin-1-yl)ethoxy]-1,3-dihydro-2H-benzimidazole-2-thione; 4-[2-(4-{2-[3-(trifluoromethyl)phenyl]-1H-benzimidazol-4-yl}piperazin-1-yl)ethoxy]-1,3-dihydro-2H-benzimidazole-2-thione; 4-[2-(4-{2-[3,5-bis(trifluoromethyl)phenyl]-1H-benzimidazol-4-yl}piperazin-1-yl)ethoxy]-1,3-dihydro-2H-benzimidazole-2-thione; 4-(2-{4-[2-(4-ethylphenyl)-1H-benzimidazol-4-yl]piperazin-1-yl}ethoxy)-1,3-dihydro-2H-benzimidazole-2-thione; 4-(2-{4-[2-(4-phenoxyphenyl)-1H-benzimidazol-4-yl]piperazin-1-yl}ethoxy)-1,3-dihydro-2H-benzimidazole-2-thione; 4-(2-{4-[2-(2,4-dimethylphenyl)-1H-benzimidazol-4-yl]piperazin-1-yl}ethoxy)-1,3-dihydro-2H-benzimidazole-2-thione; 4-(2-{4-[2-(3,4-dimethoxyphenyl)-1H-benzimidazol-4-yl]piperazin-1-yl}ethoxy)-1,3-dihydro-2H-benzimidazole-2-thione; 4-(2-{4-[2-(3,5-dimethylphenyl)-1H-benzimidazol-4-yl]piperazin-1-yl}ethoxy)-1,3-dihydro-2H-benzimidazole-2-thione; 4-(2-{4-[2-(3,5-difluorophenyl)-1H-benzimidazol-4-yl]piperazin-1-yl}ethoxy)-1,3-dihydro-2H-benzimidazole-2-thione; 4-(2-{4-[2-(3-methylphenyl)-1H-benzimidazol-4-yl]piperazin-1-yl}ethoxy)-1,3-dihydro-2H-benzimidazole-2-thione; 4-(2-{4-[2-(3-bromophenyl)-1H-benzimidazol-4-yl]piperazin-1-yl}ethoxy)-1,3-dihydro-2H-benzimidazole-2-thione; 4-(2-{4-[2-(2,4-dichlorophenyl)-1H-benzimidazol-4-yl]piperazin-1-yl}ethoxy)-1,3-dihydro-2H-benzimidazole-2-thione; 4-(2-{4-[2-(4-bromophenyl)-1H-benzimidazol-4-yl]piperazin-1-yl}ethoxy)-1,3-dihydro-2H-benzimidazole-2-thione; 4-(2-{4-[2-(2,3,6-trifluorophenyl)-1H-benzimidazol-4-yl]piperazin-1-yl}ethoxy)-1,3-dihydro-2H-benzimidazole-2-thione; 4-(2-{4-[2-(diphenylmethyl)-1H-benzimidazol-4-yl]piperazin-1-yl}ethoxy)-1,3-dihydro-2H-benzimidazole-2-thione; 4-(2-{4-[2-(2,2-diphenylethyl)-1H-benzimidazol-4-yl]piperazin-1-yl}ethoxy)-1,3-dihydro-2H-benzimidazole-2-thione; 4-(2-{4-[2-(2,4-dimethoxyphenyl)-1H-benzimidazol-4-yl]piperazin-1-yl}ethoxy)-1,3-dihydro-2H-benzimidazole-2-thione; 4-(2-{4-[2-(2,4,6-trimethoxyphenyl)-1H-benzimidazol-4-yl]piperazin-1-yl}ethoxy)-1,3-dihydro-2H-benzimidazole-2-thione; 4-(2-{4-[2-(4-methoxyphenyl)-1H-benzimidazol-4-yl]piperazin-1-yl}ethoxy)-1,3-dihydro-2H-benzimidazole-2-thione; 4-{2-[4-(2-pyridin-4-yl-1H-benzimidazol-4-yl)piperazin-1-yl]ethoxy}-1,3-dihydro-2H-benzimidazole-2-thione; 4-{2-[4-(2-pyridin-3-yl-1H-benzimidazol-4-yl)piperazin-1-yl]ethoxy}-1,3-dihydro-2H-benzimidazole-2-thione; 3-[4-(4-{2-[(2-thioxo-2,3-dihydro-1H-benzimidazol-4-yl)oxy]ethyl}piperazin-1-yl)-1H-benzimidazol-2-yl]benzonitrile; 4-[4-(4-{2-[(2-thioxo-2,3-dihydro-1H-benzimidazol-4-yl)oxy]ethyl}piperazin-1-yl)-1H-benzimidazol-2-yl]benzonitrile; 4-{2-[4-(2-pyridin-2-yl-1H-benzimidazol-4-yl)piperazin-1-yl]ethoxy}-1,3-dihydro-2H-benzimidazole-2-thione; or stereoisomers or pharmaceutically acceptable salts thereof.

The present invention also provides methods for modulating the activity of a Gonadotropin Releasing Hormone receptor, comprising contacting said receptor with an effective amount of a compound according to Formula I. In some embodiments, the method further comprises determining the activity of said receptor. The determination may be made before or after said contacting step.

The present invention also includes methods for treating a patient suspected of suffering from a condition associated with excessive Gonadotropin Releasing Hormone receptor activity, comprising the step of administering to the patient a therapeutically effective amount of a compound according to Formula I. Such conditions include prostate cancer, endometriosis, uterine fibroids, uterine cancer, breast cancer, ovarian cancer, testicular cancer, primary hirsutism, or LH surge.

The present invention also comprises pharmaceutical compositions comprising compounds of the above-described Formula I and a pharmaceutically acceptable carrier.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, such as sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

In one embodiment, the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

In certain embodiments, the present invention is directed to prodrugs. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

It is understood that the dosage, regimen and mode of administration of these compounds will vary according to the malady and the individual being treated and will be subject to the judgment of the medical practitioner involved. In one embodiment, the administration of one or more of the compounds herein begin at a low dose and be increased until the desired effects are achieved.

In one embodiment, the compounds of the present invention are administered in combination with an additional active agent.

In one embodiment, the additional active agent is selected from the group consisting of at least one of androgens, estrogens, progesterones, antiestrogens, antiprogestogens, testosterone, antiprogestogens, angiotensin-converting enzyme inhibitor (such as ENALAPRIL or CAPTOPRIL), angiotensin II-receptor antagonist (such as LOSARTAN), renin inhibitor, bisphosphonates (bisphosphonic acids), growth hormone secretagogues (such as MK-0677), 5a-reductase 2 inhibitor (such as finasteride or epristeride), a 5a-reductase 1 inhibitor (such as 4,7b-dimethyl-4-aza-5a-cholestan-3-one, 3-oxo-4-aza-4,7b-dimethyl-16b-(4-chlorophenoxy)-5a-androstane, and 3-oxo-4-aza-4,7b-dimethyl-16b-(phenoxy)-5a-androstane), dual inhibitors of 5a-reductase 1 and 5a-reductase 2 (such as 3-oxo-4-aza-17b-(2,5-trifluoromethylphenyl-carbamoyl)-5a-androstan), antiandrogens (such as flutamide, casodex and cyproterone acetate), alpha-1 blockers (such as prazosin, terazosin, doxazosin, tamsulosin, and alfuzosin), growth hormone, and luteinizing hormone releasing compounds (such as a peptide (including leuprorelin, gonadorelin, buserelin, triptorelin, goserelin, nafarelin, histrelin, deslorelin, meterlin and recirelin) or natural hormone or analog thereof).

For example, when used with compounds of the present invention: androgens, estrogens, progesterones, antiestrogens and antiprogestogens find use in the treatment of endometriosis, fibroids and in contraception; testosterone or other androgens or antiprogestogens find use in men as a contraceptive; angiotensin-converting enzyme inhibitors, angiotensin II-receptor antagonists, and renin inhibitor find use in the treatment of uterine fibroids; bisphosphonates (bisphosphonic acids) and growth hormone secretagogues find use in the treatment and prevention of disturbances of calcium, phosphate and bone metabolism, in particular, for the prevention of bone loss during therapy with the GnRH antagonist, and in combination with estrogens, progesterones, antiestrogens, antiprogestins and/or androgens for the prevention or treatment of bone loss or hypogonadal symptoms such as hot flashes during therapy with the GnRH antagonist; 5a-reductase 2 inhibitor, 5a-reductase 1 inhibitor, dual inhibitors of 5a-reductase 1 and 5a-reductase 2, antiandrogens, and alpha-1 blockers are useful as well; growth hormone, growth hormone releasing hormone or growth hormone secretagogues, to delay puberty in growth hormone deficient children; a compound having luteinizing hormone releasing activity is useful as well.

Definitions

An optionally substituted moiety may be substituted with one or more substituents. The substituent groups which are optionally present may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups; in one embodiment, the substituent is a halogen atom or a lower alkyl or lower alkoxy group. Typically, 0-4 substituents may be present. When any of the foregoing substituents represents or contains an alkyl substituent group, this may be linear or branched and may contain up to 12 carbon atoms, in one embodiment, up to 6 carbon atoms, in another embodiment, up to 4 carbon atoms.

As used herein, the term “alkyl” includes both branched and straight-chain saturated aliphatic hydrocarbon groups containing from 1 to 12 carbon atoms, or in some instances, from 1 to 6 carbon atoms, e.g. methyl (Me), ethyl (Et), propyl (Pr), isopropyl (i-Pr), isobutyl (i-Bu), secbutyl (s-Bu), tertbutyl (t-Bu), isopentyl, and isohexyl. The term “alkyl” further includes both unsubstituted and mono-, di- and tri-substituted hydrocarbon groups. In one embodiment, the alkyl group is substituted with a halogen.

The term “alkenyl” refers to an unsaturated or partially unsaturated aliphatic hydrocarbon group having 2 to 8 carbon atoms, for example ethenyl, 1-propenyl, and 2-butenyl. The term “alkenyl” further includes both unsubstituted and mono-, di- and tri-substituted hydrocarbon groups. In one embodiment, the alkenyl group is substituted with a halogen.

The term “cycloalkyl” includes cyclized alkyl chains having the specified number of carbon atoms, e.g., 3 to 12 or 3 to 8 carbons such as cyclopropyl, cyclobutyl, cyclopentyl, and cyclohexyl.

The term “cycloalkenyl” includes cyclized alkyl chains containing an alkenyl group having the specified number of carbon atoms, e.g., 5 to 12 carbons such as cyclopentenyl or cyclohexenyl.

The term “heterocycloalkyl” includes a 3 to 15 membered saturated or partially saturated cyclic moiety having one or more (e.g., up to three) heteroatoms selected from oxygen, nitrogen and sulfur and which may be optionally substituted as defined herein on any carbon or nitrogen atom available. Any heterocycloalkyl ring may be optionally substituted as defined herein on any carbon or nitrogen atom available. Any substituent group on A may be further substituted as defined herein.

The term “halogen” includes fluorine, chlorine, iodine, and bromine.

The term “aryl” means an aromatic carbocyclic moiety of up to 20 carbon atoms, e.g., of from 6 to 20 or 6 to 14 carbon atoms, which may be optionally substituted, and which may be a single ring (monocyclic) or multiple rings (bicyclic, up to three rings) fused together or linked covalently. Any suitable ring position of the aryl moiety may be covalently linked to the defined chemical structure. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, and acenaphthylenyl. Examples of optional substituents on an “aryl” group include those substituents identified above at paragraphs [0023] and [0025].

The term “phenyl”, as used herein, whether used alone or as part of another group, refers to a substituted or unsubstituted phenyl group. Examples of optional substituents on a “phenyl” group include those substituents identified above at paragraphs [0023] and [0025].

The term “arylalkyl” means aryl, as herein before defined, suitably substituted on any open ring position with an alkyl moiety wherein the alkyl chain is either a (C₁-C₆) straight or (C₂-C₇) branched-chain saturated hydrocarbon moiety. Examples of arylalkyl moieties include, but are not limited to, chemical groups such as benzyl, 1-phenylethyl, 2-phenylethyl, diphenylmethyl, 3-phenylpropyl, 2-phenylpropyl, fluorenylmethyl, and their homologs and isomers. Examples of optional substituents on an “arylalkyl” group include those substituents identified above at paragraphs [0023] and [0025].

The term “heteroarylalkyl” means aryl, as herein before defined, suitably substituted on any open ring position with an alkyl moiety wherein the alkyl chain is either a (C₁-C₆) straight or (C₂-C₇) branched-chain saturated hydrocarbon moiety. Examples of optional substituents on an “heteroarylalkyl” group include those substituents identified above at paragraphs [0023] and [0025].

The term “heteroaryl” means a cyclic moiety of up to 20 ring atoms, e.g., of 5-20, 5-10 or 5-8 ring atoms, which may be a single ring (monocyclic) or multiple rings (bicyclic, up to three rings) fused together or linked covalently and incorporating one or more “heteroatoms” such as nitrogen, oxygen and sulfur, e.g., having one to four heteroatoms in a ring, and having at least one aromatic ring. Any suitable ring position of the heteroaryl moiety may be covalently linked to the defined chemical structure. Examples of heteroaryl moieties include, but are not limited to, chemical groups such as pyridinyl, pyrazinyl, pyrimidinyl, furan, thiophene, pyrrole, oxazole, isoxazole, thiazole, triazole, oxadiazole, thiadiazole, quinoline, isoquinoline, quinoxaline, pyridopyrazine, benzimidazole, benzoxazole, and benzothiazole. Examples of optional substituents on a “heteroaryl” group include those substituents identified above at paragraphs [0023] and [0025].

The term “heterocycle” means a cyclic moiety of up to 20 carbon atoms, which may be a single ring (monocyclic) or multiple rings (bicyclic, up to three rings) fused together or linked covalently and incorporating one or more “heteroatoms” such as nitrogen, oxygen and sulfur. Any suitable ring position of the heterocycle moiety may be covalently linked to the defined chemical structure. Examples of heterocycle moieties include, but are not limited to, chemical groups such as pyrrolidine, tetrahydrofuran, sulfolane, piperazine, piperidine, homopiperazine, hexamethylenediamine, 1,2,3,4-tetrahydroquinoline, and 1,2,3,4-tetrahydroisoquinoline.

The compounds of the present invention can be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalene sulfonic, benzenesulfonic, toluenesulfonic, camphorsulfonic, and similarly known acceptable acids when a compound of this invention contains a basic moiety. Salts may also be formed from organic and inorganic bases, including alkali metal salts, for example, sodium, lithium, or potassium, when a compound of this invention contains a carboxylate or phenolic moiety, or similar moiety capable of forming base addition salts.

As used in accordance with this invention, the term “providing,” with respect to providing a compound or substance covered by this invention, means either directly administering such a compound or substance, or administering a prodrug, derivative, or analog which will form the effective amount of the compound or substance within the body. This invention also covers providing the compounds of this invention to treat the disease states disclosed herein that the compounds are useful for treating.

The reagents used in the preparation of the compounds of this invention can be either commercially obtained or can be prepared by standard procedures described in the literature.

The carbon number as used in the definitions herein refers to carbon backbone and carbon branching, but does not include carbon atoms of the substituents, such as alkoxy substitutions.

The term “tautomer” as used herein refers to compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992).

Tautomers often exist in equilibrium with each other. As these tautomers interconvert under environmental and physiological conditions, they provide the same useful biological effects. The present invention encompasses mixtures of such tautomers.

For the sake of simplicity, connection points (“-”) are not depicted. When an atom or compound is described to define a variable, it is understood that it is intended to replace the variable in a manner to satisfy the valency of the atom or compound. For example, when L is C(R₃)═C(R₃), both carbon atoms form a part of the ring in order to satisfy their respective valences.

The term “patient”, as used herein, refers to a mammal, in some embodiments, a human.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The compounds of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, in one embodiment, less than about 50%, in another embodiment, less than about 75%, and in yet another embodiment, less than about 90%.

The terms “effective amount”, “therapeutically effective amount” and “effective dosage” as used herein, refer to the amount of a compound that, when administered to a patient, is effective to at least partially ameliorate (and, in preferred embodiments, cure) a condition from which the patient is suspected to suffer.

The term “carrier”, as used herein, encompasses carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

Methods of Making

As shown in the following schemes, preparation of compounds of the present embodiment include the following transformations using conventional synthetic methods and, if required, standard separation and isolation techniques.

It is understood that the following schemes are to show generally how to make compounds of Formula I. As schematics, they are necessarily limited for ease of presentation, and thus not all contemplated variables are depicted. The intermediate 4 can be prepared in two ways (Schemes 1 and 2). In scheme 1 2,6-difluoronitrobenzene 1 was treated with a slight excess of sodium azide for 2 hours then the reaction mixture was treated with a 50% excess of piperazine, 2-substituted piperazine or 2,6-disubstituted piperazine in unprotected form or protected at the more hindered nitrogen as a Boc or Cbz function. Intermediate 2 was obtained in yields ranging from 50-90%. The nitro and azide functions were reduced under standard catalytic conditions (H₂, Pt/C, MeOH) and the product phenylenediamine was treated with a substituted benzaldehyde and Pd/C to promote oxidation. The product benzimidazole was deprotected if necessary (H₂, Pd/C if PG=Cbz; TFA-DCM if PG=Boc) and the product, in most cases, could be crystallized from acetonitrile.

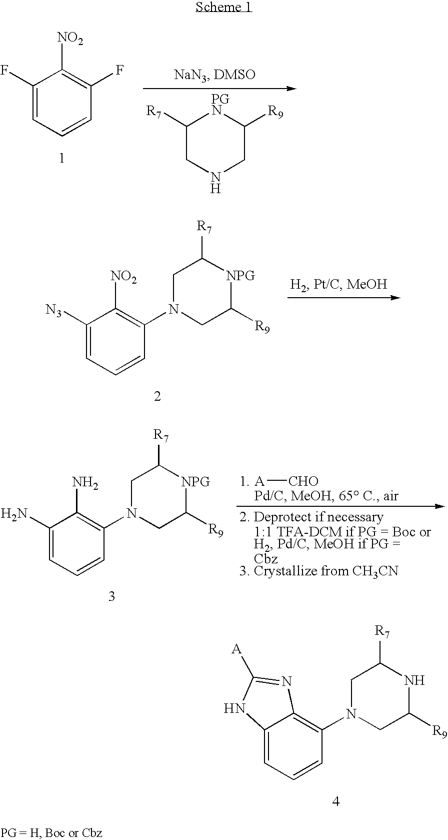

Scheme 2 indicates that the phenylenediamine intermediate 3 can be condensed with an acid and the product amide can be reacted with weak acid to cyclize and provide the intermediate 4 after deprotection.

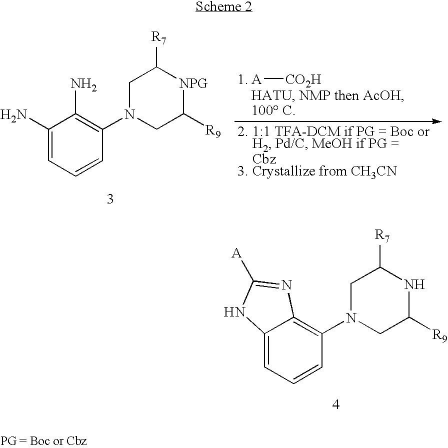

Scheme 3 shows N-alkylation occurring through nucleophilic substitution of an alkyl halide to provide the target products (I).

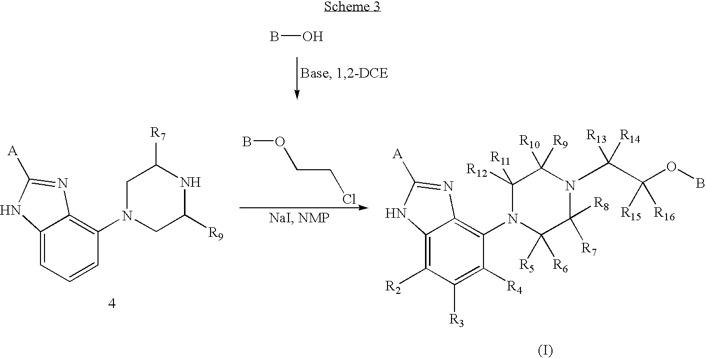

Scheme 4 indicates intermediates (6 and 7) were prepared via nucleophilic aromatic substitution of 1 with sodium azide and the sodium salt of hydroxyethylpiperazine to provide 5. The nitro and the azide groups of this intermediate were reduced, the resulting phenylenediamine was treated with thiocarbonyldiimidazole (thioCDI) followed by TFA deprotection to provide 6 or treated with hot TFA to provide 7.

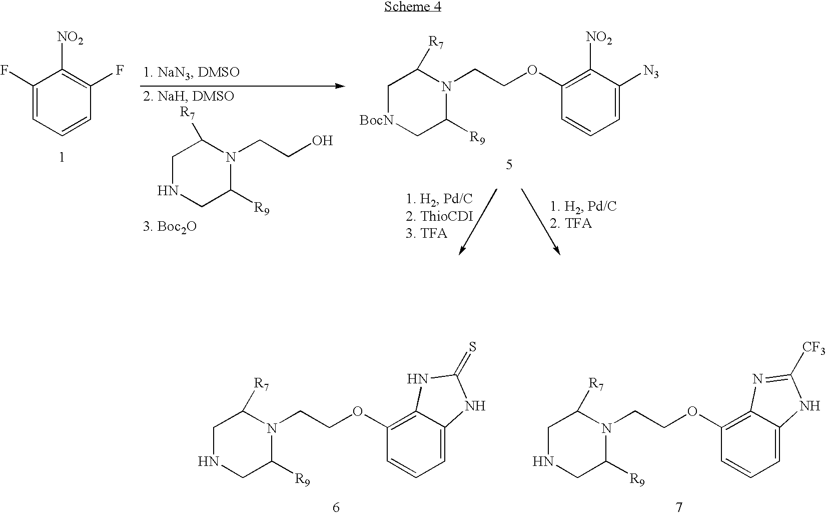

Scheme 5 indicates intermediates (6 and 7) can be converted to compounds encompassed by Formula I via treatment with 2-azido-6-fluoronitrobenzene followed by reduction of the nitroazide. The phenylenediamine can be converted as shown above.

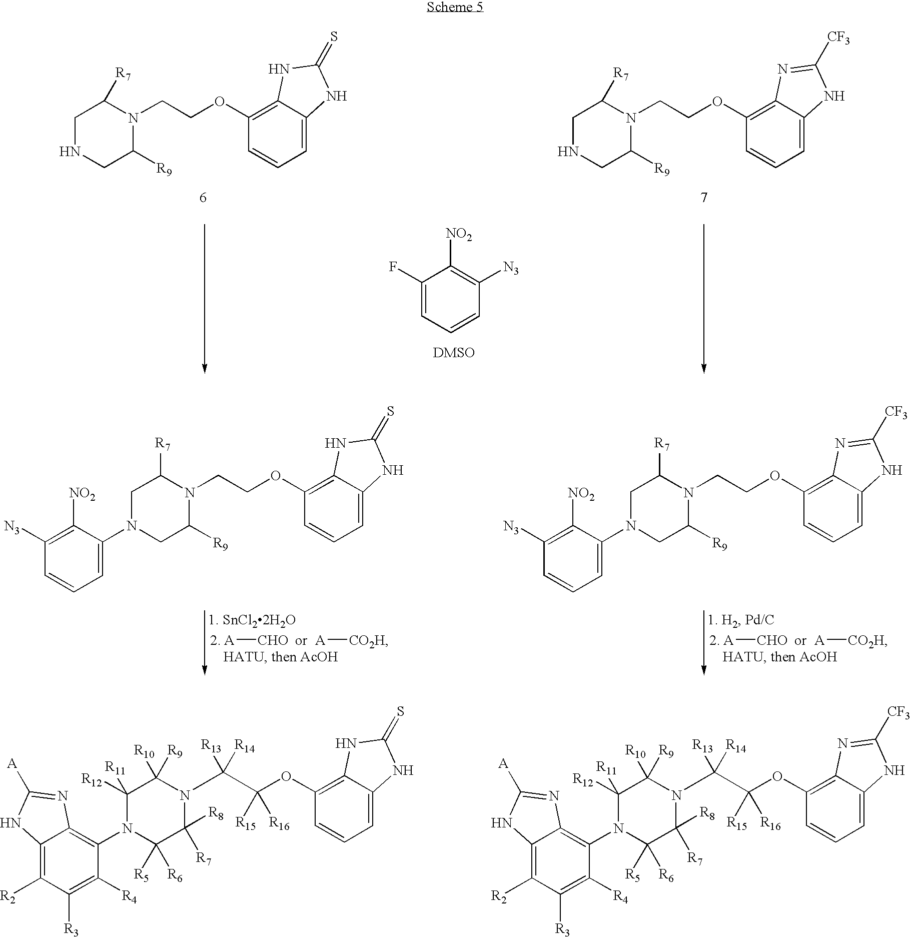

Scheme 6 shows nucleophilic aromatic substitution works with the hydroxyethylpiperazine as well. The intermediate is reduced, converted to the benzimidazole as above and the alcohol is substituted for an aryloxy group to provide (I).

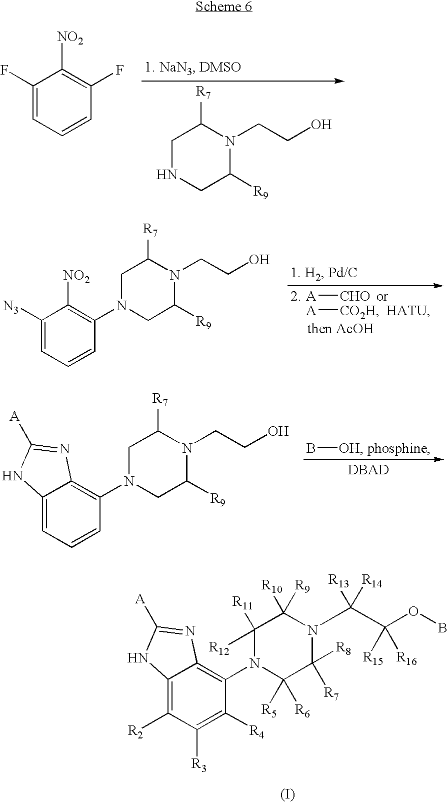

The present compounds are further described in the following examples. HPLC and LC/MS methods for the following examples and intermediates include:

Method A: Column; Xterra MS C18, 5 u, 50×2.1 mm. Mobile phase: 90/10-5/95 water (0.1% formic acid)/acetonitrile (0.1% formic acid), 2 min, hold 1.5 min, 0.8 mL/min., 210-400 nm.

Method B: LC/MS: YMC CombiScreen ProC18 50×4.6 mm I.D. column, S-5 μm, 12 nm. Flow rate 1.0 mL/min. Gradient: 10/90 Acetonitrile/Water (0.1% TFA in both solvents) to 100% acetonitrile over 10 minutes. Hold 100% acetonitrile for 3 mins then back to 10/90 over 2 mins. MS detection using a ThermoFinnigan AQA mass spectrometer in ESI positive mode.

Method C: Column; Xterra RP18, 3.5 u, 150×4.6 mm. Mobile phase: 85/15-5/95 Ammonium formate buffer (Ph=3.5)/ACN+MeOH (1:1) for 10 min, hold 4 min, 1.2 mL/min., 210-370 nm.

Method D: Column; Xterra RP18, 3.5 u, 150×4.6 mm. Mobile phase: 85/15-5/95 Phosphate buffer (Ph=2.1)/ACN+MeOH (1:1) for 10 min, hold 4 min, 1.2 mL/min., 210-370 nm.

Method E: Method E-YMC CombiPrep ProC18 50×20 mm I.D. column, S-5 μm, 12 nm. Flow rate 20 mL/min. Gradient: 10/90 Acetonitrile/Water (0.1% TFA in both solvents) to 100% acetonitrile over 10 minutes then hold for three minutes at 100% acetonitrile and ramp back to 10/90 acetonitrile/water over two minutes.

### EXAMPLES

### Example 1

### 4-[2-(3-Azido-2-nitro-phenoxy)-ethyl]-piperazine-1-carboxylic acid tert-butyl ester

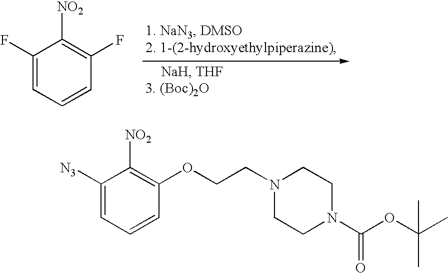

A solution of 2,6-difluoronitrobenzene (10 g, 63 mMol) in DMSO (70 mL) was treated with sodium azide (4.5 g, 69 mMol) and stirred for 2 h. The reaction mixture was diluted with ethyl acetate (500 mL), washed (water, 3×500 mL; brine, 100 mL), dried (MgSO₄) and evaporated under reduced pressure to leave the product as a tan oil that crystallized on standing (11 g, 96%). A solution of 1-(2-hydroxyethyl)piperazine (5.7 g, 43 mMol) in THF (40 mL) under nitrogen was cooled in an ice bath and treated with sodium hydride (60% mineral oil dispersion, 1.7 g, 43 mMol) portion wise over 15 mins. The mixture stirred an additional hour and was then cooled to −78° C. A solution of 2-azido-6-fluoronitrobenzene (6.0 g, 33 mMol) in THF (40 mL) was added to the reaction mixture drop wise over 15 mins. The mixture stirred an additional 2 h while warming to 20° C. then diluted with 1N HCl (50 mL) and water (500 mL). The mixture was washed with ethyl acetate (2×500 mL) and the combined organic layers were further extracted with 1 N HCl (2×100 mL) and water (200 mL). All aqueous layers were combined, neutralized (solid sodium carbonate) and extracted with chloroform (3×200 mL). The extract was dried (MgSO₄) and evaporated to leave the product as an oil (7.1 g, 24 mMol, 74%). It was dissolved in DCM (100 mL), treated with di-(t-butyl)dicarbonate and stirred for 30 mins. Aminomethylpolystyrene (1% DVB, 3.2 mMol/g, 7.5 g, 24 mMol) was added and stirring continued for 1 h. The resin was filtered, washed (DCM, 2×25 mL) and the combined organic washes were evaporated under reduced pressure to leave the product as a brown gum (9.4 g, 100%). LC/MS (method A); Rt=1.11 mins, purity=85%, [M+H]+=393.

### 4-[2-(2-Thioxo-2,3-dihydro-1H-benzoimidazol-4-yloxy)-ethyl]-piperazine-1-carboxylic acid tert-butyl ester

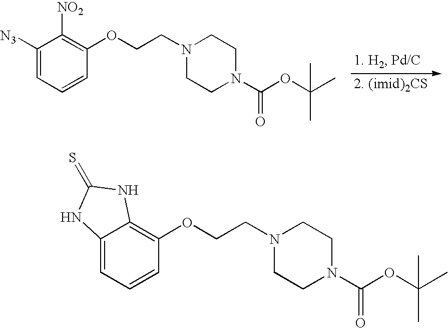

The nitroazide compound from the above procedure (9.4 g, 24 mMol) was hydrogenated (1 atmosphere H₂ pressure) over 10% palladium on carbon (1.5 g) in methanol (100 mL) for 18 hrs. The catalyst filtered with the aid of diatomaceous earth and washed with methanol (2×25 mL). The combined filtrates were evaporated to leave a brown gum (8.0 g, 99%). The product was dissolved in THF (100 mL) under nitrogen and anhydrous conditions and treated with thiocarbonyldiimidazole (7.6 g, 43 mMol). The reaction mixture stirred for 18 h, water (15 mL) was added and stirring continued for 18 h. The THF was evaporated, the residue was treated with ethyl acetate (300 mL) and washed with water (3×100 mL) and brine (100 mL). The organic phase was dried (MgSO₄), evaporated and the residue was chromatographed on silica gel eluted with a gradient of hexane—ethyl acetate (50%-60%-70%-80%-100%). The product was a tan powder (4.3 g, 47%). LC/MS (method A); Rt=0.64 mins, purity=95%, [M+H]+=379, [M+H]−=377.

### 4-{2-[4-(2,3-Diamino-phenyl)-piperazin-1-yl]-ethoxy}-1,3-dihydro-benzoimidazole-2-thione

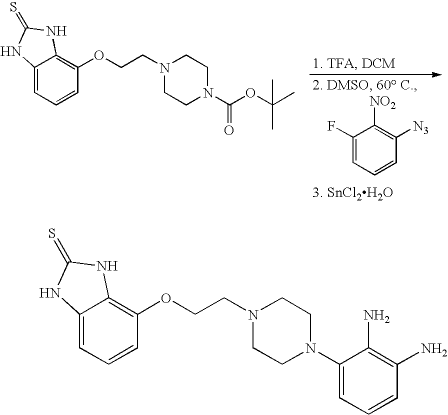

A suspension of the Boc protected piperazine from above (4.3 g, 11 mMol) in DCM (80 mL) was treated with polystyrene supported dimethylsilane (1% DVB, 1.7 mMol/g, 13 g, 22 mMol, NovaBiochem) then TFA (20 mL) and stirred for an hour. The resin was filtered and washed (DCM, 2×25 mL) and the combined filtrates were evaporated. The residue was treated twice with toluene and evaporated to remove excess TFA. The crude product was dissolved in water (100 mL), treated with sodium carbonate (5.0 g) and the solution was saturated with sodium chloride. The product was extracted with n-butanol (2×50 mL) and the combined extracts were dried (Na₂SO₄) and evaporated under reduced pressure to leave the product as a hygroscopic light tan powder (2.1 g, 69%). A solution of this product (1.8 g, 6.5 mMol) in DMSO (18 mL) was treated with 2-azido-6-fluoronitrobenzene (1.8 g, 9.7 mMol) and stirred at 60° C. for 24 h. The reaction mixture was cooled to room temperature, diluted with ethyl acetate (300 mL) and washed with 1 M sodium carbonate solution (100 mL). The aqueous layer was washed with ethyl acetate (50 mL) and the combined organic phases were washed (water, 3×100 mL and brine, 100 mL), dried (MgSO₄) and evaporated. The residue was chromatographed on silica gel eluted with a gradient (75% ethyl acetate in hexanes to 100% ethyl acetate) to leave the product as a yellow foamy solid (1.9 g, 64%). The nitroazide product (1.8 g, 4.1 mMol) was dissolved in NMP (40 mL) and treated with tin(II) chloride dihydrate (9.2 g, 41 mMol). The mixture stirred for 5 mins at 20° C. then 1.5 h at 100° C. After cooling to room temperature the mixture was diluted with 1N HCl (30 mL) and filtered through diatomaceous earth. The filtrate was neutralized with solid sodium carbonate and ethyl acetate (200 mL) was added. After stirring 15 mins. The mixture was filtered through diatomaceous earth, the filtrate layers were separated and the organic layer was washed with water (5×100 mL), brine (100 mL), dried (Na₂SO₄) and evaporated under reduced pressure to leave the product as a light yellow powder (0.68 g, 43%). LC/MS (method A); Rt=0.22 mins, purity=92.5%, [M+H]+=385.

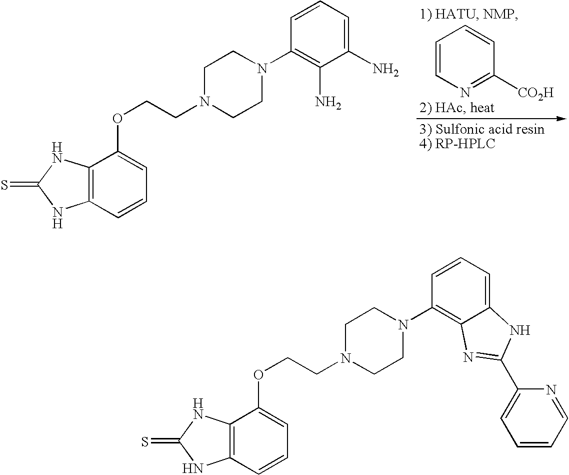

To an 8 mL screw cap vial was added N-Methyl pyrrolidinone (4 ml/vial) and 279 microliters of a 0.2 M solution of O-(7-Azabenzotriazol-1-yl)-N,N,N′,N′-tetramethyl-uronium hexafluorophosphate (HATU) in NMP. To this was added 17.8 mg (0.046 mMol) of 4-{2-[4-(2,3-Diamono-phenyl)-piperazin-1-yl]-ethoxy}-1,3-dihydro-benzimidazole-2-thione followed by picolinic acid (6.8 mg, 0.056 mmol) and the vial capped tightly. The vial was shaken overnight on an orbital shaker and then treated with 0.5 mL of glacial acetic acid. The vial was then recapped and shaken at 110 degrees for two hours. The vial was then removed from the heat and shaken overnight at room temperature. To the vial was then added sulfonic acid resin (Argonaut, 132 mg, 1.4 mmol/g) and the vial shaken for six hours.

The reaction mixture was then filtered using polypropylene filter tubes (15 mL) and the resin washed with MeOH (3×3 mL) followed by dichloromethane (2×3 mL). A 1.5 mL portion of MeOH:Triethylamine (9:1) was added to the resin and it was capped tightly. After loosely shaking for three minutes the reaction was filtered into a 13×100 mm test tube and the solvent removed by a Savant speedvac overnight. The crude product was then purified by automated RP-HPLC (Method E) and the fractions evaporated in 8 mL scintillation vial. The product was characterized by LC/MS (Method B): Rt=5.07 min [M+H] 472, purity 100% @ 220 nm, 100% @ 254 nm to yield 2.3 mg of 4-{2-[4-(2-Pyridin-2-yl-1H-benzoimidazol-4-yl)-piperazin-1-yl]-ethoxy}-1,3-dihydro-benzoimidazole-2-thione.

### 3-(2-piperazine-1-yl-ethoxy)-benzene-1,2,-diamine

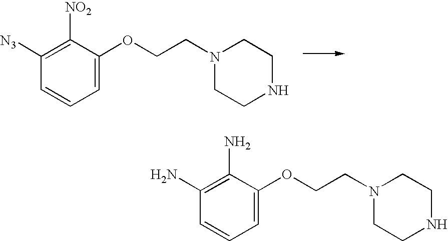

In a round bottom flask under nitrogen was placed 11.88 g (40.6 mmol) of 1-[2-(3-Azido-2-nitro-phenoxy)-ethyl]-piperazine and 400 mL of methanol. 10% Palladium on carbon (4.3 g, 4.06 mmol) was carefully added and a hydrogen filled balloon attached. The flask was placed under vacuum and allowed to refill from the balloon. The solution was stirred under a hydrogen atmosphere overnight. Upon arrival the balloon was removed and a nitrogen atmosphere established. The solution was filtered thru Celite and the residue washed well with methanol. Concentration of the methanol solution to dryness on a rotovap yielded (9.3 g, 97% yield) of 3-(2-piperazin-1-yl-ethoxy)-benzene-1,2-diamine as an orange-brown solid. 1H NMR (CDCl3): 7.27 (br s,2H), 6.64 (t, 1H,J=7.9 Hz), 6.41 (m,2H), 4.12(t,2H,J=5.7 Hz), 2.94 (m,4H), 2.80 (t,2H,J=5.7 Hz), 2.59 (m,4H). LC/MS (Method A), rt=0.25 mins, calculated mass=236, [M+H]+237.

### 4-(2-piperazin-1-yl-ethoxy)-2-trifluoromethyl-1H-benzoimidazole

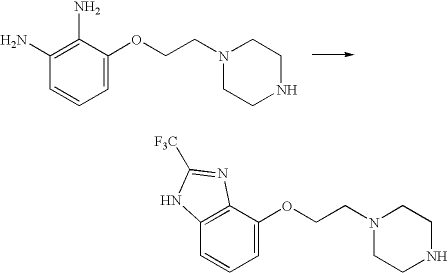

In a round bottom flask were combined 9.3 g (39.4 mmol) of 3-(2-piperazin-1-yl-ethoxy)-benzene-1,2-diamine and trifluoroacetic acid (50 mL) and the mixture stirred at seventy degrees Celsius overnight. Upon arrival the mixture was concentrated to near dryness on a rotovap and then partitioned between ethyl acetate (300 mL) and 1N sodium hydroxide (500 mL). The organic layer was discarded and the basic layer adjust to pH=5 with acetic acid. Sodium bicarbonate was then added until the pH was equal to eight. At this point the solution was saturated with sodium chloride and then extracted with chloroform (5×250 mL). The organics were dried with magnesium sulfate, filtered, and concentrated to dryness on a rotary evaporator to yield 13.6 g (109%) of 4-(2-piperazin-1-yl-ethoxy)-2-trifluoromethyl-1H-benzoimidazole as a yellow solid. 1H NMR (DMSO-d6): 7.16 (m,2H), 6.70 (d, 1H,J=7.3 Hz), 4.30 (t,2H,J=5.7 Hz), 2.86 (m,4H), 2.77 (t,2H,J=5.7 Hz), 2.54 (m,4H). LC/MS (Method A), rt=0.28 mins, calculated mass=314, [M+H]+315.

### 4-{2-[4-(3-azido-2-nitrophenyl)-piperazin-1-yl]-ethoxy}-2-trifluoromethyl-1H-benzoimidazole

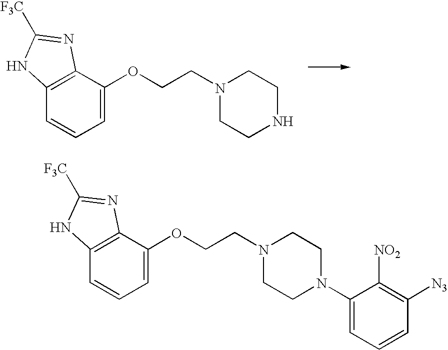

In a round bottom flask under nitrogen were combined 4-(2-piperazin-1-yl-ethoxy)-2-trifluoromethyl-1H-benzoimidazole (13.6 g, 43.5 mmol), diisopropylethylamine (22.7 mL, 130.5 mmol), and 1-Azido-3-fluoro-2-nitro-benzene (7.9 g, 43.5 mmol) in dimethyl sulfoxide (200 mL). Monitoring by LC/MS showed reaction complete after four days. The solution was diluted with ethyl acetate (500 mL) and washed with set's sodium bicarbonate solution (100 mL), water (3×250 mL), and brine (250 mL). The organic layer was dried with magnesium sulfate, filtered, and concentrated to dryness. Purification by flash column chromatography using 40-60% Ethyl acetate in dichloromethane as eluant yielded 7.5 g (36% yield) of 4-{2-[4-(3-Azido-2-nitro-phenyl)-piperazin-1-yl]-ethoxy}-2-trifluoromethyl-1H-benzoimidazole as a yellow solid. 1H NMR (CDCl3): 7.51 (br s, 1H), 7.43 (t, 1H,J=8.2 Hz), 7.29 (m,2H), 6.99 (t,2H,J=8.4 Hz), 6.84 (d, 1H,J=7.6 Hz), 4.30 (m,2H), 3.07 (br s,4H), 2.87 (m,2H), 2.71 (br s,4H). LC/MS (Method A), rt=6.6 mins., calculated mass=476, [M+H]+477, purity 92% @ 254 nm.

### 3-{4-[2-(2-Trifluoromethyl-1H-benzoimidazol-4-yloxy)-ethyl]piperazin-1-yl}-benzene-1,2-diamine

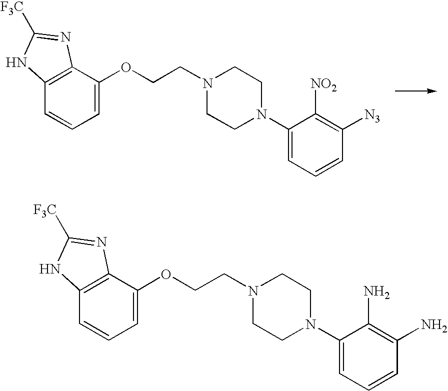

In a round bottom flask under nitrogen was placed 4.0 g (8.4 mmol) of 4-{2-[4-(3-Azido-2-nitro-phenyl)-piperazin-1-yl]-ethoxy}-2-trifluoromethyl-1H-benzoimidazole and 100 mL of methanol. 10% Palladium on carbon (0.89 g, 0.84 mmol) was carefully added and a hydrogen filled balloon attached. The flask was placed under vacuum and allowed to refill from the balloon. The solution was stirred under a hydrogen atmosphere overnight. Upon arrival the balloon was removed and a nitrogen atmosphere established. The solution was filtered thru Celite and the residue washed well with methanol. Concentration of the methanol solution to dryness on a rotovap yielded 3-{4-[2-(2-Trifluoromethyl-1H-benzoimidazol-4-yloxy)-ethyl]-piperazin-1-yl}-benzene-1,2-diamine (3.66 g, 104% yield) as a brown solid. 1H NMR (CDCl3): 7.50 (d, 1H,J=8.2 Hz), 7.25 (m, 1H), 6.81 (d, 1H,J=7.9 Hz), 6.65 (m,2H), 6.55 (dd, 1H,J=7.3, 1.55 Hz), 4.31 (m,2H), 2.95 (m, 4H), 2.89 (m,2H), 2.80 (m, 4H). LC/MS (Method A), rt=0.76 mins., calculated mass=420, [M+H]+421.

### Example 2

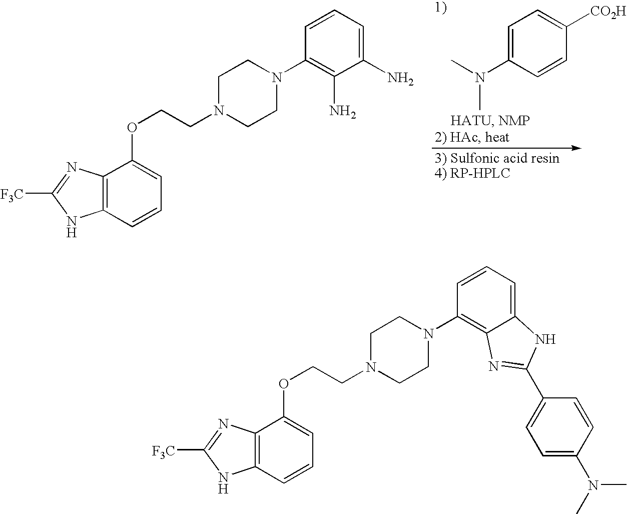

To an 8 mL screw cap vial was added N-Methyl pyrrolidinone (1 ml/vial) and 1 mL of a 0.14 M solution of O-(7-Azabenzotriazol-1-yl)-N,N,N′,N′-tetramethyluroniumhexafluoro-phosphate (HATU) in NMP. To this was added 50 mg (0.119 mMol) of 3-{4-[2-(2-Trifluoromethyl-1H-benzoimidazol-4-yloxy)-ethyl]-piperazin-1-yl}-benzene-1,2-diamine followed by 4-(Dimethylamino)benzoic acid (23.6 mg, 0.143 mmol) and the vial capped tightly. The vial was shaken overnight on an orbital shaker and upon arrival treated with 0.5 mL of glacial acetic acid and shaken at 110 degrees for two hours. The reaction was then removed from the heat and shaken overnight at room temperature. To the vial was added sulfonic acid resin (Argonaut, 340 mg, 1.4 mmol/g) and the mixture shaken for six hours.

The reaction mixture was then filtered using polypropylene filter tubes (15 mL) and the resin washed with MeOH (3×2 mL) followed by dichloromethane (2×3 mL). A PTFE stopcock was attached and 1.75 mL of 9:1 MeOH:Triethylamine was added. After loosely shaking for three minutes the reaction was filtered into a 13×100 mm test tube and the solvent removed using a Savant speedvac overnight. The crude product was then purified by automated RP-HPLC (Method E) and the fractions evaporated in 8 mL scintillation vial. The product was characterized by LC/MS (Method B): Rt=6.08 min [M+H] 551, purity 95% @ 220 and 254 nm to yield 17.5 mg of Dimethyl-[4-(4-{4-[2-(2-trifluoromethyl-1H-benzo-imidazol-4-yloxy)-ethyl]piperazin-1-yl}-1H-benzoimidazol-2-yl)-phenyl]-amine.

Table 1 indicates other compounds prepared from the same method as examples 1 and 2 using the appropriate carboxylic acid and phenylenediamine starting materials. wherein R₁ is H

TABLE 1

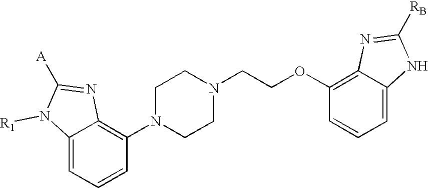

| Example | A | R_{B} | [M + H]+ |
|---|---|---|---|
| 3 | 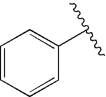 | SH | 471 |
| 4 | 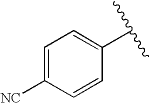 | SH | 496 |
| 5 | 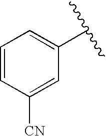 | SH | 496 |
| 6 | 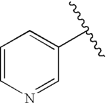 | SH | 472 |
| 7 | 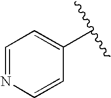 | SH | 472 |
| 8 | 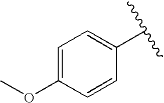 | SH | 500 |
| 9 | 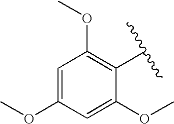 | SH | 561 |
| 10 | 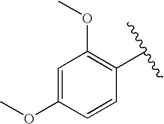 | SH | 531 |
| 11 | 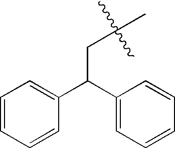 | SH | 576 |
| 12 | 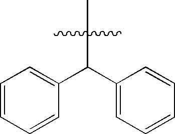 | SH | 562 |
| 13 | 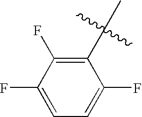 | SH | 526 |
| 14 | 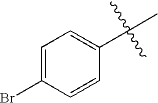 | SH | 551 |
| 15 | 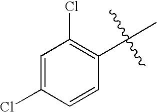 | SH | 540 |
| 16 | 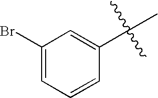 | SH | 551 |
| 17 | 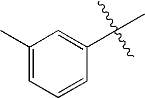 | SH | 486 |
| 18 | 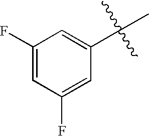 | SH | 508 |
| 19 | 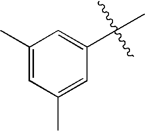 | SH | 500 |
| 20 | 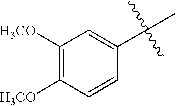 | SH | 532 |
| 21 | 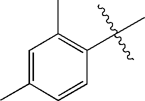 | SH | 500 |
| 22 | 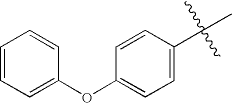 | SH | 564 |
| 23 | 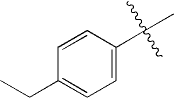 | SH | 500 |
| 24 | 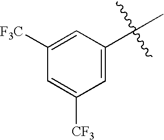 | SH | 608 |
| 25 | 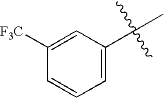 | SH | 540 |
| 26 | 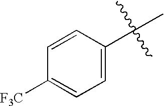 | SH | 540 |
| 27 | 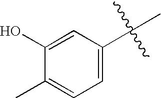 | SH | 502 |
| 28 | 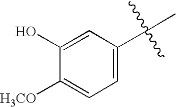 | SH | 518 |
| 29 | 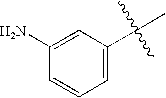 | SH | 487 |
| 30 | 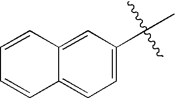 | SH | 522 |
| 31 | 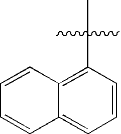 | SH | 522 |
| 32 | 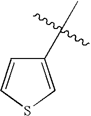 | SH | 478 |
| 33 | 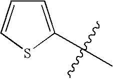 | SH | 514 |
| 34 | 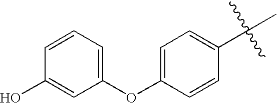 | SH | 616 |
| 35 | 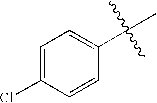 | SH | 542 |
| 36 | 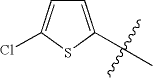 | SH | 548 |
| 37 | 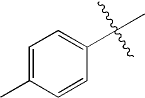 | SH | 522 |
| 38 | 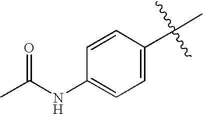 | SH | 564 |
| 39 | 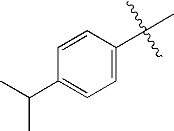 | SH | 550 |
| 40 | 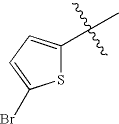 | SH | 592 |
| 41 | 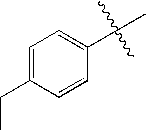 | SH | 535 |
| 42 | 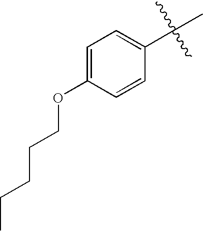 | SH | 594 |
| 43 | 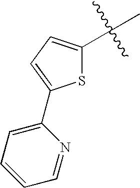 | SH | 591 |
| 44 | 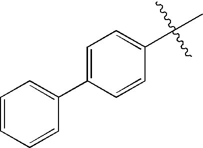 | SH | 584 |
| 45 | 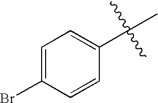 | SH | 586 |
| 46 | 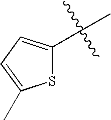 | SH | 528 |
| 47 | 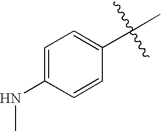 | SH | 536 |
| 48 | 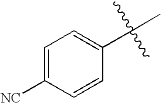 | SH | 532 |
| 49 | 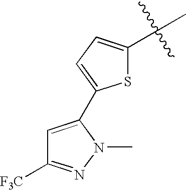 | CF₃ | 662 |
| 50 | 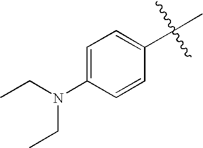 | CF₃ | 579 |
| 51 | 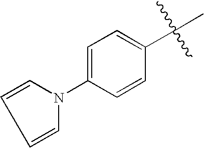 | CF₃ | 573 |
| 52 | 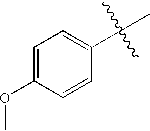 | CF₃ | 538 |
| 53 | 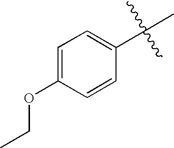 | CF₃ | 551 |
| 54 | 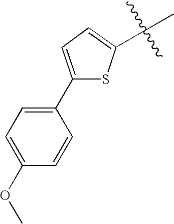 | CF₃ | 620 |
| 55 | 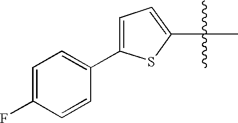 | CF₃ | 608 |
| 56 | 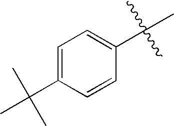 | CF₃ | 564 |
| 57 | 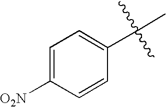 | CF₃ | 553 |
| 58 | 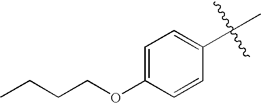 | CF₃ | 580 |
| 59 | 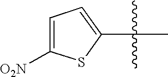 | CF₃ | 559 |
| 60 | 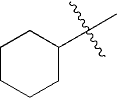 | CF₃ | 514 |
| 61 | 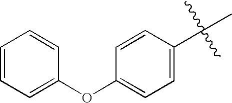 | CF₃ | 600 |
| 62 | 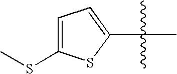 | CF₃ | 560 |
| 63 | 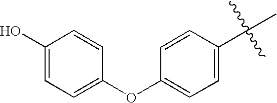 | CF₃ | 616 |
| 64 | 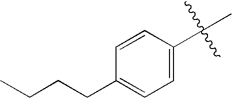 | CF₃ | 564 |
| 65 | 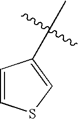 | CF₃ | 514 |
| 66 | 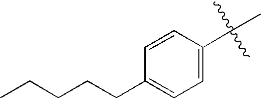 | CF₃ | 578 |
| 67 | 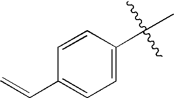 | CF₃ | 533 |
| 68 | 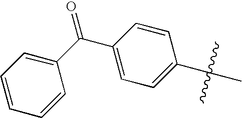 | CF₃ | 612 |
| 69 | 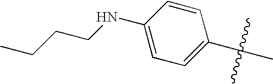 | CF₃ | 579 |
| 70 | 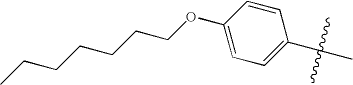 | CF₃ | 622 |
| 71 | 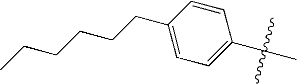 | CF₃ | 592 |
| 72 | 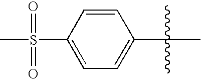 | CF₃ | 586 |
| 73 | 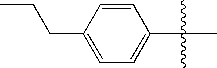 | CF₃ | 550 |
| 74 | 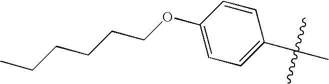 | CF₃ | 608 |
| 75 | 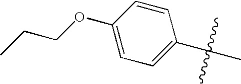 | CF₃ | 566 |
| 76 | 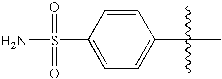 | CF₃ | 587 |
| 77 | 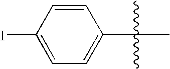 | CF₃ | 633 |
| 78 | 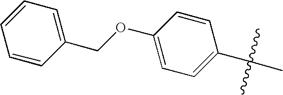 | CF₃ | 614 |
| 79 | 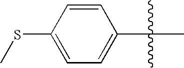 | CF₃ | 554 |
| 80 | 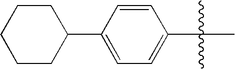 | CF₃ | 590 |
| 81 | 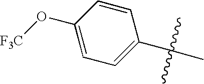 | CF₃ | 591 |
| 82 | 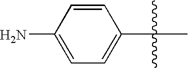 | CF₃ | 522 |
| 83 | 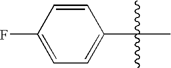 | CF₃ | 525 |
| 84 | 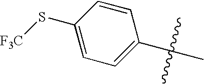 | CF₃ | 608 |
| 85 | 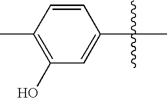 | CF₃ | 537 |
| 86 | 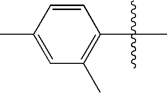 | CF₃ | 535 |
| 87 | 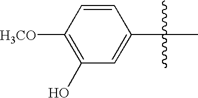 | CF₃ | 553 |
| 88 | 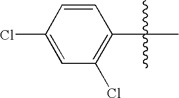 | CF₃ | 576 |
| 89 | 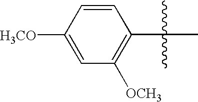 | CF₃ | 567 |
| 90 | 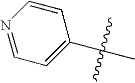 | CF₃ | 508 |
| 91 | 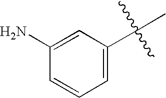 | CF₃ | 522 |
| 92 | 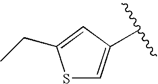 | CF₃ | 541 |
| 93 | 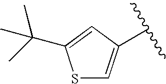 | CF₃ | 569 |
| 94 | 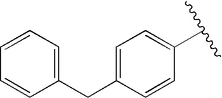 | CF₃ | 597 |
| 95 | 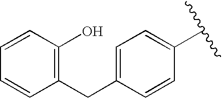 | CF₃ | 613 |
| 96 | 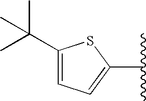 | CF₃ | 569 |
| 97 | 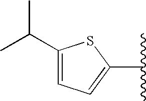 | CF₃ | 555 |
| 98 | 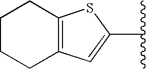 | CF₃ | 565 |

### Example 99

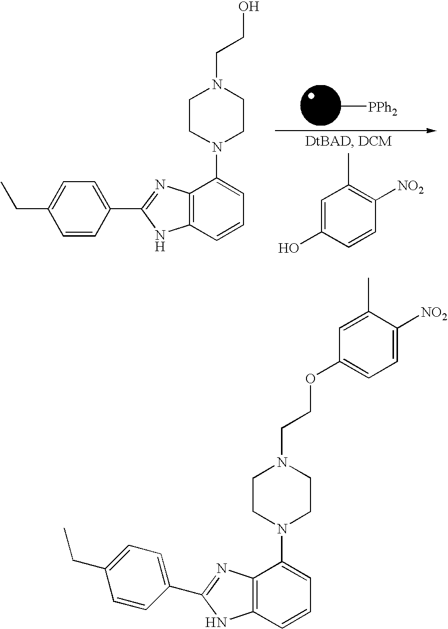

In a 20 ml vial with a Teflon cap was placed a solution of 2-{4-[2-(4-Ethyl-phenyl)-1H-benzoimidazol-4-yl]-piperazin-1-yl}-ethanol (0.103 g, 0.295 mmol) in 6 mL of dichloromethane. To this was added 3-Methyl-4-nitrophenol (0.181 g, 0.59 mmol) in 3 mL of dichloromethane followed by polymer-supported triphenylphosphine (Fluka, 0.246 g, 0.738 mmol) and the vial cooled to zero degrees in ice water. Di-t-butyl azodicarboxylate (0.153 g, 0.442 mmol) was added, the vial capped, and the reaction mixture shaken overnight. Upon completion the reaction mixture was treated with 4 mL of trifluoroacetic acid and the reaction shaken for one hour. The reaction mixture was then filtered and the resin washed with dichloromethane (3×3 mL). The combined organics were concentrated to dryness on a rotovap and then redissolved in 10 mL of ethyl acetate. The solution was washed with 5 mL of saturated sodium bicarbonate solution and the organic layer transferred to a 20 mL vial and concentrated to dryness on a Savant speedvac. The crude product was then purified by automated RP-HPLC (Method E) and the fractions evaporated in an 8 mL scintillation vial. The product was characterized by 1H NMR (DMSO-d6): δ 12.64 (s, 1H), 8.0 (m,3H), 7.34 (d,2H,J=8.3 Hz), 7.07 (d, 1H,J=2.7 Hz), 6.99 (m,2H), 6.74 (m, 1H), 6.47 (dd, 1H,J=1.35, 7.0 Hz) 4.25 (t,2H,J=5.6 Hz) 3.54 (br s, 4H), 3.24 (m,2H), 2.79 (br s,2H), 2.72 (br s,2H), 2.64 (q,2H,J=7.6 Hz), 2.53 (s,3H), 1.19 (t,3H,J=7.6 Hz) and LC/MS (Method B): Rt=5.65 min [M+H] 486, purity 99% @ 220 and 254 nm to yield 7.6 mg of 2-(4-Ethyl-phenyl)-4-{4-[2-(3-methyl-4-nitro-phenoxy)-ethyl]-piperazin-1-yl}-1H-benzoimidazole.

Table 2 indicates other compounds prepared from the same method as example 99 using the appropriate phenol and alcohol starting materials.

TABLE 2

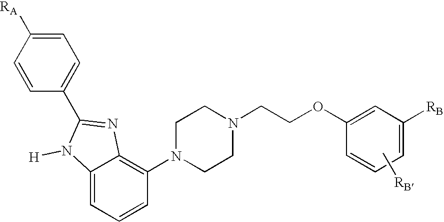

| Example | R_{A} | R_{B} | R_{B′} | [M + H]+ |
|---|---|---|---|---|
| 100 | F | Me | 4-NO₂ | 491 |
| 101 | Cl | Me | 4-NH₂ | 477 |
| 102 | F | Me | 4-NH₂ | 461 |
| 103 | CH₃ | Me | 4-NH₂ | 457 |
| 104 | H | Me | 4-NO₂ | 472 |
| 105 | H | Me | 4-NH₂ | 441 |
| 106 | H | Me | 3-NO₂ | 472 |
| 107 | H | Me | 3-NH₂ | 442 |
| 108 | H | Me | 4-OH | 443 |
| 109 | H | Me | 4-CONH₂ | 470 |
| 110 | CONH₂ | Me | 4-OH | 486 |
| 111 | H | t-Bu | 4-NH₂ | 470 |

### Example 112

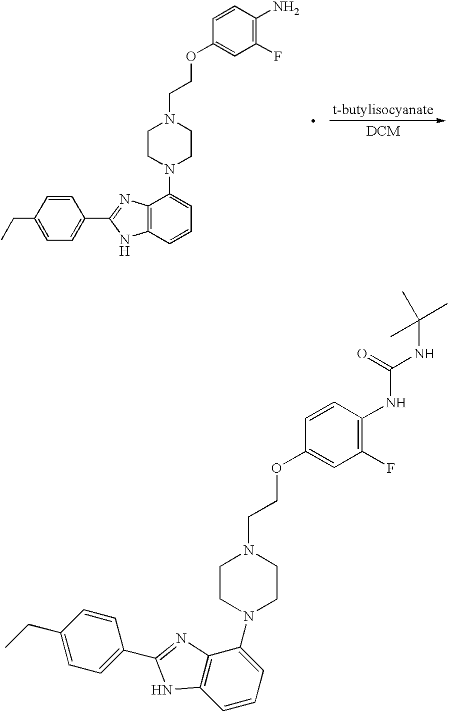

In a 8 mL scintillation vial was placed 4-(2-{4-[2-(4-Ethyl-phenyl)-1H-benzoimidazol-4-yl]piperazin-1-yl}-ethoxy)-2-fluoro-phenylamine (0.021 g, 0.047 mMol) and 5 mL of dichloromethane. To the solution was added t-butyl isocyanate (0.021 mL, 0.188 mMol) and the reaction was shaken overnight. After 24 h the reaction was incomplete. To the vial was added N-methyl pyrrolidinone (1.0 mL) and additional t-butyl isocyanate (0.10 mL). The vial was sealed and heated to 40° C. for forty-eight hours. Upon completion the reaction mixture was diluted with ethyl acetate and washed with water (2×1 mL) and then with brine (2 mL). The organic layer was then concentrated to dryness on a Savant speedvac. The crude product was then purified by automated RP-HPLC (Method E) and the fractions evaporated in an 8 mL scintillation vial. The product was characterized by LC/MS (Method B): Rt=5.11 min [M+H] 559, purity 98% @ 220 and 254 nm to yield 7.4 mg of 1-tert-Butyl-3-[4-(2-{4-[2-(4-ethyl-phenyl)-1H-benzoimidazol-4-yl]-piperazin-1-yl}-ethoxy)-2-fluoro-phenyl]-urea.

Table 3 indicates other compounds prepared from the same method as example 112 using the appropriate aniline starting material.

TABLE 3

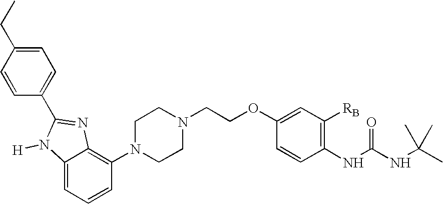

| Example | R_{B} | [M + H]+ |
|---|---|---|
| 113 | Cl | 576 |
| 114 | Me | 556 |

### Example 115

### 1-Azepan-1-yl-3-[4-(2-{4-[2-(4-tert-butyl-phenyl)-1H-benzoimidazol-4-yl]-piperazin-1-yl}-ethoxy)-phenyl]-urea

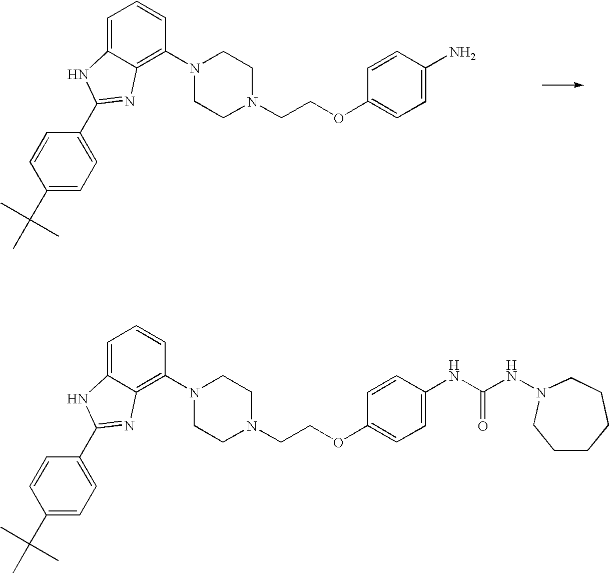

Procedure: To a 4-dram vial at room temperature was added 4-(2-{4-[2-(4-tert-butyl-phenyl)-1H-benzoimidazol-4-yl]-piperazin-1-yl}-ethoxy)-phenylamine (30 mg, 0.064 mMol) and pyridine (5.3 mg, 0.067 mMol) in THF (1 ml). To the resulting yellow solution was added 4-nitrophenyl chloroformate (11 mg, 0.070 mMol) and the vial was capped and stirred 1 hr. The solvent was removed by nitrogen stream and the off-white paste was dissolved in DMSO (0.5 ml). To the solution was added 1-(amino)homopiperidine (11.6 mg, 0.073 mMol) and the mixture stirred for 90 mins. Water (0.1 mL) was added and the product was purified by reverse phased HPLC to afford the mono-trifluoroacetic acid salt as a yellow powder (9.0 mg, 24% yield). Mass. Spec. (positive ESI) m/z 610 [M+H]+; Mass. Spec. (negative ESI) m/z 608 [M−H]−.

### Example 116

### [4-(2-{4-[2-(4-Ethyl-phenyl)-1H-benzoimidazol-4-yl]-piperazin-1-yl}-ethoxy)-phenyl]-carbamic acid allyl ester

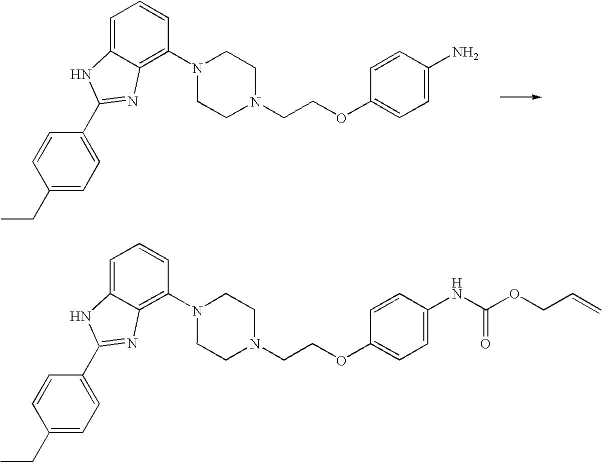

Procedure: To a 4-dram vial at room temperature was added 4-(2-{4-[2-(4-tert-Butyl-phenyl)-1H-benzoimidazol-4-yl]-piperazin-1-yl}-ethoxy)-phenylamine (25 mg, 0.056 mMol), in CH₂Cl₂ (1 ml). To the resulting yellow solution was added allyl chloroformate (6.8 mg, 0.058 mMol) and di-isopropylethyl amine (8.5 mg, 0.067 mMol) and the vial was capped and stirred 1 hr. The solvent was removed by nitrogen stream and the resulting crude product was purified by reversed phase HPLC. The mono-trifluoroacetic acid salt was isolated as a light yellow powder (7.0 mg, 10% yield). Mass. Spec. (positive ESI) m/z 526 [M+H]+; Mass. Spec. (negative ESI) m/z 524 [M−H]−.

### Example 117

### 4-ethyl-N-[4-(2-{4-[2-(4-ethylphenyl)-1H-benzimidazol-4-yl]piperazin-1-yl}ethoxy)phenyl]benzamide

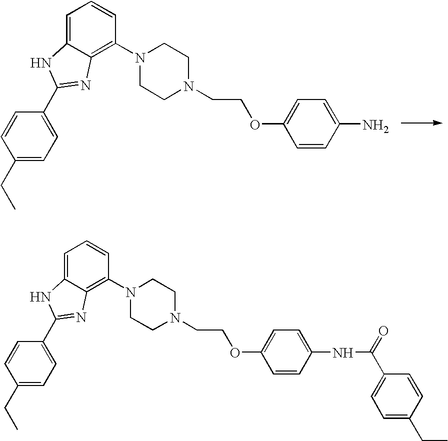

Procedure: A solution of 4-(2-{4-[2-(4-ethylphenyl)-1H-benzimidazol-4-yl]piperazin-1-yl}ethoxy)aniline (34 mg, 0.077 mMol) in CH₂Cl₂ (5 ml) at 0° C. under a nitrogen atmosphere was treated with 4-ethylbenzoyl chloride (12.0 mg, 0.073 mMol) and diisopropylethylamine (12 mg, 0.092 mMol). The resulting slightly cloudy yellow solution was stirred for 90 mins., quenched with water (50 μl) and concentrated in vacuo to brown syrup. Purification by reverse phased HPLC (method E) afforded the mono-trifluoroacetic acid salt as a white powder (23.6 mg, 54% yield). Mass. Spec. (ESI) m/z 574 ([M+H]+; Mass. Spec. (ESI) m/z 572 ([M−H]−.

### Example 118

### N-[4-(2-{4-[2-(4-ethylphenyl)-1H-benzimidazol-4-yl]piperazin-1-yl}ethoxy)phenyl]-2-propane sulfonamide

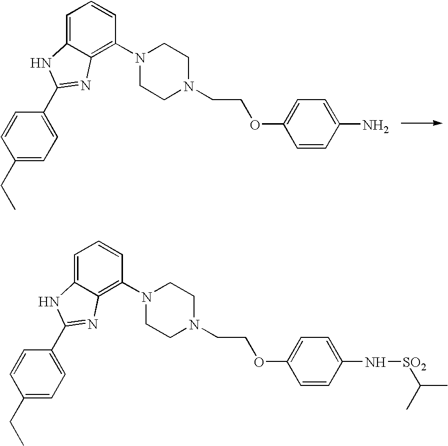

A solution of 4-(2-{4-[2-(4-ethylphenyl)-1H-benzimidazol-4-yl]piperazin-1-yl}ethoxy)aniline (25 mg, 0.056 mMol) in CH₂Cl₂ (2 ml) at room temperature was treated with 2-propanesulfonyl chloride (7.9 mg, 0.056 mMol) and diisopropylethylamine (8.8 mg, 0.068 mmol). The resulting slightly cloudy yellow solution was stirred for 90 mins., quenched with water (50 μl) and concentrated in vacuo to a brown gum. Purification by reversed phased HPLC afforded the mono-trifluoroacetic acid salt as a yellow powder (5.2 mg, 16% yield). Mass. Spec. (ESI) m/z 562 ([M+H]+; Mass. Spec. (ESI) m/z 560 ([M−H]−.

TABLE 4

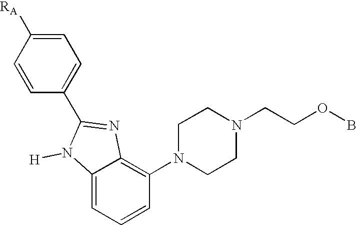

| Example | R_{A} | B | [M + H]+ |
|---|---|---|---|
| 119 | Et | 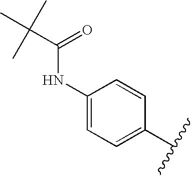 | 525 |
| 120 | Et | 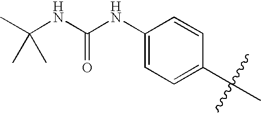 | 541 |
| 121 | Et | 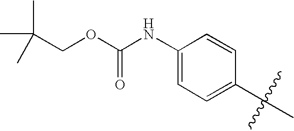 | 556 |
| 122 | t-Bu | 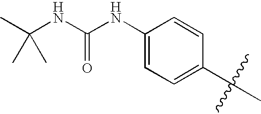 | 569 |
| 123 | Et | 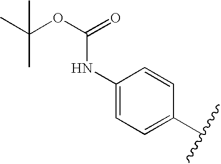 | 542 |
| 124 | Et | 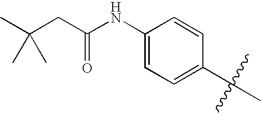 | 540 |
| 125 | Et | 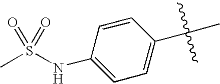 | 520 |
| 126 | Et | 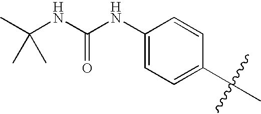 | 541 |
| 127 | Et | 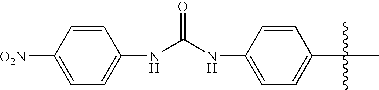 | 606 |
| 128 | Et | 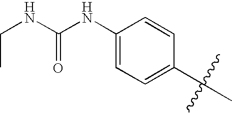 | 513 |
| 129 | Et | 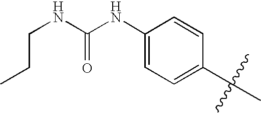 | 527 |
| 130 | Et | 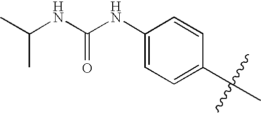 | 527 |
| 131 | Et | 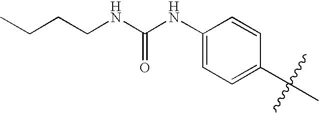 | 541 |
| 132 | Et | 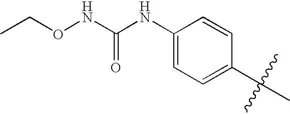 | 557 |
| 133 | Et | 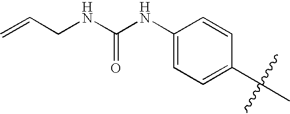 | 525 |
| 134 | Et | 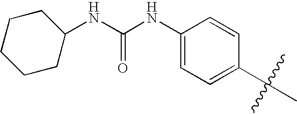 | 567 |
| 135 | Et | 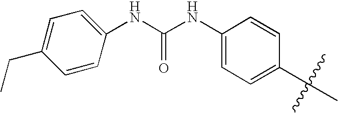 | 589 |
| 136 | Et | 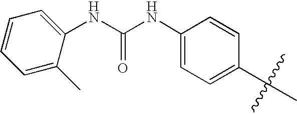 | 575 |
| 137 | Et | 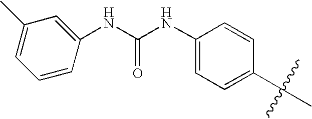 | 575 |
| 138 | Et | 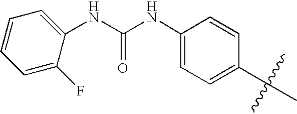 | 579 |
| 139 | Et | 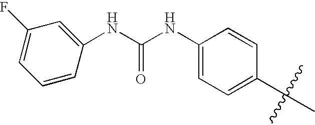 | 579 |
| 140 | Et | 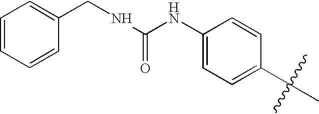 | 575 |
| 141 | Et | 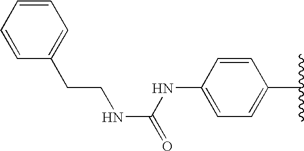 | 589 |
| 142 | Et | 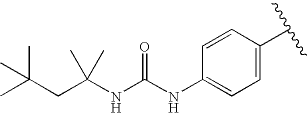 | 597 |
| 143 | Et | 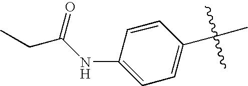 | 498 |
| 144 | Et | 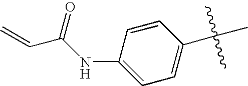 | 496 |
| 145 | Et | 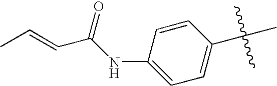 | 510 |
| 146 | Et | 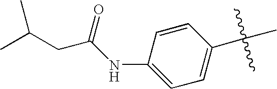 | 526 |
| 147 | Et | 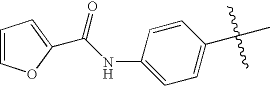 | 536 |
| 148 | Et | 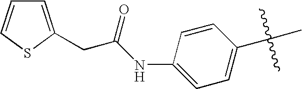 | 566 |
| 149 | Et | 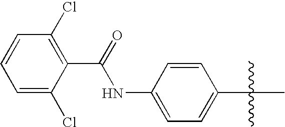 | 615 |
| 150 | Et | 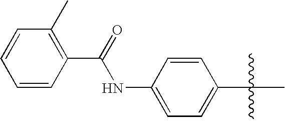 | 560 |
| 151 | Et | 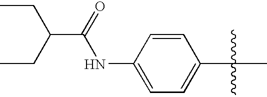 | 540 |
| 152 | Et | 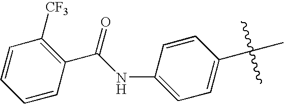 | 614 |
| 153 | Et | 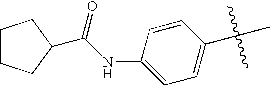 | 538 |
| 154 | Et | 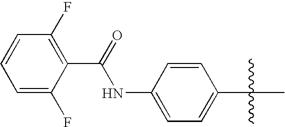 | 582 |
| 155 | Et | 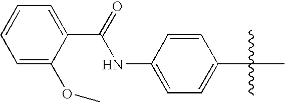 | 576 |
| 156 | Et | 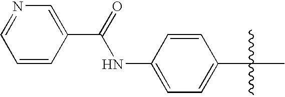 | 547 |
| 157 | Et | 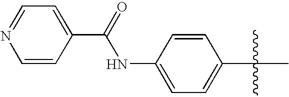 | 547 |
| 158 | Et | 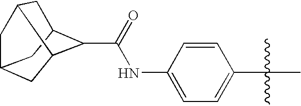 | 604 |
| 159 | Et | 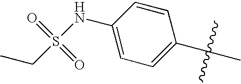 | 534 |
| 160 | Et | 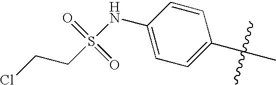 | 569 |
| 161 | Et | 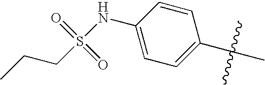 | 548 |
| 162 | Et | 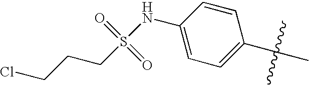 | 583 |
| 163 | Et | 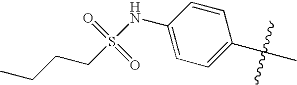 | 562 |
| 164 | Et | 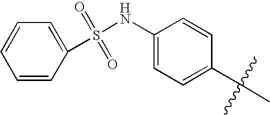 | 582 |
| 165 | Et | 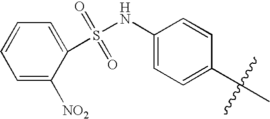 | 627 |
| 166 | Et | 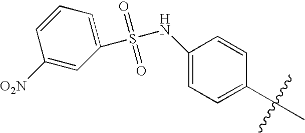 | 627 |
| 167 | Et | 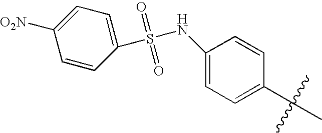 | 627 |
| 168 | Et | 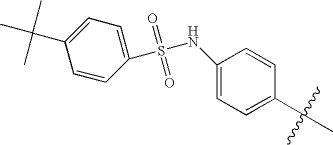 | 638 |
| 169 | Et | 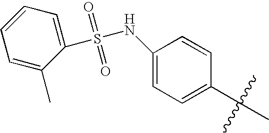 | 596 |
| 170 | Et | 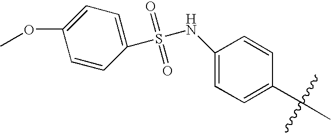 | 612 |
| 171 | Et | 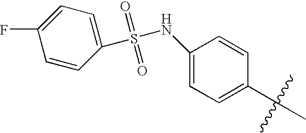 | 600 |
| 172 | Et | 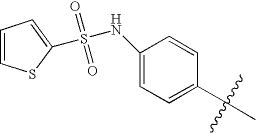 | 588 |
| 173 | Et | 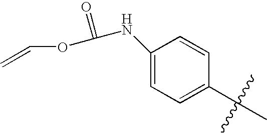 | 512 |
| 174 | Et | 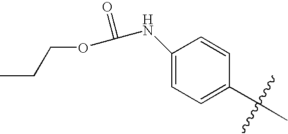 | 528 |
| 175 | Et | 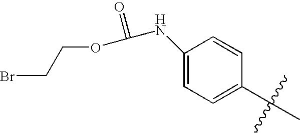 | 593 |
| 176 | Et | 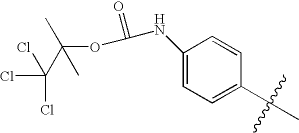 | 646 |
| 177 | Et | 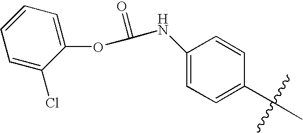 | 597 |
| 178 | Et | 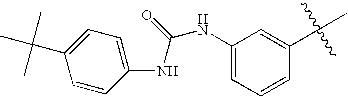 | 617 |
| 179 | Et | 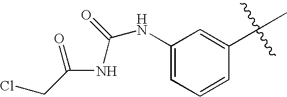 | 562 |
| 180 | Et | 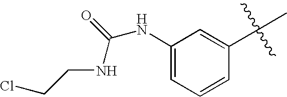 | 548 |
| 181 | Et | 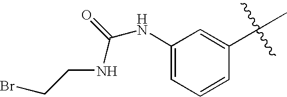 | 592 |
| 182 | Et | 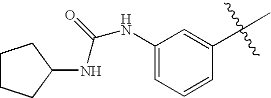 | 553 |
| 183 | Et | 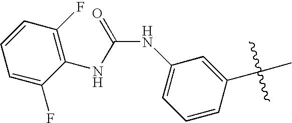 | 597 |
| 184 | Et | 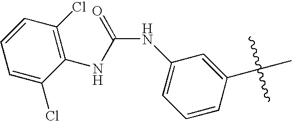 | 630 |
| 185 | Et | 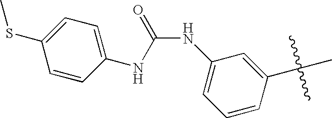 | 607 |
| 186 | Et | 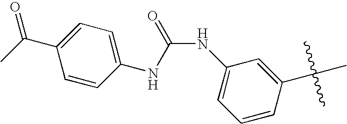 | 603 |
| 187 | Et | 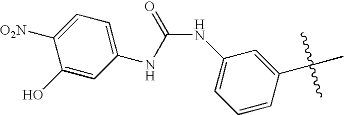 | 488 |
| 188 | Et | 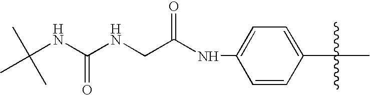 | 598 |
| 189 | Et | 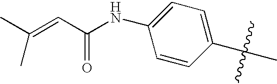 | 524 |
| 190 | Et | 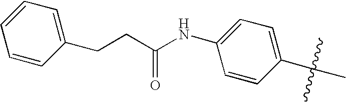 | 574 |
| 191 | Et | 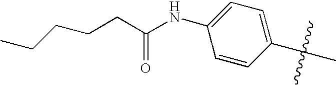 | 540 |
| 192 | Et | 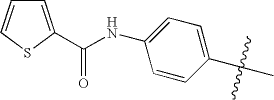 | 552 |
| 193 | Et | 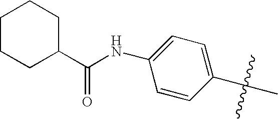 | 552 |
| 194 | Et | 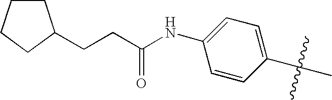 | 566 |
| 195 | Et | 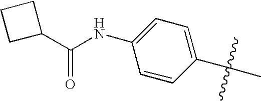 | 524 |
| 196 | Et | 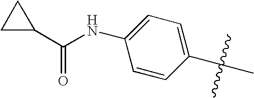 | 510 |
| 197 | Et | 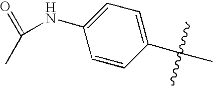 | 484 |
| 198 | Et | 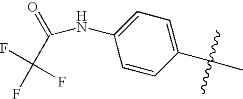 | 538 |
| 199 | Et | 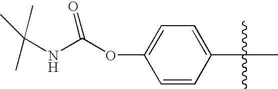 | 542 |
| 200 | Et | 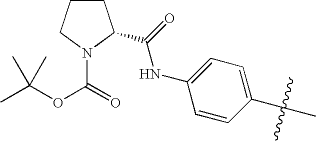 | 639 |
| 201 | Et | 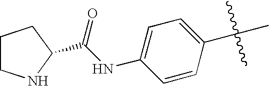 | 539 |
| 202 | t-Bu | 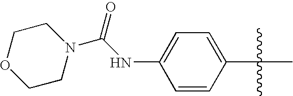 | 583 |
| 203 | t-Bu | 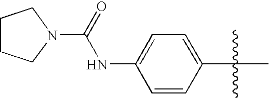 | 567 |
| 204 | t-Bu | 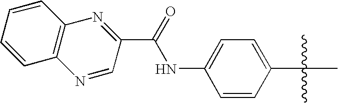 | 626 |
| 205 | t-Bu | 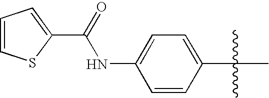 | 580 |
| 206 | t-Bu | 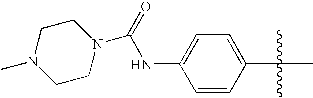 | 596 |
| 207 | t-Bu | 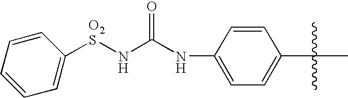 | 596 |
| 208 | t-Bu | 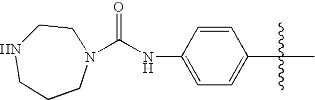 | 596 |
| 209 | t-Bu | 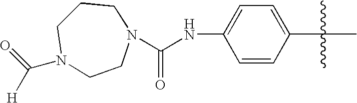 | 624 |
| 210 | t-Bu | 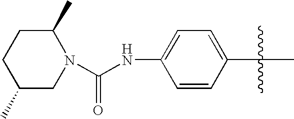 | 609 |
| 211 | t-Bu | 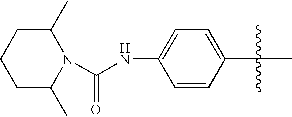 | 609 |
| 212 | t-Bu | 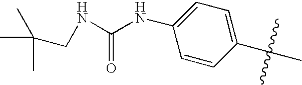 | 583 |
| 213 | t-Bu | 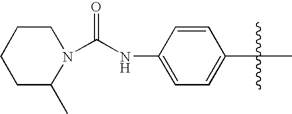 | 595 |
| 214 | t-Bu | 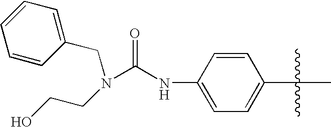 | 647 |
| 215 | t-Bu | 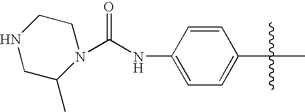 | 596 |
| 216 | t-Bu | 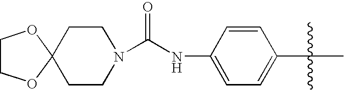 | 639 |
| 217 | t-Bu | 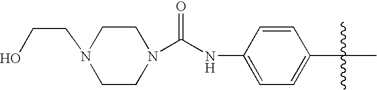 | 641 |
| 218 | t-Bu | 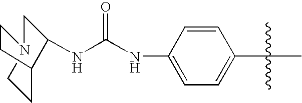 | 622 |
| 219 | t-Bu | 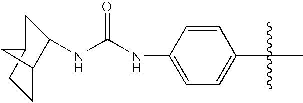 | 607 |
| 220 | t-Bu | 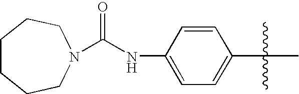 | 595 |
| 221 | t-Bu | 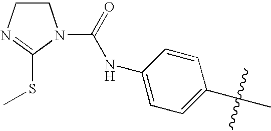 | 612 |
| 222 | t-Bu | 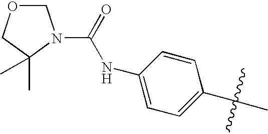 | 597 |
| 223 | t-Bu | 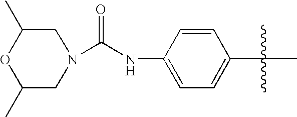 | 611 |
| 224 | t-Bu | 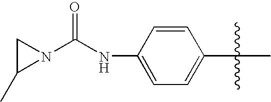 | 553 |
| 225 | t-Bu | 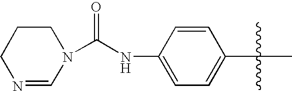 | 580 |
| 226 | t-Bu | 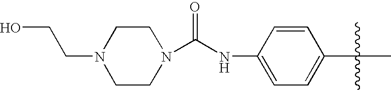 | 626 |
| 227 | t-Bu | 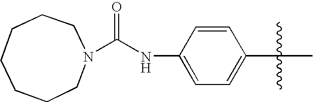 | 609 |
| 228 | t-Bu | 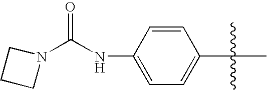 | 553 |
| 229 | t-Bu | 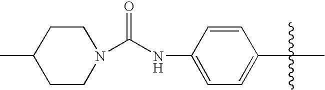 | 595 |
| 230 | t-Bu | 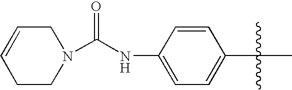 | 579 |
| 231 | t-Bu | 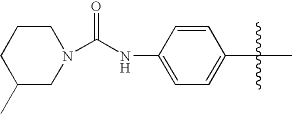 | 595 |
| 232 | t-Bu | 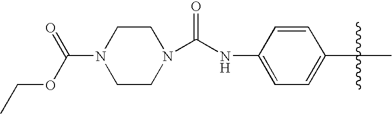 | 554 |

### Example 233

### 3-(2-Chloro-ethoxy)benzamide

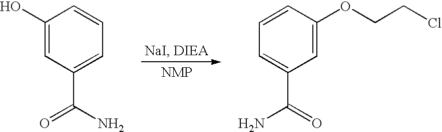

A mixture of 3-hydroxybenzamide (2.7 g, 19.7 mmol, prepared by treatment of the methyl ester with aqueous ammonium hydroxide), 1,2-dichloroethane (50 mL) and anhydrous potassium carbonate (6 g, 43.5 mmol) in acetonitrile (150 mL) were heated at reflux for 5 days. The mixture was cooled to room temperature, insolubles removed by filtration and volatiles removed on a rotary evaporator. The product was precipitated from chloroform-ether to give 0.5 g of 3-(2-chloroethoxy)benzamide.

### 4-(2-{4-[2-(4-Ethylphenyl)-1H-benzimidazol-4-yl]-piperazin-1-yl}-ethoxy)benzamide

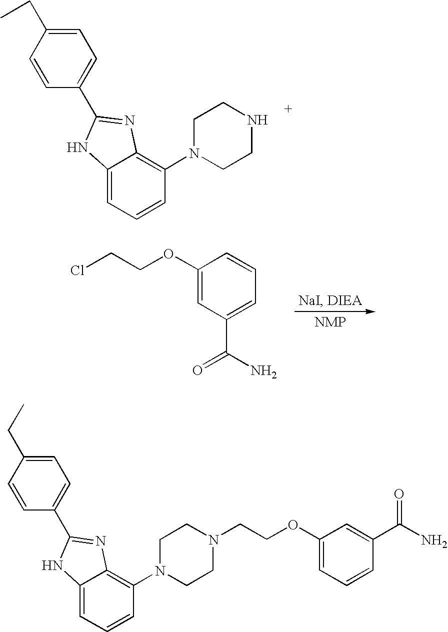

A mixture of 4-ethylphenylbenzimidazolyl piperazine (0.49 g, 1.6 mmol), meta-(2-chloroethoxy)benzamide (0.29 g, 1,45 mmol), diisopropylethylamine amine (2.5 mL, 14.4 mmol), and sodium iodide (0.3 g, 13.3 mmol) in N-methylpyrrolidinone (5 mL) were heated in a 65° C. oil bath for 5 days. The reaction mixture was partitioned between chloroform and water, and the organic phase concentrated on a rotary evaporator. The crude product was dissolved in methanol, filtered and chromatographed on a Gilson HPLC(Method E) to give 0.24 g (35%) of the title compound, as the TFA salt (2 eq., inferred from combustion data), a tan powder. HPLC (column; Xterra MS, C18, 3.5 μm, 4.6×50 mm; 5/95-95/5, 10 mins., hold for 2.5 mins., A=acetonitrile, B=PIC-B-6) rt=4.7 mins, (99.5% @ 210 nm). M/z=469, [M−H]−

### 5-(2-Chloro-ethoxy)-2-hydroxy-benzamide

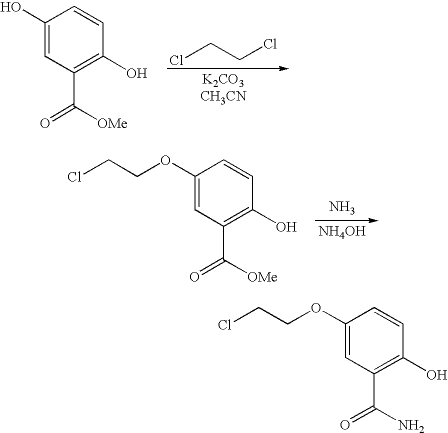

A mixture of methyl 2,5-dihydroxybenzoate (5 g, 0.03 mole), 1,2-dichloroethane (30 ml), potassium carbonate (8.3 g, 0.06 mole), and acetonitrile (225 mL) were heated in an 85° C. oil bath for 4 days. The reaction mixture was filtered and concentrated on a rotary evaporator to give 6.78 g of 5-(2-Chloro-ethoxy)-2-hydroxy-benzoic acid methyl ester, as a white wax. NMR and analytical HPLC/MS were consistent with the indicated structure. 1.5 g of this material were added to a mixture of 2M ammonia solution in methanol and aqueous ammonium hydroxide (28%). After several days at ambient temperature HPLC/MS indicated conversion was complete. The reaction mixture was concentrated (cold), triturated with warm chloroform and collected on a Buchner funnel to give 1.3 g of 5-(2-Chloro-ethoxy)-2-hydroxy-benzamide.

### tert-Butyl-carbamic acid 2-carbamoyl-4-(2-chloro-ethoxy)-phenyl ester

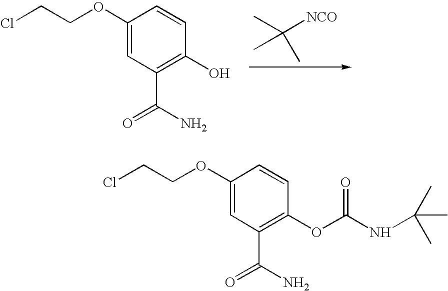

A mixture of 5-(2-Chloro-ethoxy)-2-hydroxy-benzamide (0.6 g, 2.78 mmol), diisopropylethylamine amine (0.5 mL, 1 eq.), 1.32 mL (4.1 eq.) of t-butylisocyanate and 1.27 g (2.78 mmol) sodium t-butoxide were heated at 60° C. for 60 h. The mixture was cooled to room temperature and poured into water, acidified with 1N HCl and extracted with ether. The extracts were dried (MgSO₄), filtered and concentrated to give 0.8 g of the above title compound. LC/MS; m/z=314.

### Example 234

### tert-Butyl-carbamic acid 2-carbamoyl-4-(2-{4-[2-(4-ethyl-phenyl)-1H-benzoimidazol-4-yl]-piperazin-1-yl}-ethoxy)-phenyl ester

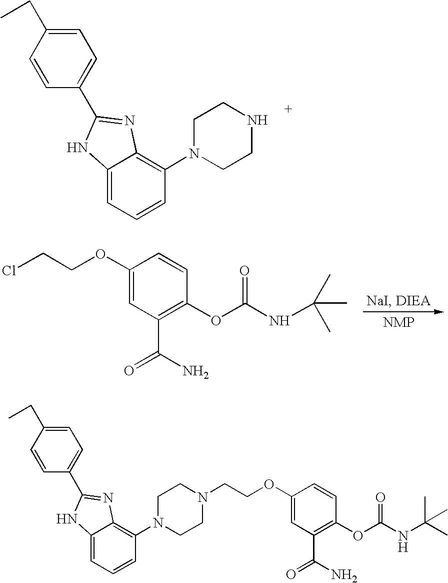

A mixture of 4-ethylphenylbenzimidazolyl piperazine (0.39 g, 1.27 mmol), chloroethoxyphenyl carbamate (0.23 g, 0.73 mmol), diisopropylethylamine (4 mL, 22 mmol) and sodium iodide (0.4 g, 2.22 mmol) in N-methylpyrrolidinone (4 mL) was heated in a 50° C. oil bath for 7 days. The mixture was cooled and poured into ether-water mixture. The organic layer was concentrated and chromatographed directly on a Gilson HPLC. The semi-purified product was suspended in chloroform-methanol mixture and treated with aqueous sodium bicarbonate. The organic phase was concentrated on the rotary evaporator and dried under vacuum to give 18 mg of the title compound as a yellow wax. MS (ESI-NEG) [M−H]−=583. HPLC (column: Xterra MS C18, 3.5 μm, 4.6×50 mm; 5/95-95/5, 10 mins., hold for 2.5 mins., (acetonitrile/0.1% TFA) rt=6 mins. (73.3% @ 210 nm; 70.1% @ 254 nm).

### Example 235

### Isopropyl-carbamic acid 2-carbamoyl-4-(2-{4-[2-(4-ethyl-phenyl)-1H-benzoimidazol-4-yl]-piperazin-1-yl}-ethoxy)-phenyl ester

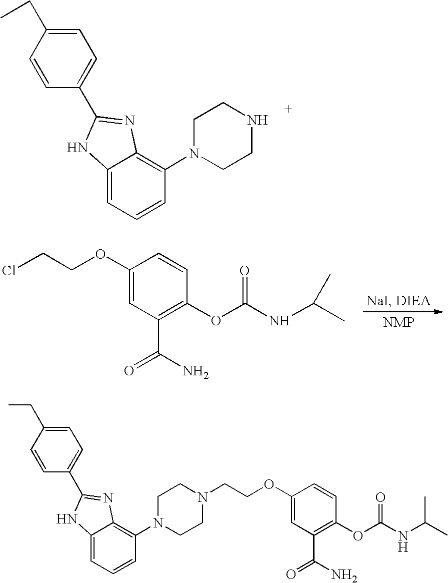

In like manner to example 234, Isopropyl-carbamic acid 2-carbamoyl-4-(2-{4-[2-(4-ethyl-phenyl)-1H-benzoimidazol-4-yl]-piperazin-1-yl}-ethoxy)-phenyl ester was prepared, except that after Gilson HPLC the product (as a light tan powder) was analyzed directly. CHN data suggested inclusion of 2 moles TFA and one mole H2O. (ESI-NEG) [M−H]−=569. HPLC (column: Xterra MS C18, 3.5 μm, 4.6×50 mm; 5/95-95/5, 10 mins., hold for 2.5 mins., (acetonitrile/0.1% TFA) rt=6 mins. (95.7% @ 210 nm; 96.6% @ 254 nm).

### Example 236

### Isopropyl-carbamic acid 4-(2-{4-[2-(4-tert-butyl-phenyl)-1H-benzoimidazol-4-yl]-piperazin-1-yl}-ethoxy)-2-carbamoyl-phenyl ester

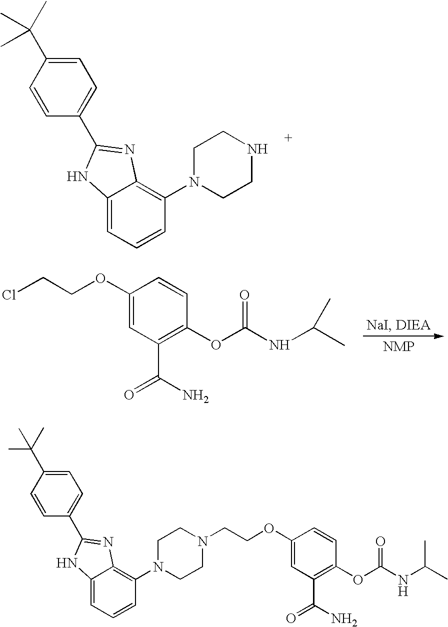

In a like manner to example 234, except that tert-butyl replaced ethyl, Isopropyl-carbamic acid 4-(2-{4-[2-(4-tert-butyl-phenyl)-1H-benzoimidazol-4-yl]-piperazin-1-yl}-ethoxy)-2-carbamoyl-phenyl ester was obtained, as a tan powder. CHN data suggested inclusion of 2 moles TFA and one mole H₂O. (ESI-NEG) [M−H]−=597. HPLC (column: Xterra MS C18, 3.5 μm, 4.6×50 mm; 5/95-95/5, 10 mins., hold for 2.5 mins., (acetonitrile/0.1% TFA) rt=6 mins. (88.7% @ 210 mm; 89.4% @ 254 nm).

### 6-(2-Chloro-ethoxy)-benzo[e][1,3]oxazine-2,4-dione

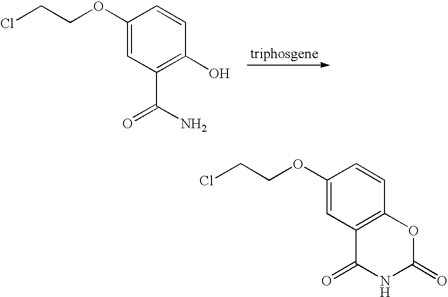

A slurry of hydroxy amide (2.5 g, 11 mmol) in chloroform (100 mL) was cooled in a −78° C. bath under N₂. To this mixture was added diisopropylethylamine (4 mL) and a solution of triphosgene (1.2 g, 4.05 mmol) dropwise. The reaction mixture was then allowed to warm gradually to room temperature. Insoluble matter was filtered off and the solution treated with MgSO₄, filtered and concentrated to a light brown solid. Trituration of this material with ether (100 mL) and filtration gave the oxazine dione (0.88 g) as a waxy off-white solid.

### Example 237

### 6-(2-{4-[2-(4-tert-Butyl-phenyl)-1H-benzoimidazol-4-yl]-piperazin-1-yl}-ethoxy)-benzo[e][1,3]oxazine-2,4-dione

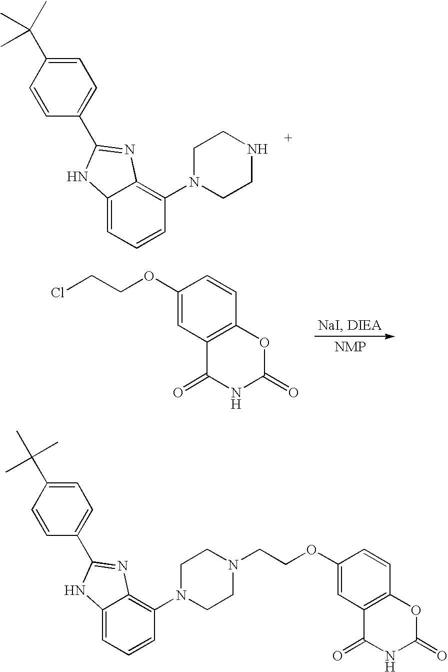

In like manner to example 236, 6-(2-{4-[2-(4-tert-Butyl-phenyl)-1H-benzoimidazol-4-yl]-piperazin-1-yl}-ethoxy)-benzo[e][1,3]oxazine-2,4-dione was prepared. Purification was carried out on a Gilson HPLC to give the title compound as a white powder. (ESI-NEG) [M−H]−=538. HPLC (column: Xterra MS C18, 3.5 μm, 4.6×50 mm; 5/95-95/5, 10 mins., hold for 2.5 mins., (acetonitrile/PIC B-5) rt=6 mins. (77.4% @ 210 nm; 75.7% @ 254 nm).

### Example 238

### 4-(2-{4-[2-(4-Ethyl-phenyl)-1H-benzoimidazol-4-yl]-piperazin-1-yl}-ethoxy)-1H-benzoimidazol-2-yl-cyanamide

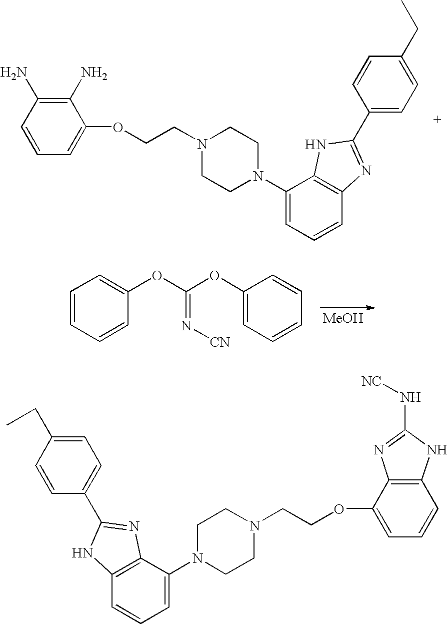

A mixture of 3-(2-{4-[2-(4-ethyl-phenyl)-3H-benzoimidazol-4-yl]-piperazin-1-yl}-ethoxy)-benzene-1,2-diamine (50 mg, 0.11 mmol), diphenyl cyanocarbonimidate (26 mg, 0.11 mmol) was stirred at room temperature overnight. Volatile material was removed on a rotary evaporator and the black viscous oil was stirred overnight with ether. Insolubles were collected on a Buchner funnel, washed with ether and air dried to give the title compound, as a brown powder. LCMS (Method A) ˜83% pure, rt=0.85 min. (ESI-NEG) [M−H]−=505, (ESI-POS) [M+H]+=507.

### Example 239

### 4-(2-{4-{2-(4-tert-Butyl-phenyl)-1H-benzoimidazol-4-yl}-piperazin-1-yl}-ethoxy)-1,3-dihydro-benzoimidazol-2-ylideneamine

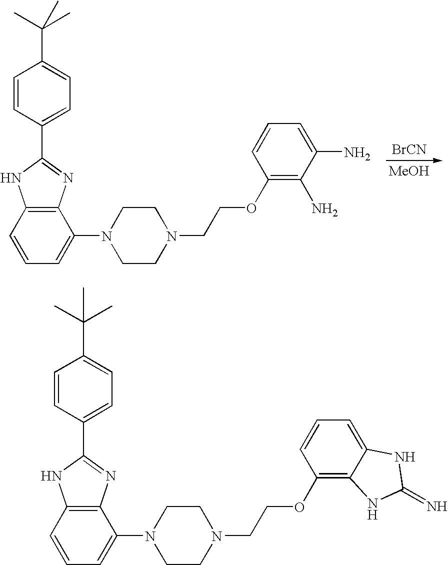

To a solution of 3-(2-{4-[2-(4-tert-Butyl-phenyl)-1H-benzoimidazol-4-yl]-piperazin-1-yl}-ethoxy)-benzene-1,2-diamine (484 mg, 0.62 mmol) in methanol (5 mL) was added cyanogen bromide (0.25 mL, 0.74 mmol, 3M solution in DCM) dropwise at room temperature. After 2 hours, NaOH (0.5 mL, 1N) was added and the mixture left to stir overnight. Volatile materials were removed on a rotary evaporator and the residue was chromatographed on silica gel, elution with 4% MeOH-DCM, followed by the same +3% NH₄OH to give 67 mg of the title compound as a light brown powder. NMR; consistant, LCMS (Method A) 98%, rt=1.09 min., (ESI-NEG) [M−H]−=508.

### Example 240

### 1-[4-(2-[2-(4-tert-Butyl-phenyl)-1H-benzoimidazol-4-yl]-piperazin-1-yl}-ethoxy)-2-imino-2,3-dihydro-benzoimidazol-1-yl}-2,2-dimethyl-propan-1-one

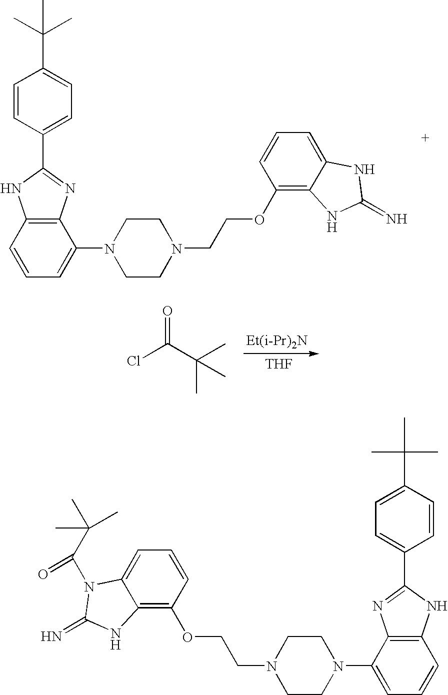

To a mixture of 4-(2-{4-[2-(4-tert-butyl-phenyl)-1H-benzoimidazol-4-yl]-piperazin-1-yl}-ethoxy)-1,3-dihydro-benzoimidazol-2-ylideneamine (example 239, 50 mg, 0.098 mmol), Hunig's base (25 μL) in THF (0.5 mL) was added 2,2-dimethyl-propionyl chloride (neat). After several days at room temperature, water and EtOAc were added, and the organic phase was washed sequentially with water and saturated aqueous brine solution, dried over MgSO₄, and concentrated. The product was purified on a Gilson HPLC (Method E) to give the title compound (2 TFA salt, 3.5 mg), as a tan powder. LCMS (Method A) ˜87% pure, rt=1.43 min. (ESI-NEG) [M−H]−=592, (ESI-POS) [M+H]+=594.

### Example 241

### 1-{4-(2-{4-{2-(4-tert-Butyl-phenyl)-1H-benzoimidazol-4-yl]-piperazin-1-yl}-ethoxy)-2-imino-2,3-dihydro-benzoimidazol-1-yl]-propan-1-one

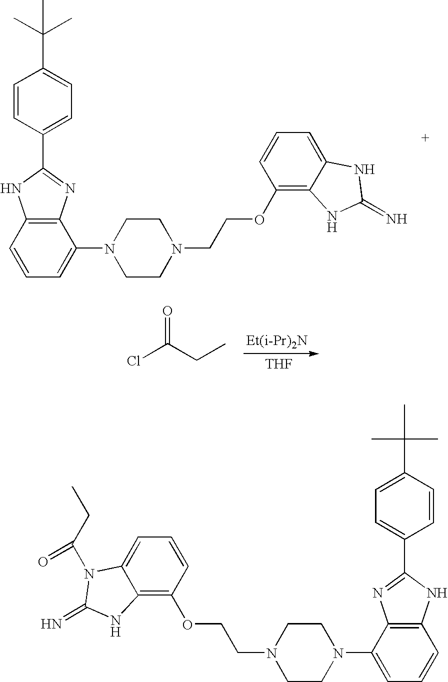

In like manner to example 240, the title compound was prepared from example 239 and propionyl chloride. The product was purified on a Gilson HPLC (Method E) to give 241 (2 TFA salt), as an off-white powder. LCMS (Method A) ˜87% pure, rt=1.20 min. (ESI-NEG) [M−H]−=562, (ESI-POS) [M+H]+=564.

### Example 242

### 4-(2-{4-[2-(4-tert-Butyl-phenyl)-1H-benzoimidazol-4-yl]-piperazin-1-yl}-ethoxy)-2-imino-2,3-dihydro-benzoimidazole-1-carboxylic acid ethyl ester

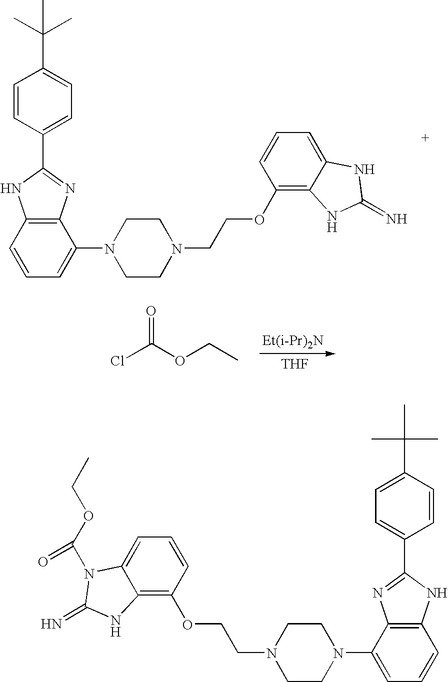

In like manner to example 240 (except that the reaction mixture was heated in a 30° C. bath for 30 min, then stirred at room temperature for 2.5 days), the title compound was prepared from example 239 and ethyl chloroformate. The product was purified on a Gilson HPLC (Method E) to give 10 (2 TFA salt), as an off-white solid. LCMS (Method A) ˜85% pure, rt=1.23 min. (ESI-NEG) [M−H]−=580, (ESI-POS) [M+H]+=582.

### Example 243

Biological Activity

COS cell membranes containing human GnRH receptors were incubated with radioactively labeled D-trp6 LHRH in the presence of increasing concentrations of compounds of the present invention. Membrane bound radioactivity was measured after separating the free radioactivity by filtration method, and IC₅₀ values were calculated using SAS analysis system. The methods are well known, and described, for example, in Receptor-binding affinity of gonadotropin-releasing hormone analogs:analysis by radioligand-receptor assay. Endocrinology, 1980, 106:1154-1159.

All compounds have hGnRH binding IC₅₀'s between 1 and 10,000 nM.

Various modifications of the invention, in addition to those described herein, will be apparent to those skilled in the art from the foregoing description. Such modifications are also intended to fall within the scope of the appended claims.

What is claimed:

1. A compound of Formula I: 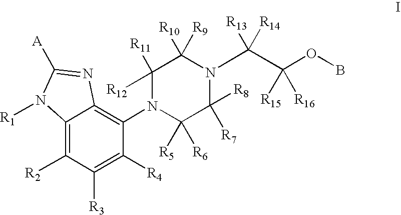
or a pharmaceutically acceptable salt thereof, wherein:
A is optionally substituted cycloalkyl, aryl, heteroaryl, or diaryl substituted alkyl;
B is optionally substituted aryl or heteroaryl;
R₁ is H, the tautomeric form, or optionally substituted alkyl;
R₂, R₃, and R₄ are, independently, H, optionally substituted alkyl, halogen, or OR₁; and
R₅, R₆, R₇, R₈, R₉, R₁₀, R₁₁, R₁₂, R₁₃, R₁₄, R₁₅, and R₁₆, are, independently, H or alkyl, alkenyl, or alkynyl, each alkyl, alkenyl, or alkynyl being optionally substituted.

2. The compound of claim 1, wherein B is: 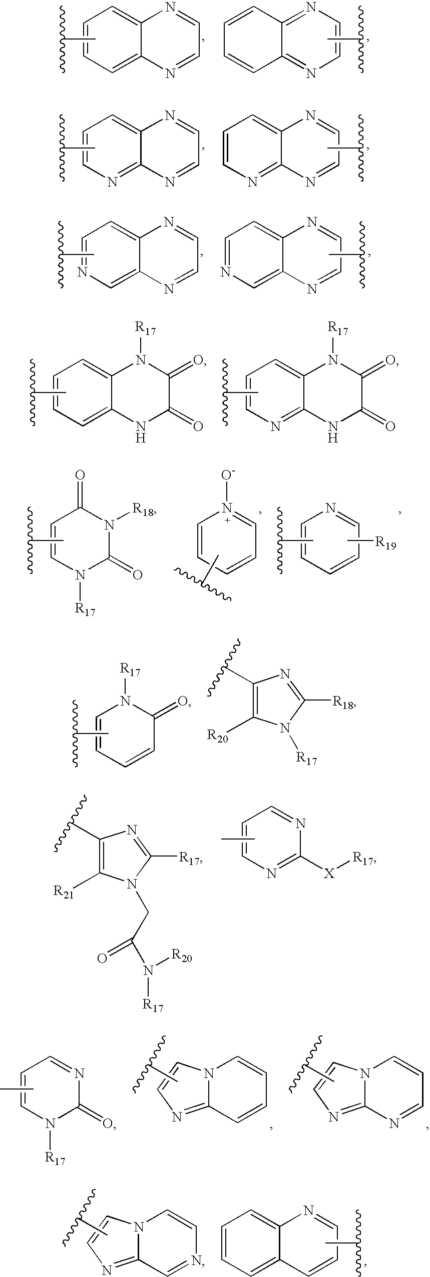 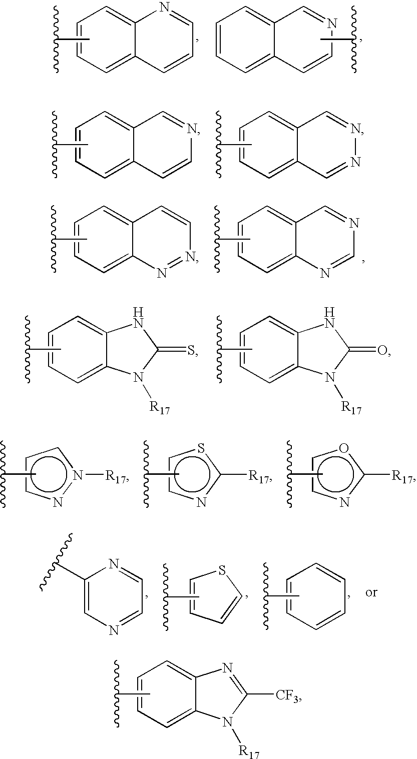
each B also having up to three R₂₀ substituents attached to the ring of B containing at least one N; wherein:
R₁₇ is hydrogen, alkyl, alkenyl, alkynyl, aryl, heteroaryl, arylalkyl, heteroarylalkyl, R₂₂XR₂₃, COXR₂₂, or XR₂₂, wherein X is O, NR₂₃, S, SO, or SO₂;
R₁₈ is hydrogen, alkyl, alkenyl, alkynyl, CO₂R₂₂, or CONR₂₂R₂₃;
R₁₉ is hydrogen, CO₂R₂₂, CONR₂₂R₂₃, S, SR₂₂, SO₂, SO₂R₂₂, or SO₃;
R₂₀ and R₂₁ are, independently, H, alkyl, alkenyl, or alkynyl; and
R₂₂ and R₂₃ are, independently, H or alkyl, alternatively R₂₂ and R₂₃, taken together with the atoms to which they are attached, form a 3-7 membered heterocycle, having 1-3 heteroatoms selected from N, O, and S.

3. The compound of claim 1, wherein B is of Formula II: 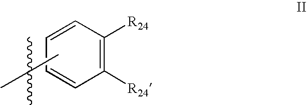
wherein:
R₂₄ and R₂₄′ are, independently, H, optionally substituted alkyl, halogen, NO₂, NHR₂₅, CONHR₂₅, OCONHR₂₅, NHCON(R₂₅)₂, NHCONHCOR₂₅, NHCOR₂₅, NHCO₂R₂₅, NHSO₂R₂₅, OH;
alternatively R₂₄ and R₂₄′, taken together with the atoms to which they are attached, form an optionally substituted 3-7 membered heterocycle, having 1-3 heteroatoms selected from N, O, and S; and
R₂₅ is, independently, H, CF₃, O-alkyl, alkyl, alkenyl, cycloalkyl, heterocycloalkyl, aryl, heteroaryl, arylalkyl, heteroarylalkyl, or CHNHCONH-alkyl; wherein any R₂₄, R₂₄′, or R₂₅ group is optionally substituted.

4. The compound of claim 3, wherein B is of Formula III: 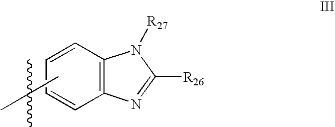
or a tautomeric form thereof; wherein:
R₂₆ is alkyl, S, SR₂₇, CF₃, NH, or NHR₂₇;
R₂₇ is, independently, H, alkyl, CN, CO₂R₂₈, or C(═O)R₂₈; and
R₂₈ is alkyl.

5. The compound of claim 1, wherein A or B is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃₁ and R₃₂, taken together with the atoms to which they are attached, form a 3-7 membered heterocycle, having 1-3 heteroatoms selected from N, O, and S.

6. The compound of claim 3, wherein R₂₄ or R₂₄′ is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃₁ and R₃₂, taken together with the atoms to which they are attached, form a 3-7 membered heterocycle, having 1-3 heteroatoms selected from N, O, and S.

7. The compound of claim 4, wherein R₂₆ or R₂₇ is substituted with at least one of alkyl, alkenyl, alkynyl, alkoxy, cycloalkyl, heteroalkyl, heterocycloalkyl, aryl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃₁ and R₃₂ are, independently, H or alkyl, alternatively R₂₉ and R₃₀ or R₃₁ and R₃₂, taken together with the atoms to which they are attached, form a 3-7 membered heterocycle, having 1-3 heteroatoms selected from N, O, and S.

8. The compound of claim 1, wherein B is 4-[2-thiobenzimidazolone], 4-[2-(trifluoromethyl)benzimidazole] or N-t-butylcarbamoyl-4-aminophenyl.

9. The compound of claim 1, wherein A is phenyl, naphthyl, thiophenyl, or pyridyl, each optionally substituted.

10. The compound of claim 1, wherein A is phenyl, 2-thiophenyl, 3-thiophenyl, 2-pyridyl, 3-pyridyl, or 4-pyridyl each optionally substituted.

11. The compound of claim 9, wherein A is substituted with at least one of alkyl, alkenyl, alkynyl, alkoxy, cycloalkyl, heteroalkyl, heterocycloalkyl, aryl, arylalkyl, aryloxy, heteroaryl, NR₂₉R₃₀, CF₃, NHCOR₂₉, COR₂₉, OR₂₉, S, SR₂₉, SO₂, SO₂R₂₉, SO₃, NO₂, CN, or halogen, wherein R₂₉ and R₃₀ are, independently, H, alkyl, alkenyl, alkynyl, alkoxy, aryl, amino, CF₃, or NR₃₁R₃₂, wherein R₃, and R₃₂ are, independently, H or alkyl, alternatively R₂₉ and R₃₀ or R₃, and R₃₂, taken together with the atoms to which they are attached, form a 3-7 membered heterocycle, having 1-3 heteroatoms selected from N, O, and S.

12. The compound of claim 1, wherein A is alkyl substituted phenyl.

13. The compound of claim 1, wherein A is ethyl substituted phenyl, 4-t-butylphenyl, 4-methanesulfonylphenyl, 4-N,N-diethylaminophenyl.

14. The compound of claim 1 that is 7-(2-{4-[2-(4-ethylphenyl)-1H-benzimidazol-4-yl]piperazin-1-yl}ethoxy)-1H-benzimidazol-2-ylcyanamide; ethyl 4-(2-{4-[2-(4-tert-butylphenyl)-1H-benzimidazol-4-yl]piperazin-1-yl}ethoxy)-2-imino-2,3-dihydro-1H-benzimidazole-1-carboxylate; 4-(2-{4-[2-(4-tert-butylphenyl)-1H-benzimidazol-4-yl]piperazin-1-yl}ethoxy)-1-propionyl-1,3-dihydro-2H-benzimidazol-2-imine; 4-(2-{4-[2-(4-tert-butylphenyl)-1H-benzimidazol-4-yl]piperazin-1-yl}ethoxy)-1-(2,2-dimethylpropanoyl)-1,3-dihydro-2H-benzimidazol-2-imine; 3-(4-{4-[4-(2-{[2-(trifluoromethyl)-1H-benzimidazol-4-yl]oxy}ethyl)piperazin-1-yl]-1H-benzimidazol-2-yl}benzyl)phenol; 2-(aminocarbonyl)-4-(2-{4-[2-(4-tert-butylphenyl)-1H-benzimidazol-4-yl]piperazin-1-yl}ethoxy)phenyl isopropylcarbamate; 2-(aminocarbonyl)-4-(2-{4-[2-(4-ethylphenyl)-1H-benzimidazol-4-yl]piperazin-1-yl}ethoxy)phenyl isopropylcarbamate; 6-(2-{4-[2-(4-tert-butylphenyl)-1H-benzimidazol-4-yl]piperazin-1-yl}ethoxy)-2H-1,3-benzoxazine-2,4(3H)-dione; 4-(2-{4-[2-(4-ethylphenyl)-1H-benzimidazol-4-yl]piperazin-1-yl}ethoxy)phenol; N-benzyl-N′-[4-(2-{4-[2-(4-tert-butylphenyl)-1H-benzimidazol-4-yl]piperazin-1-yl}ethoxy)phenyl]-N-(2-hydroxyethyl)urea; N-[4-(2-{4-[2-(4-tert-butylphenyl)-1H-benzimidazol-4-yl]piperazin-1-yl}ethoxy)phenyl]-2-methylpiperazine-1-carboxamide; N-[4-(2-{4-[2-(4-tert-butylphenyl)-1H-benzimidazol-4-yl]piperazin-1-yl}ethoxy)phenyl]-N′-neopentylurea; N-[4-(2-{4-[2-(4-tert-butylphenyl)-1H-benzimidazol-4-yl]piperazin-1-yl}ethoxy)phenyl]-2,6-dimethylpiperidine-1-carboxamide; (2S,5S)-N-[4-(2-{4-[2-(4-tert-butylphenyl)-1H-benzimidazol-4-yl]piperazin-1-yl}ethoxy)phenyl]-2,5-dimethylpiperidine-1-carboxamide; 2-(aminocarbonyl)-4-(2-{4-[2-(4-ethylphenyl)-1H-benzimidazol-4-yl]piperazin-1-yl}ethoxy)phenyl tert-butylcarbamate; N-[4-(2-{4-[2-(4-tert-butylphenyl)-1H-benzimidazol-4-yl]piperazin-1-yl}ethoxy)phenyl]-4-formyl-1,4-diazepane-1-carboxamide; N-[4-(2-{4-[2-(4-tert-butylphenyl)-1H-benzimidazol-4-yl]piperazin-1-yl}ethoxy)phenyl]-1,4-diazepane-1-carboxamide; N-({[4-(2-{4-[2-(4-tert-butylphenyl)-1H-benzimidazol-4-yl]piperazin-1-yl}ethoxy)phenyl]amino}carbonyl)benzenesulfonamide; N-[4-(2-{4-[2-(4-tert-butylphenyl)-1H-benzimidazol-4-yl]piperazin-1-yl}ethoxy)phenyl]-4-methylpiperazine-1-carboxamide; 3-(2-{4-[2-(4-ethylphenyl)-1H-benzimidazol-4-yl]piperazin-1-yl}ethoxy)benzamide; 2-(4,5,6,7-tetrahydro-1-benzothien-3-yl)-4-[4-(2-{[2-(trifluoromethyl)-1H-benzimidazol-4-yl]oxy}ethyl)piperazin-1-yl]-1H-benzimidazole; 2-(5-isopropylthien-2-yl)-4-[4-(2-{[2-(trifluoromethyl)-1H-benzimidazol-4-yl]oxy}ethyl)piperazin-1-yl]-1H-benzimidazole; 4-(2-{4-[2-(4-tert-butylphenyl)-1H-benzimidazol-4-yl]piperazin-1-yl}ethoxy)-1,3-dihydro-2H-benzimidazol-2-imine; N-[4-(2-{4-[2-(4-tert-butylphenyl)-1H-benzimidazol-4-yl]piperazin-1-yl}ethoxy)phenyl]quinoxaline-2-carboxamide; N-[4-(2-{4-[2-(4-tert-butylphenyl)-1H-benzimidazol-4-yl]piperazin-1-yl}ethoxy)phenyl]thiophene-2-carboxamide; N-[4-(2-{4-[2-(4-tert-butylphenyl)-1H-benzimidazol-4-yl]piperazin-1-yl}ethoxy)phenyl]pyrrolidine-1-carboxamide; N-[4-(2-{4-[2-(4-tert-butylphenyl)-1H-benzimidazol-4-yl]piperazin-1-yl}ethoxy)phenyl]morpholine-4-carboxamide; 4-(2-{4-[2-(4-ethylphenyl)-1H-benzimidazol-4-yl]piperazin-1-yl}ethoxy)benzamide; N-[4-(2-{4-[2-(4-ethylphenyl)-1H-benzimidazol-4-yl]piperazin-1-yl}ethoxy)phenyl]-L-prolinamide; tert-butyl (2S)-2-({[4-(2-{4-[2-(4-ethylphenyl)-1H-benzimidazol-4-yl]piperazin-1-yl}ethoxy)phenyl]amino}carbonyl)pyrrolidine-1-carboxylate; 4-(2-{4-[2-(4-ethylphenyl)-1H-benzimidazol-4-yl]piperazin-1-yl}ethoxy)phenyl tert-butylcarbamate; 2-(5-tert-butylthien-3-yl)-4-[4-(2-{[2-(trifluoromethyl)-1H-benzimidazol-4-yl]oxy}ethyl)piperazin-1-yl]-1H-benzimidazole; 2-(5-ethylthien-3-yl)-4-[4-(2-{[2-(trifluoromethyl)-1H-benzimidazol-4-yl]oxy}ethyl)piperazin-1-yl]-1H-benzimidazole; N2-[(tert-butylamino)carbonyl]-N-1-[4-(2-{4-[2-(4-ethylphenyl)-1H-benzimidazol-4-yl]piperazin-1-yl}ethoxy)phenyl]glycinamide; 5-(2-{4-[2-(4-ethylphenyl)-1H-benzimidazol-4-yl]piperazin-1-yl}ethoxy)-2-nitrophenol; 4-(2-{4-[2-(4-tert-butylphenyl)-1H-benzimidazol-4-yl]piperazin-1-yl}ethoxy)aniline; N-(4-tert-butylphenyl)-N′-[4-(2-{4-[2-(4-ethylphenyl)-1H-benzimidazol-4-yl]piperazin-1-yl}ethoxy)phenyl]urea; 5-(2-{4-[2-(4-ethylphenyl)-1H-benzimidazol-4-yl]piperazin-1-yl}ethoxy)-2-hydroxybenzamide; 2-(4-benzylphenyl)-4-[4-(2-{[2-(trifluoromethyl)-1H-benzimidazol-4-yl]oxy}ethyl)piperazin-1-yl]-1H-benzimidazole; 2-(5-tert-butylthien-2-yl)-4-[4-(2-{[2-(trifluoromethyl)-1H-benzimidazol-4-yl]oxy}ethyl)piperazin-1-yl]-1H-benzimidazole; N-(tert-butyl)-N′-[4-(2-{4-[2-(4-tert-butylphenyl)-1H-benzimidazol-4-yl]piperazin-1-yl}ethoxy)phenyl]urea; N-(tert-butyl)-N′-[3-(2-{4-[2-(4-ethylphenyl)-1H-benzimidazol-4-yl]piperazin-1-yl}ethoxy)phenyl]urea; 3-(2-{4-[2-(4-ethylphenyl)-1H-benzimidazol-4-yl]piperazin-1-yl}ethoxy)aniline; 2-(4-ethylphenyl)-4-{4-[2-(4-nitrophenoxy)ethyl]piperazin-1-yl}-1H-benzimidazole; 2-(4-ethylphenyl)-4-{4-[2-(3-nitrophenoxy)ethyl]piperazin-1-yl}-1H-benzimidazole; N-[4-(2-{4-[2-(4-ethylphenyl)-1H-benzimidazol-4-yl]piperazin-1-yl}ethoxy)phenyl]-2,2-dimethylpropanamide; N-[4-(2-{4-[2-(4-Ethyl-phenyl)-1H-benzoimidazol-4-yl]-piperazin-1-yl}-ethoxy)-phenyl]-2,2-dimethyl-propionamide; N-[4-(2-{4-[2-(4-ethylphenyl)-1H-benzimidazol-4-yl]piperazin-1-yl}ethoxy)phenyl]methanesulfonamide; N-[4-(2-{4-[2-(4-ethylphenyl)-1H-benzimidazol-4-yl]piperazin-1-yl}ethoxy)phenyl]-3,3-dimethylbutanamide; tert-butyl 4-(2-{4-[2-(4-ethylphenyl)-1H-benzimidazol-4-yl]piperazin-1-yl}ethoxy)phenylcarbamate; 4-ethyl-N-[4-(2-{4-[2-(4-ethylphenyl)-1H-benzimidazol-4-yl]piperazin-1-yl}ethoxy)phenyl]benzamide; neopentyl 4-(2-{4-[2-(4-ethylphenyl)-1H-benzimidazol-4-yl]piperazin-1-yl}ethoxy)phenylcarbamate; [4-(2-{4-[2-(4-Ethyl-phenyl)-1H-benzoimidazol-4-yl]-piperazin-1-yl}-ethoxy)-phenyl]-carbamic acid 2,2-dimethyl-propyl ester; 4-(2-{4-[2-(4-ethylphenyl)-1H-benzimidazol-4-yl]piperazin-1-yl}ethoxy)aniline; N-(tert-butyl)-N′-[4-(2-{4-[2-(4-ethylphenyl)-1H-benzimidazol-4-yl]piperazin-1-yl}ethoxy)phenyl]urea; 4-{2-[4-(2-phenyl-1H-benzimidazol-7-yl)piperazin-1-yl]ethoxy}-1,3-dihydro-2H-benzimidazole-2-thione; ethyl 4-({[4-(2-{4-[2-(4-tert-butylphenyl)-1H-benzimidazol-4-yl]piperazin-1-yl}ethoxy)phenyl]amino}carbonyl)piperazine-1-carboxylate; N-[4-(2-J{4-[2-(4-tert-butylphenyl)-1H-benzimidazol-4-yl]piperazin-1-yl}ethoxy)phenyl]-3-methylpiperidine-1-carboxamide; 3,6-Dihydro-2H-pyridine-1-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-3,6-dihydropyridine-1 (2H)-carboxamide; N-[4-(2-{4-[2-(4-tert-butylphenyl)-1H-benzimidazol-4-yl]piperazin-1-yl}ethoxy)phenyl]-4-methylpiperidine-1-carboxamide; N-[4-(2-{4-[2-(4-tert-butylphenyl)-1H-benzimidazol-4-yl]piperazin-1-yl}ethoxy)phenyl]azetidine-1-carboxamide; N-[4-(2-{4-[2-(4-tert-butylphenyl)-1H-benzimidazol-4-yl]piperazin-1-yl}ethoxy)phenyl]azocane-1-carboxamide; N-[4-(2-{4-[2-(4-tert-butylphenyl)-1H-benzimidazol-4-yl]piperazin-1-yl}ethoxy)phenyl]-4-(2-hydroxyethyl)piperazine-1-carboxamide; N-[4-(2-{4-[2-(4-tert-butylphenyl)-1H-benzimidazol-4-yl]piperazin-1-yl}ethoxy)phenyl]-5,6-dihydropyrimidine-1 (4H)-carboxamide; N-[4-(2-{4-[2-(4-tert-butylphenyl)-1H-benzimidazol-4-yl]piperazin-1-yl}ethoxy)phenyl]-2-methylaziridine-1-carboxamide; 2,6-Dimethyl-morpholine-4-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-2,6-dimethylmorpholine-4-carboxamide; N-[4-(2-{4-[2-(4-tert-butylphenyl)-1H-benzimidazol-4-yl]piperazin-1-yl}ethoxy)phenyl]-4,4-dimethyl-1,3-oxazolidine-3-carboxamide; N-[4-(2-{4-[2-(4-tert-butylphenyl)-1H-benzimidazol-4-yl]piperazin-1-yl}ethoxy)phenyl]-2-(methylthio)-4,5-dihydro-1H-imidazole-1-carboxamide; N-[4-(2-{4-[2-(4-tert-butylphenyl)-1H-benzimidazol-4-yl]piperazin-1-yl}ethoxy)phenyl]azepane-1-carboxamide; N-[(1R,2S,4S)-bicyclo[2.2.1]hept-2-yl]-N′-[4-(2-{4-[2-(4-tert-butylphenyl)-1H-benzimidazol-4-yl]piperazin-1-yl}ethoxy)phenyl]urea; N-1-azabicyclo[2.2.2]oct-3-yl-N′-[4-(2-{4-[2-(4-tert-butylphenyl)-1H-benzimidazol-4-yl]piperazin-1-yl}ethoxy)phenyl]urea; N-[4-(2-{4-[2-(4-tert-butylphenyl)-1H-benzimidazol-4-yl]piperazin-1-yl}ethoxy)phenyl]-2-methylpiperidine-1-carboxamide; N-[4-(2-{4-[2-(4-tert-butylphenyl)-1H-benzimidazol-4-yl]piperazin-1-yl}ethoxy)phenyl]-N′-[4-(2-hydroxyethyl)piperazin-1-yl]urea; 1,4-Dioxa-8-aza-spiro[4.5]decane-8-carboxylic acid [4-(2-{4-[2-(4-tert-butyl-phenyl)-1H-benzoimidazol-4-yl]-piperazin-1-yl}-ethoxy)-phenyl]-amide; N-[4-(2-{4-[2-(4-tert-butylphenyl)-1H-benzimidazol-4-yl]piperazin-1-yl}ethoxy)phenyl]-1,4-dioxa-8-azaspiro[4.5]decane-8-carboxamide; N-azepan-1-yl-N′-[4-(2-{4-[2-(4-tert-butylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-2,2,2-trifluoroacetamide; N-[4-(2-{4-[2-(4-ethylphenyl)-1H-benzimidazol-4-yl]piperazin-1-yl}ethoxy)phenyl]acetamide; N-[4-(2-{4-[2-(4-ethylphenyl)-1H-benzimidazol-4-yl]piperazin-1-yl}ethoxy)phenyl]cyclopropanecarboxamide; N-[4-(2-{4-[2-(4-ethylphenyl)-1H-benzimidazol-4-yl]piperazin-1-yl}ethoxy)phenyl]cyclobutanecarboxamide; 3-cyclopentyl-N-[4-(2-{4-[2-(4-ethylphenyl)-1H-benzimidazol-4-yl]piperazin-1-yl}ethoxy)phenyl]propanamide; N-[4-(2-{4-[2-(4-ethylphenyl)-1H-benzimidazol-4-yl]piperazin-1-yl}ethoxy)phenyl]cyclohexanecarboxamide; N-[4-(2-{4-[2-(4-ethylphenyl)-1H-benzimidazol-4-yl]piperazin-1-yl}ethoxy)phenyl]thiophene-2-carboxamide; N-[4-(2-{4-[2-(4-ethylphenyl)-1H-benzimidazol-4-yl]piperazin-1-yl}ethoxy)phenyl]hexanamide; N-[4-(2-{4-[2-(4-ethylphenyl)-1H-benzimidazol-4-yl]piperazin-1-yl}ethoxy)phenyl]-3-phenylpropanamide; N-[4-(2-{4-[2-(4-ethylphenyl)-1H-benzimidazol-4-yl]piperazin-1-yl}ethoxy)phenyl]-3-methylbut-2-enamide; N-(4-acetylphenyl)-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-[4-(methylthio)phenyl]urea; N-(2,6-dichlorophenyl)-N′-[4-(2-{4-[2-(4-ethylphenyl)-1H-benzimidazol-4-yl]piperazin-1-yl}ethoxy)phenyl]urea; N-(2,6-difluorophenyl)-N′-[4-(2-{4-[2-(4-ethylphenyl)-1H-benzimidazol-4-yl]piperazin-1-yl}ethoxy)phenyl]urea; N-cyclopentyl-N′-[4-(2-{4-[2-(4-ethylphenyl)-1H-benzimidazol-4-yl]piperazin-1-yl}ethoxy)phenyl]urea; N-(2-bromoethyl)-N′-[4-(2-{4-[2-(4-ethylphenyl)-1H-benzimidazol-4-yl]piperazin-1-yl}ethoxy)phenyl]urea; N-(2-chloroethyl)-N′-[4-(2-{4-[2-(4-ethylphenyl)-1H-benzimidazol-4-yl]piperazin-1-yl}ethoxy)phenyl]urea; 2-chloro-N-({[4-(2-{4-[2-(4-ethylphenyl)-1H-benzimidazol-4-yl]piperazin-1-yl}ethoxy)phenyl]amino}carbonyl)acetamide; N-(tert-butyl)-N′-[4-(2-{4-[2-(4-ethylphenyl)-1H-benzimidazol-4-yl]piperazin-1-yl}ethoxy)-2-fluorophenyl]urea; N-(tert-butyl)-N′-[4-(2-{4-[2-(4-ethylphenyl)-1H-benzimidazol-4-yl]piperazin-1-yl}ethoxy)-2-methylphenyl]urea; N-(tert-butyl)-N′-[2-chloro-4-(2-{4-[2-(4-ethylphenyl)-1H-benzimidazol-4-yl]piperazin-1-yl}ethoxy)phenyl]urea; [4-(2-{4-[2-(4-ethylphenyl)-1H-benzimidazol-4-yl]piperazin-1-yl}ethoxy)-2-methylphenyl]amine; [4-(2-{4-[2-(4-ethylphenyl)-1H-benzimidazol-4-yl]piperazin-1-yl}ethoxy)-2-fluorophenyl]amine; [2-chloro-4-(2-{4-[2-(4-ethylphenyl)-1H-benzimidazol-4-yl]piperazin-1-yl}ethoxy)phenyl]amine; 2-(4-ethylphenyl)-4-{4-[2-(3-fluoro-4-nitrophenoxy)ethyl]piperazin-1-yl}-1H-benzimidazole; 2-(4-ethylphenyl)-4-{4-[2-(3-methyl-4-nitrophenoxy)ethyl]piperazin-1-yl}-1H-benzimidazole; 2-chlorophenyl [4-(2-{4-[2-(4-ethylphenyl)-1H-benzimidazol-4-yl]piperazin-1-yl}ethoxy)phenyl]carbamate; 2,2,2-trichloro-1,1-dimethylethyl [4-(2-{4-[2-(4-ethylphenyl)-1H-benzimidazol-4-yl]piperazin-1-yl}ethoxy)phenyl]carbamate; 2-bromoethyl [4-(2-{4-[2-(4-ethylphenyl)-1H-benzimidazol-4-yl]piperazin-1-yl}ethoxy)phenyl]carbamate; propyl [4-(2-{4-[2-(4-ethylphenyl)-1H-benzimidazol-4-yl]piperazin-1-yl}ethoxy)phenyl]carbamate; vinyl [4-(2-{4-[2-(4-ethylphenyl)-1H-benzimidazol-4-yl]piperazin-1-yl}ethoxy)phenyl]carbamate; allyl [4-(2-{4-[2-(4-ethylphenyl)-1H-benzimidazol-4-yl]piperazin-1-yl}ethoxy)phenyl]carbamate; N-[4-(2-{4-[2-(4-ethylphenyl)-1H-benzimidazol-4-yl]piperazin-1-yl}ethoxy)phenyl]adamantane-1-carboxamide; N-[4-(2-{4-[2-(4-ethylphenyl)-1H-benzimidazol-4-yl]piperazin-1-yl}ethoxy)phenyl]isonicotinamide; N-[4-(2-{4-[2-(4-ethylphenyl)-1H-benzimidazol-4-yl]piperazin-1-yl}ethoxy)phenyl]nicotinamide; N-[4-(2-{4-[2-(4-ethylphenyl)-1H-benzimidazol-4-yl]piperazin-1-yl}ethoxy)phenyl]-2-methoxybenzamide; N-[4-(2-{4-[2-(4-ethylphenyl)-1H-benzimidazol-4-yl]piperazin-1-yl}ethoxy)phenyl]-2,6-difluorobenzamide; N-[4-(2-{4-[2-(4-ethylphenyl)-1H-benzimidazol-4-yl]piperazin-1-yl}ethoxy)phenyl]cyclopentanecarboxamide; N-[4-(2-{4-[2-(4-ethylphenyl)-1H-benzimidazol-4-yl]piperazin-1-yl}ethoxy)phenyl]-2-(trifluoromethyl)benzamide; 2-ethyl-N-[4-(2-{4-[2-(4-ethylphenyl)-1H-benzimidazol-4-yl]piperazin-1-yl}ethoxy)phenyl]butanamide; N-[4-(2-{4-[2-(4-ethylphenyl)-1H-benzimidazol-4-yl]piperazin-1-yl}ethoxy)phenyl]-2-methylbenzamide; 2,6-dichloro-N-[4-(2-{4-[2-(4-ethylphenyl)-1H-benzimidazol-4-yl]piperazin-1-yl}ethoxy)phenyl]benzamide; N-[4-(2-{4-[2-(4-ethylphenyl)-1H-benzimidazol-4-yl]piperazin-1-yl}ethoxy)phenyl]-2-(2-thienyl)acetamide; N-[4-(2-{4-[2-(4-ethylphenyl)-1H-benzimidazol-4-yl]piperazin-1-yl}ethoxy)phenyl]-2-furamide; N-[4-(2-{4-[2-(4-ethylphenyl)-1H-benzimidazol-4-yl]piperazin-1-yl}ethoxy)phenyl]-3-methylbutanamide; (2E)-N-[4-(2-{4-[2-(4-ethylphenyl)-1H-benzimidazol-4-yl]piperazin-1-yl}ethoxy)phenyl]but-2-enamide; N-[4-(2-{4-[2-(4-ethylphenyl)-1H-benzimidazol-4-yl]piperazin-1-yl}ethoxy)phenyl]acrylamide; N-[4-(2-{4-[2-(4-ethylphenyl)-1H-benzimidazol-4-yl]piperazin-1-yl}ethoxy)phenyl]propanamide; N-[4-(2-{4-[2-(4-ethylphenyl)-1H-benzimidazol-4-yl]piperazin-1-yl}ethoxy)phenyl]thiophene-2-sulfonamide; N-[4-(2-{4-[2-(4-ethylphenyl)-1H-benzimidazol-4-yl]piperazin-1-yl}ethoxy)phenyl]-4-fluorobenzenesulfonamide; N-[4-(2-{4-[2-(4-ethylphenyl)-1H-benzimidazol-4-yl]piperazin-1-yl}ethoxy)phenyl]-4-methoxybenzenesulfonamide; N-[4-(2-{4-[2-(4-ethylphenyl)-1H-benzimidazol-4-yl]piperazin-1-yl}ethoxy)phenyl]-2-methylbenzenesulfonamide; 4-tert-butyl-N-[4-(2-{4-[2-(4-ethylphenyl)-1H-benzimidazol-4-yl]piperazin-1-yl}ethoxy)phenyl]benzenesulfonamide; N-[4-(2-{4-[2-(4-ethylphenyl)-1H-benzimidazol-4-yl]piperazin-1-yl}ethoxy)phenyl]-4-nitrobenzenesulfonamide; N-[4-(2-{4-[2-(4-ethylphenyl)-1H-benzimidazol-4-yl]piperazin-1-yl}ethoxy)phenyl]-3-nitrobenzenesulfonamide; N-[4-(2-{4-[2-(4-ethylphenyl)-1H-benzimidazol-4-yl]piperazin-1-yl}ethoxy)phenyl]-2-nitrobenzenesulfonamide; N-[4-(2-{4-[2-(4-ethylphenyl)-1H-benzimidazol-4-yl]piperazin-1-yl}ethoxy)phenyl]benzenesulfonamide; N-[4-(2-{4-[2-(4-ethylphenyl)-1H-benzimidazol-4-yl]piperazin-1-yl}ethoxy)phenyl]butane-1-sulfonamide; 3-chloro-N-[4-(2-{4-[2-(4-ethylphenyl)-1H-benzimidazol-4-yl]piperazin-1-yl}ethoxy)phenyl]propane-1-sulfonamide; N-[4-(2-{4-[2-(4-ethylphenyl)-1H-benzimidazol-4-yl]piperazin-1-yl}ethoxy)phenyl]propane-2-sulfonamide; N-[4-(2-{4-[2-(4-ethylphenyl)-1H-benzimidazol-4-yl]piperazin-1-yl}ethoxy)phenyl]propane-1-sulfonamide; 2-chloro-N-[4-(2-{4-[2-(4-ethylphenyl)-1H-benzimidazol-4-yl]piperazin-1-yl}ethoxy)phenyl]ethanesulfonamide; N-[4-(2-{4-[2-(4-ethylphenyl)-1H-benzimidazol-4-yl]piperazin-1-yl}ethoxy)phenyl]ethanesulfonamide; N-[4-(2-{4-[2-(4-ethylphenyl)-1H-benzimidazol-4-yl]piperazin-1-yl}ethoxy)phenyl]-N′-(1,1,3,3-tetramethylbutyl)urea; N-[4-(2-{4-[2-(4-ethylphenyl)-1H-benzimidazol-4-yl]piperazin-1-yl}ethoxy)phenyl]-N′-(4-nitrophenyl)urea; N-[4-(2-{4-[2-(4-ethylphenyl)-1H-benzimidazol-4-yl]piperazin-1-yl}ethoxy)phenyl]-N′-(2-phenylethyl)urea; N-benzyl-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-(3-fluorophenyl)urea; N-[4-(2-{4-[2-(4-ethylphenyl)-1H-benzimidazol-4-yl]piperazin-1-yl}ethoxy)phenyl]-N′-(2-fluorophenyl)urea; N-[4-(2-{4-[2-(4-ethylphenyl)-1H-benzimidazol-4-yl]piperazin-1-yl}ethoxy)phenyl]-N′-(3-methylphenyl)urea; N-[4-(2-{4-[2-(4-ethylphenyl)-1H-benzimidazol-4-yl]piperazin-1-yl}ethoxy)phenyl]-N′-(2-methylphenyl)urea; N-(4-ethylphenyl)-N′-[4-(2-{4-[2-(4-ethylphenyl)-1H-benzimidazol-4-yl]piperazin-1-yl}ethoxy)phenyl]urea; N-cyclohexyl-N′-[4-(2-{4-[2-(4-ethylphenyl)-1H-benzimidazol-4-yl]piperazin-1-yl}ethoxy)phenyl]urea; N-allyl-N′-[4-(2-{4-[2-(4-ethylphenyl)-1H-benzimidazol-4-yl]piperazin-1-yl}ethoxy)phenyl]urea; ethyl ({[4-(2-{4-[2-(4-ethylphenyl)-1H-benzimidazol-4-yl]piperazin-1-yl}ethoxy)phenyl]amino}carbonyl)carbamate; N-butyl-N′-[4-(2-{4-[2-(4-ethylphenyl)-1H-benzimidazol-4-yl]piperazin-1-yl}ethoxy)phenyl]urea; N-[4-(2-{4-[2-(4-ethylphenyl)-1H-benzimidazol-4-yl]piperazin-1-yl}ethoxy)phenyl]-N′-isopropylurea; N-[4-(2-{4-[2-(4-ethylphenyl)-1H-benzimidazol-4-yl]piperazin-1-yl}ethoxy)phenyl]-N′-propylurea; N-ethyl-N′-[4-(2-{4-[2-(4-ethylphenyl)-1H-benzimidazol-4-yl]piperazin-1-yl}ethoxy)phenyl]urea; (3-{4-[4-(2-{[2-(trifluoromethyl)-1H-benzimidazol-4-yl]oxy}ethyl)piperazin-1-yl]-1H-benzimidazol-2-yl}phenyl)amine; 2-pyridin-4-yl-4-[4-(2-{[2-(trifluoromethyl)-1H-benzimidazol-4-yl]oxy}ethyl)piperazin-1-yl]-1H-benzimidazole; 2-(2,4-dimethoxyphenyl)-4-[4-(2-{[2-(trifluoromethyl)-1H-benzimidazol-4-yl]oxy}ethyl)piperazin-1-yl]-1H-benzimidazole; 2-(2,4-dichlorophenyl)-4-[4-(2-{[2-(trifluoromethyl)-1H-benzimidazol-4-yl]oxy}ethyl)piperazin-1-yl]-1H-benzimidazole; 2-methoxy-5-{4-[4-(2-{[2-(trifluoromethyl)-1H-benzimidazol-4-yl]oxy}ethyl)piperazin-1-yl]-1H-benzimidazol-2-yl}phenol; 2-(2,4-dimethylphenyl)-4-[4-(2-{[2-(trifluoromethyl)-1H-benzimidazol-4-yl]oxy}ethyl)piperazin-1-yl]-1H-benzimidazole; 2-methyl-5-{4-[4-(2-{[2-(trifluoromethyl)-1H-benzimidazol-4-yl]oxy}ethyl)piperazin-1-yl]-1H-benzimidazol-2-yl}phenol; 2-(trifluoromethyl)-4-{2-[4-(2-{4-[(trifluoromethyl)thio]phenyl}-1H-benzimidazol-4-yl)piperazin-1-yl]ethoxy}-1H-benzimidazole; 2-(4-fluorophenyl)-4-[4-(2-{[2-(trifluoromethyl)-1H-benzimidazol-4-yl]oxy}ethyl)piperazin-1-yl]-1H-benzimidazole; (4-{4-[4-(2-{[2-(trifluoromethyl)-1H-benzimidazol-4-yl]oxy}ethyl)piperazin-1-yl]-1H-benzimidazol-2-yl}phenyl)amine; 2-[4-(trifluoromethoxy)phenyl]-4-[4-(2-{[2-(trifluoromethyl)-1H-benzimidazol-4-yl]oxy}ethyl)piperazin-1-yl]-1H-benzimidazole; 2-(4-cyclohexylphenyl)-4-[4-(2-{[2-(trifluoromethyl)-1H-benzimidazol-4-yl]oxy}ethyl)piperazin-1-yl]-1H-benzimidazole; 2-[4-(methylthio)phenyl]-4-[4-(2-{[2-(trifluoromethyl)-1H-benzimidazol-4-yl]oxy}ethyl)piperazin-1-yl]-1H-benzimidazole; 2-[4-(benzyloxy)phenyl]-4-[4-(2-{[2-(trifluoromethyl)-1H-benzimidazol-4-yl]oxy}ethyl)piperazin-1-yl]-1H-benzimidazole; 2-(4-iodophenyl)-4-[4-(2-{[2-(trifluoromethyl)-1H-benzimidazol-4-yl]oxy}ethyl)piperazin-1-yl]-1H-benzimidazole; 4-{4-[4-(2-{[2-(trifluoromethyl)-1H-benzimidazol-4-yl]oxy}ethyl)piperazin-1-yl]-1H-benzimidazol-2-yl}benzenesulfonamide; 2-(4-propoxyphenyl)-4-[4-(2-{[2-(trifluoromethyl)-1H-benzimidazol-4-yl]oxy}ethyl)piperazin-1-yl]-1H-benzimidazole; 2-[4-(hexyloxy)phenyl]-4-[4-(2-{[2-(trifluoromethyl)-1H-benzimidazol-4-yl]oxy}ethyl)piperazin-1-yl]-1H-benzimidazole; 2-(4-propylphenyl)-4-[4-(2-{[2-(trifluoromethyl)-1H-benzimidazol-4-yl]oxy}ethyl)piperazin-1-yl]-1H-benzimidazole; 2-[4-(methylsulfonyl)phenyl]-4-[4-(2-{[2-(trifluoromethyl)-1H-benzimidazol-4-yl]oxy}ethyl)piperazin-1-yl]-1H-benzimidazole; 2-(4-hexylphenyl)-4-[4-(2-{[2-(trifluoromethyl)-1H-benzimidazol-4-yl]oxy}ethyl)piperazin-1-yl]-1H-benzimidazole; 2-[4-(heptyloxy)phenyl]-4-[4-(2-{[2-(trifluoromethyl)-1H-benzimidazol-4-yl]oxy}ethyl)piperazin-1-yl]-1H-benzimidazole; N-butyl-4-{4-[4-(2-{[2-(trifluoromethyl)-1H-benzimidazol-4-yl]oxy}ethyl)piperazin-1-yl]-1H-benzimidazol-2-yl}aniline; Phenyl-[4-(4-{4-[2-(2-trifluoromethyl-1H-benzoimidazol-4-yloxy)-ethyl]-piperazin-1-yl}-1H-benzoimidazol-2-yl)-phenyl]-methanone; phenyl(4-{4-[4-(2-{[2-(trifluoromethyl)-1H-benzimidazol-4-yl]oxy}ethyl)piperazin-1-yl]-1H-benzimidazol-2-yl}phenyl)methanone; 2-(trifluoromethyl)-4-(2-{4-[2-(4-vinylphenyl)-1H-benzimidazol-4-yl]piperazin-1-yl}ethoxy)-1H-benzimidazole; 2-(4-pentylphenyl)-4-[4-(2-{[2-(trifluoromethyl)-1H-benzimidazol-4-yl]oxy}ethyl)piperazin-1-yl]-1H-benzimidazole; 2-(3-thienyl)-4-[4-(2-{[2-(trifluoromethyl)-1H-benzimidazol-4-yl]oxy}ethyl)piperazin-1-yl]-1H-benzimidazole; 2-(4-butylphenyl)-4-[4-(2-{[2-(trifluoromethyl)-1H-benzimidazol-4-yl]oxy}ethyl)piperazin-1-yl]-1H-benzimidazole; 4-(4-{4-[4-(2-{[2-(trifluoromethyl)-1H-benzimidazol-4-yl]oxy}ethyl)piperazin-1-yl]-1H-benzimidazol-2-yl}phenoxy)phenol; 2-[5-(methylthio)-2-thienyl]-4-[4-(2-{[2-(trifluoromethyl)-1H-benzimidazol-4-yl]oxy}ethyl)piperazin-1-yl]-1H-benzimidazole; 2-(4-phenoxyphenyl)-4-[4-(2-{[2-(trifluoromethyl)-1H-benzimidazol-4-yl]oxy}ethyl)piperazin-1-yl]-1H-benzimidazole; 2-cyclohexyl-4-[4-(2-{[2-(trifluoromethyl)-1H-benzimidazol-4-yl]oxy}ethyl)piperazin-1-yl]-1H-benzimidazole; 2-(5-nitro-2-thienyl)-4-[4-(2-{[2-(trifluoromethyl)-1H-benzimidazol-4-yl]oxy}ethyl)piperazin-1-yl]-1H-benzimidazole; 2-(4-butoxyphenyl)-4-[4-(2-{[2-(trifluoromethyl)-1H-benzimidazol-4-yl]oxy}ethyl)piperazin-1-yl]-1H-benzimidazole; 2-(4-nitrophenyl)-4-[4-(2-{[2-(trifluoromethyl)-1H-benzimidazol-4-yl]oxy}ethyl)piperazin-1-yl]-1H-benzimidazole; 2-(4-tert-butylphenyl)-4-[4-(2-{[2-(trifluoromethyl)-1H-benzimidazol-4-yl]oxy}ethyl)piperazin-1-yl]-1H-benzimidazole; 2-(4-tert-butylphenyl)-4-[4-(2-{[2-(trifluoromethyl)-1H-benzimidazol-4-yl]oxy}ethyl)piperazin-1-yl]-1H-benzimidazole; 2-[5-(4-fluorophenyl)-2-thienyl]-4-[4-(2-{[2-(trifluoromethyl)-1H-benzimidazol-4-yl]oxy}ethyl)piperazin-1-yl]-1H-benzimidazole; 2-[5-(4-methoxyphenyl)-2-thienyl]-4-[4-(2-{[2-(trifluoromethyl)-1H-benzimidazol-4-yl]oxy}ethyl)piperazin-1-yl]-1H-benzimidazole; 2-(4-ethoxyphenyl)-4-[4-(2-{[2-(trifluoromethyl)-1H-benzimidazol-4-yl]oxy}ethyl)piperazin-1-yl]-1H-benzimidazole; 2-(4-methoxyphenyl)-4-[4-(2-{[2-(trifluoromethyl)-1H-benzimidazol-4-yl]oxy}ethyl)piperazin-1-yl]-1H-benzimidazole; 2-[4-(1H-pyrrol-1-yl)phenyl]-4-[4-(2-{[2-(trifluoromethyl)-1H-benzimidazol-4-yl]oxy}ethyl)piperazin-1-yl]-1H-benzimidazole; N,N-diethyl-4-{4-[4-(2-{[2-(trifluoromethyl)-1H-benzimidazol-4-yl]oxy}ethyl)piperazin-1-yl]-1H-benzimidazol-2-yl}aniline; 2-{5-[1-methyl-3-(trifluoromethyl)-1H-pyrazol-5-yl]-2-thienyl}-4-[4-(2-{[2-(trifluoromethyl)-1H-benzimidazol-4-yl]oxy}ethyl)piperazin-1-yl]-1H-benzimidazole; 4-{4-[4-(2-{[2-(trifluoromethyl)-1H-benzimidazol-4-yl]oxy}ethyl)piperazin-1-yl]-1H-benzimidazol-2-yl}benzonitrile; N-methyl-4-{4-[4-(2-{[2-(trifluoromethyl)-1H-benzimidazol-4-yl]oxy}ethyl)piperazin-1-yl]-1H-benzimidazol-2-yl}aniline; 2-(5-methyl-2-thienyl)-4-[4-(2-{[2-(trifluoromethyl)-1H-benzimidazol-4-yl]oxy}ethyl)piperazin-1-yl]-1H-benzimidazole; 2-(4-bromophenyl)-4-[4-(2-{[2-(trifluoromethyl)-1H-benzimidazol-4-yl]oxy}ethyl)piperazin-1-yl]-1H-benzimidazole; 2-biphenyl-4-yl-4-[4-(2-{[2-(trifluoromethyl)-1H-benzimidazol-4-yl]oxy}ethyl)piperazin-1-yl]-1H-benzimidazole; 2-(5-pyridin-2-yl-2-thienyl)-4-[4-(2-{[2-(trifluoromethyl)-1H-benzimidazol-4-yl]oxy}ethyl)piperazin-1-yl]-1H-benzimidazole; 2-[4-(pentyloxy)phenyl]-4-[4-(2-{[2-(trifluoromethyl)-1H-benzimidazol-4-yl]oxy}ethyl)piperazin-1-yl]-1H-benzimidazole; 2-(4-ethylphenyl)-4-[4-(2-{[2-(trifluoromethyl)-1H-benzimidazol-4-yl]oxy}ethyl)piperazin-1-yl]-1H-benzimidazole; 2-(5-bromo-2-thienyl)-4-[4-(2-{[2-(trifluoromethyl)-1H-benzimidazol-4-yl]oxy}ethyl)piperazin-1-yl]-1H-benzimidazole; 2-(4-isopropylphenyl)-4-[4-(2-{[2-(trifluoromethyl)-1H-benzimidazol-4-yl]oxy}ethyl)piperazin-1-yl]-1H-benzimidazole; N-(4-{4-[4-(2-{[2-(trifluoromethyl)-1H-benzimidazol-4-yl]oxy}ethyl)piperazin-1-yl]-1H-benzimidazol-2-yl}phenyl)acetamide; 2-(4-methylphenyl)-4-[4-(2-{[2-(trifluoromethyl)-1H-benzimidazol-4-yl]oxy}ethyl)piperazin-1-yl]-1H-benzimidazole; 2-(5-chloro-2-thienyl)-4-[4-(2-{[2-(trifluoromethyl)-1H-benzimidazol-4-yl]oxy}ethyl)piperazin-1-yl]-1H-benzimidazole; 2-(4-chlorophenyl)-4-[4-(2-{[2-(trifluoromethyl)-1H-benzimidazol-4-yl]oxy}ethyl)piperazin-1-yl]-1H-benzimidazole; 3-[4-(4-{4-[2-(2-Trifluoromethyl-1H-benzoimidazol-4-yloxy)-ethyl]-piperazin-1-yl}-1H-benzoimidazol-2-yl)-phenoxy]-phenol; 3-(4-{4-[4-(2-{[2-(trifluoromethyl)-1H-benzimidazol-4-yl]oxy}ethyl)piperazin-1-yl]-1H-benzimidazol-2-yl}phenoxy)phenol; 2-(2-thienyl)-4-[4-(2-{[2-(trifluoromethyl)-1H-benzimidazol-4-yl]oxy}ethyl)piperazin-1-yl]-1H-benzimidazole; N,N-dimethyl-4-{4-[4-(2-{[2-(trifluoromethyl)-1H-benzimidazol-4-yl]oxy}ethyl)piperazin-1-yl]-1H-benzimidazol-2-yl}aniline; 4-(2-{4-[2-(3-thienyl)-1H-benzimidazol-4-yl]piperazin-1-yl}ethoxy)-1,3-dihydro-2H-benzimidazole-2-thione; 4-(2-{4-[2-(1-naphthyl)-1H-benzimidazol-4-yl]piperazin-1-yl}ethoxy)-1,3-dihydro-2H-benzimidazole-2-thione; 4-(2-{4-[2-(2-naphthyl)-1H-benzimidazol-4-yl]piperazin-1-yl}ethoxy)-1,3-dihydro-2H-benzimidazole-2-thione; 4-(2-{4-[2-(3-aminophenyl)-1H-benzimidazol-4-yl]piperazin-1-yl}ethoxy)-1,3-dihydro-2H-benzimidazole-2-thione; 4-(2-{4-[2-(3-hydroxy-4-methoxyphenyl)-1H-benzimidazol-4-yl]piperazin-1-yl}ethoxy)-1,3-dihydro-2H-benzimidazole-2-thione; 4-(2-{4-[2-(3-hydroxy-4-methylphenyl)-1H-benzimidazol-4-yl]piperazin-1-yl}ethoxy)-1,3-dihydro-2H-benzimidazole-2-thione; 4-[2-(4-{2-[4-(trifluoromethyl)phenyl]-1H-benzimidazol-4-yl}piperazin-1-yl)ethoxy]-1,3-dihydro-2H-benzimidazole-2-thione; 4-[2-(4-{2-[3-(trifluoromethyl)phenyl]-1H-benzimidazol-4-yl}piperazin-1-yl)ethoxy]-1,3-dihydro-2H-benzimidazole-2-thione; 4-[2-(4-{2-[3,5-bis(trifluoromethyl)phenyl]-1H-benzimidazol-4-yl}piperazin-1-yl)ethoxy]-1,3-dihydro-2H-benzimidazole-2-thione; 4-(2-{4-[2-(4-ethylphenyl)-1H-benzimidazol-4-yl]piperazin-1-yl}ethoxy)-1,3-dihydro-2H-benzimidazole-2-thione; 4-(2-{4-[2-(4-phenoxyphenyl)-1H-benzimidazol-4-yl]piperazin-1-yl}ethoxy)-1,3-dihydro-2H-benzimidazole-2-thione; 4-(2-{4-[2-(2,4-dimethylphenyl)-1H-benzimidazol-4-yl]piperazin-1-yl}ethoxy)-1,3-dihydro-2H-benzimidazole-2-thione; 4-(2-{4-[2-(3,4-dimethoxyphenyl)-1H-benzimidazol-4-yl]piperazin-1-yl}ethoxy)-1,3-dihydro-2H-benzimidazole-2-thione; 4-(2-{4-[2-(3,5-dimethylphenyl)-1H-benzimidazol-4-yl]piperazin-1-yl}ethoxy)-1,3-dihydro-2H-benzimidazole-2-thione; 4-(2-{4-[2-(3,5-difluorophenyl)-1H-benzimidazol-4-yl]piperazin-1-yl}ethoxy)-1,3-dihydro-2H-benzimidazole-2-thione; 4-(2-{4-[2-(3-methylphenyl)-1H-benzimidazol-4-yl]piperazin-1-yl}ethoxy)-1,3-dihydro-2H-benzimidazole-2-thione; 4-(2-{4-[2-(3-bromophenyl)-1H-benzimidazol-4-yl]piperazin-1-yl}ethoxy)-1,3-dihydro-2H-benzimidazole-2-thione; 4-(2-{4-[2-(2,4-dichlorophenyl)-1H-benzimidazol-4-yl]piperazin-1-yl}ethoxy)-1,3-dihydro-2H-benzimidazole-2-thione; 4-(2-{4-[2-(4-bromophenyl)-1H-benzimidazol-4-yl]piperazin-1-yl}ethoxy)-1,3-dihydro-2H-benzimidazole-2-thione; 4-(2-{4-[2-(2,3,6-trifluorophenyl)-1H-benzimidazol-4-yl]piperazin-1-yl}ethoxy)-1,3-dihydro-2H-benzimidazole-2-thione; 4-(2-{4-[2-(diphenylmethyl)-1H-benzimidazol-4-yl]piperazin-1-yl}ethoxy)-1,3-dihydro-2H-benzimidazole-2-thione; 4-(2-{4-[2-(2,2-diphenylethyl)-1H-benzimidazol-4-yl]piperazin-1-yl}ethoxy)-1,3-dihydro-2H-benzimidazole-2-thione; 4-(2-{4-[2-(2,4-dimethoxyphenyl)-1H-benzimidazol-4-yl]piperazin-1-yl}ethoxy)-1,3-dihydro-2H-benzimidazole-2-thione; 4-(2-{4-[2-(2,4,6-trimethoxyphenyl)-1H-benzimidazol-4-yl]piperazin-1-yl}ethoxy)-1,3-dihydro-2H-benzimidazole-2-thione; 4-(2-{4-[2-(4-methoxyphenyl)-1H-benzimidazol-4-yl]piperazin-1-yl}ethoxy)-1,3-dihydro-2H-benzimidazole-2-thione; 4-{2-[4-(2-pyridin-4-yl-1H-benzimidazol-4-yl)piperazin-1-yl]ethoxy}-1,3-dihydro-2H-benzimidazole-2-thione; 4-{2-[4-(2-pyridin-3-yl-1H-benzimidazol-4-yl)piperazin-1-yl]ethoxy}-1,3-dihydro-2H-benzimidazole-2-thione; 3-[4-(4-{2-[(2-thioxo-2,3-dihydro-1H-benzimidazol-4-yl)oxy]ethyl}piperazin-1-yl)-1H-benzimidazol-2-yl]benzonitrile; 4-[4-(4-{2-[(2-thioxo-2,3-dihydro-1H-benzimidazol-4-yl)oxy]ethyl}piperazin-1-yl)-1H-benzimidazol-2-yl]benzonitrile; 4-{2-[4-(2-pyridin-2-yl-1H-benzimidazol-4-yl)piperazin-1-yl]ethoxy}-1,3-dihydro-2H-benzimidazole-2-thione; or a stereoisomer or pharmaceutically acceptable salt thereof.

15. A pharmaceutical composition comprising the compound of claim 1 or a pharmaceutically acceptable salt thereof, and a pharmaceutically acceptable carrier.

16. A pharmaceutical composition comprising the compound of claim 14 or a pharmaceutically acceptable salt thereof, and a pharmaceutically acceptable carrier.

17. A pharmaceutical composition, comprising: a) a compound of claim 1 or a pharmaceutically acceptable salt thereof; and b) an additional active agent selected from the group consisting of at least one of androgen, estrogen, progesterone, antiestrogen, antiprogestogen, testosterone, angiotensin-converting enzyme inhibitor, angiotensin II-receptor antagonist, renin inhibitor, bisphosphonate, growth hormone secretagogue, 5a-reductase 2 inhibitor, a 5a-reductase 1 inhibitor, dual inhibitors of 5a-reductase 1 and 5a-reductase 2, antiandrogen, alpha-1 blockers, growth hormone, and luteinizing hormone releasing compound.

18. A pharmaceutical composition, comprising: a) a compound of claim 14 or a pharmaceutically acceptable salt thereof; and b) an additional active agent selected from the group consisting of at least one of androgen, estrogen, progesterone, antiestrogen, antiprogestogen, testosterone, angiotensin-converting enzyme inhibitor, angiotensin II-receptor antagonist, renin inhibitor, bisphosphonate, growth hormone secretagogue, 5a-reductase 2 inhibitor, a 5a-reductase 1 inhibitor, dual inhibitors of 5a-reductase 1 and 5a-reductase 2, antiandrogen, alpha-1 blockers, growth hormone, and luteinizing hormone releasing compound.

19. A method for modulating the activity of a Gonadotropin Releasing Hormone receptor, comprising contacting said receptor with an effective amount of a compound according to claim 1.

20. The method of claim 19, further comprising determining the activity of said receptor.

21. The method of claim 20, wherein said determination is made before said contacting step.

22. The method of claim 20, wherein said determination is made after said contacting step.

23. A method for treating a patient suspected of suffering from a condition associated with excessive Gonadotropin Releasing Hormone receptor activity, comprising the step of administering to the patient a therapeutically effective amount of a compound according to claim 1.

24. The method of claim 23, wherein said condition is prostate cancer, endometriosis, uterine fibroids, uterine cancer, breast cancer, ovarian cancer, testicular cancer, primary hirsutism, or LH surge.
